SEQUENCE LISTING (2 sequences)

SEQ ID NO: 1 - DNA, 21 bp, Artificial Sequence
  Synthetic
agaccaggataagaaacgcag

SEQ ID NO: 2 - DNA, 20 bp, Artificial Sequence
  Synthetic
tttccgcctcaatgatagct

(19) United States
(12) Patent Application Publication
(10) Pub. No.: US 2013/0324543 A1
(43) Pub. Date: Dec. 5, 2013

(54) REVERSAL OF HIGH BREAST CANCER RISK IN MAMMALS EXPOSED TO ESTROGENIC CHEMICALS IN UTERO BY ADULT EXPOSURE TO HDAC AND DNMT INHIBITORS

(71) Applicant: Georgetown University, Washington, DC (US)

(72) Inventors: Leena Hilakivi-Clarke, Rockville, MD (US); Anni Warri, Bethesda, MD (US); Dominic Kim, West Friendship, MD (US)

(73) Assignee: Georgetown University, Washington, DC (US)

(21) Appl. No.: 13/857,583

(22) Filed: Apr. 5, 2013

Related U.S. Application Data
(60) Provisional application No. 61/621,308, filed on Apr. 6, 2012.

(51) Int. Cl. C12Q 1/68

(52) U.S. Cl. CPC C12Q 1/6886
USPC 514/248; 435/6.11

(57) ABSTRACT

Methods of reducing breast cancer risk in an individual exposed in utero to an elevated estrogenic environment, where the individuals are identified by obtaining a measurement of at least one of: (a) DNMT1 expression; (b) methylation of normally unmethylated CpG islands of tumor suppressor genes; and (c) polycomb target genes, in cells obtained from the individual and comparing the measurement to a statistical level of a population not exposed in utero to an elevated estrogenic environment. Methods of reducing breast cancer risk in such an individual can be reduced by administering at least one DNA methyltransferase (DNMT) and/or histone deacetylase (HDAC) inhibitor. The administration of these inhibitors may be part of a treatment regime which may include the administration of at least one anti-cancer agent.

This application claims the benefit of U.S. Provisional Application No. 61/621,308, filed on Apr. 6, 2012, the contents of which are incorporated by reference herein, in their entireties and for all purposes.

### REFERENCE TO U.S. GOVERNMENT SUPPORT

This work is supported by a grant from the Federal Drug Administration (Grant No. U01FD003873) and a grant from the National Cancer Institute (Grant No. U54 CA100970). The United States has certain rights in the invention.

### FIELD OF THE INVENTION

The subject matter described therein relates to the fields of molecular biology and medicine. More specifically, it relates to the field of cancer research, especially breast cancer diagnosis and treatment.

### BACKGROUND OF THE INVENTION

Breast cancer is the most common cancer in women, affecting 1 out of every 8 women in the US during their life time. Exposure to elevated estrogenic environment during pregnancy increases breast cancer risk among female offspring in animal models and, for many reasons, is believed to do so in humans. These exposures leave permanent marks in estrogen-responsive cells, which can be used to indicate that these cells were exposed to an elevated estrogenic environment in utero; such permanent marks are passed on into daughter cells upon cell division during mammary gland growth. These marks bear biological and functional consequences, including that the cells in the mammary gland will respond differently to ovarian estrogens, when their production starts at puberty onset, and factors which initiate malignant transformation of cells (i.e. cancer), such as carcinogens, radiation and inflammation. The etiology of breast cancer remains largely unconfirmed. For example, this disease can only partially be explained by either inherited mutations or reproductive risk factors, as most women diagnosed with breast cancer don't have any of these risk factors. However, hormones are clearly implicated in the etiology of breast cancer. While attempts to link women's exposure to endocrine disruptors (EDCs) to breast cancer have led to conflicting results, the possibility remains that more harmful than adult exposure to EDCs may be exposure during fetal period, as fetal tissues are more vulnerable than adult ones. Thus, there is a need to identify women who have been exposed to EDC in utero, because they are shown to be at increased risk of developing breast cancer. There is also a need to determine if healthy, but high risk women can have their risk of developing breast cancer reduced by an agent that can be safely administered to healthy women before they develop the cancer.

### SUMMARY OF THE INVENTION

In one aspect, an exemplary embodiment provides a method of reducing breast cancer risk in an individual exposed in utero to an elevated estrogenic environment, wherein the individual is identified by having at least one of the following parameters: (a) elevated DNMT1 expression, or (b) methylation of normally unmethylated CpG islands of tumor suppressor genesor or (c) polycomb target genes, or (d) down-regulation of miRNAs in cells obtained from the blood or other peripheral tissue or breast (nipple aspirate fluid, ductal lavage or biopsy). As the only reported in utero EDC exposure in women known to lead to increased breast cancer risk is being a daughter of a mother who took the synthetic estrogen diethylstilbestrol (DES) during pregnancy (15, 125), differences in these women in (a) to (d), compared to non-exposed daughters, may be useful in identifying other women who were exposed to some other EDCs during fetal period. In addition, preclinical studies will be highly useful in identifying individuals exposed to elevated in utero estrogenic environment. In exemplary embodiments, the statistical level that can be used can be a certain percentage of the mean value, such as about 150% or about 200%, or it can be based on a confidence interval around the mean of the population, such as about 75%, about 90% or about 95%.

In another aspect, an exemplary embodiment provides a method of reducing breast cancer risk in an individual exposed in utero to an elevated estrogenic environment by administering at least one DNA methyltransferase (DNMT) and/or histone deacetylase (HDAC) inhibitor to an individual identified by obtaining a measurement of at least one of: (a) DNMT1 expression; (b) methylation of normally unmethylated CpG islands of tumor suppressor genes or (c) polycomb target genes, or down-regulation of miRNAs in cells obtained from the blood or breast (nipple aspirate fluid, ductal lavage or biopsy).

In another aspect, an exemplary embodiment provides a method of reducing breast cancer risk in an individual exposed in utero to an elevated estrogenic environment by administering at least one of a DNA methyltransferase (DNMT) and/a histone deacetylase (HDAC) inhibitor to an individual identified by obtaining a measurement of at least one of: (a) DNMT1 expression; (b) methylation of normally unmethylated CpG islands of tumor suppressor genes; or (c) polycomb target genes, or (d) down-regulation of miRNAs in cells obtained from the blood or breast (nipple aspirate fluid, ductal lavage or biopsy.

In another aspect, an exemplary embodiment provides a method of reducing breast cancer risk in an individual exposed in utero to an elevated estrogenic environment by administering at least one of a DNA methyltransferase (DNMT) and a histone deacetylase (HDAC) inhibitor to an individual as part of a treatment regimen which may include the administration of at least one cancer treatment agent.

The applicability of the present teachings to other areas will become apparent from the detailed description provided hereinafter. It should be understood that the detailed description and specific examples, while indicating certain embodiments of the present teachings, are intended for purposes of illustration only and are not intended to limit the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows that prepubertal exposure to DMT inhibitor 5-aza reverses changes in gene expression in adult mammary glands of in utero E2 exposed rats.

FIG. 2 shows maternal exposure to 0.1 ppm EE2 during pregnancy increases DNA methylransferase expression in female offspring (F1 generation), granddaughters (F2) and great granddaughters (F3).

FIG. 3 shows maternal exposure to GEN reduces the latency of mammary tumor development. Adult exposure to HDAC+DNMT inhibitors reverses this effect.

FIG. 4 shows maternal exposure to EE2 or GEN during pregnancy increases mammary tumorigenesis among female offspring. Adult exposure to HDAC and DNMT inhibitors prevents this increase.

FIG. 5 shows adult exposure to HDAC+DNMT inhibitors reverses the increase in mammary tumor burden in female offspring of dams fed EE2 or GEN containing diet during pregnancy.

FIG. 6 shows maternal exposure to 0.1 ppm EE2 or 500 ppm genistein (GEN) during pregnancy increases DNMT1 expression in the mammary glands of female offspring, but no changes are seen in the tumors. Adult exposure to HDAC+DNMT inhibitors reverses the increase in the mammary glands of the EE2 group. In the tumors, HDAC+DNMT1 inhibitor increases DNMT1 expression, but only in the control mice.

FIG. 7 shows mammary tumorigenesis in F1 generation exposed to E2 in utero. Mammary tumor incidence is higher in the rats exposed to E2 in utero (p<0.023). Tumor multiplicity (p<0.022) and total tumor burden (p<0.001) are also higher in in utero E2 exposed rats than in the controls.

FIG. 8 shows mammary glands of 21-day-old rats exposed to 10 μg E2 in utero contain a larger parenchyma and more TEBs, exhibit more advanced ductal elongation, and are larger than the glands of the control rats.

FIG. 9 shows preliminary DDN analysis of control vs. in utero E2 exposed adult mammary glands.

FIG. 10 shows differences in gene expression in mammary glands of adult rats exposed to a control or birth weight increasing high fat diet in utero, and demethylating agent 5-aza or vehicle before puberty. 5-aza reversed down-regulation of PR, Rassf1 and RAR beta in high fat, high birthweight group.

FIG. 11 shows TEB number on postnatal day 21 in F1 and F2 generations. Matemal exposures during pregnancy: CON=control, E2=estradiol-supplemented, HF=high-fat diet n=3-6 offspring/group, mean±SEM. Statistical analysis was done using ANOVA followed by between group comparisons using Tukey test.

FIG. 12 shows the outline of the study to assess the effects of in utero exposure of mice to EE2, BPA and GEN.

FIG. 13 shows the body weights of in utero EDC exposed female and male mice at puberty.

FIG. 14 shows puberty onset of female mice exposed in utero to ECD.

FIG. 15 shows In utero EDC exposures did not affect mammary gland development in male mice. This embryonal development is very sensitive to uterine environment. Mammary glands of male C57BI/6 mice have rudimentary ductal epithelium at birth. No abnormal growth was observed at the time of puberty onset as a result of the in utero ECD exposures.

FIG. 15 shows male mice (of some mouse strains) typically have rudimentary mammary glands at the time of their birth, and lack nipples.

FIG. 16 shows mammary gland epithelial area and TEB number on PND 27.

FIG. 17 shows total mammary gland epithelial area and total number of TEBs on PND 27.

FIG. 18 shows cumulative tumor growth in mice exposed in utero to EE2, GEN and BPA.

FIG. 19 shows effect of HDAC+DNMT inhibitors on mammary tumorigenesis.

FIG. 20 shows mammary tumors in different generation mice with high fat diets.

FIG. 21 shows mammary tumors in different generation mice exposed to EE2.

FIG. 22 shows DNMT1 levels in various generations.

FIG. 23 shows study design. (a) Pregnant Sprague-Dawley rat dams (F0, n=10-12) were fed one of the following experimental diets: High Fat (HF), EE2-supplemented (EE2) or control. The HF group was fed this experimental diet throughout pregnancy while the EE2 group was fed this experimental diet from pregnancy day 14 to 20. F1 and F2 HF or EE2 exposed females were mated on PND 60 with males from the same group to produce the F2 and F3 generations. All F1 and F2 pregnant dams were fed control diet for the extent of pregnancy. (b) Outcross experiments were performed by mating F1 females or males exposed to EE2 or HF to control males or females. All F1 pregnant dams were fed control diet for the extent of pregnancy. (c) Mammary gland tissues were collected on PND21 (n=6) and PND50 (n=6) for morphological analysis and mRNA extraction. Mammary tumors were induced on PND50 in F1, F2 and F3 generation females (n=12-27/group) by administration by oral gavage of 10 mg of 9,12-dimethylbenz[a]anthracene (DMBA). Rats were examined for mammary tumors by palpation once per week, starting on 3 weeks post-DMBA and continued for 20 weeks post-DMBA.

FIG. 24 shows HF offspring body weight and litter size in F1-F3 generations. (a) Body weight (g) of pups on PND2 offspring (n=4-15). (b) Number of pups per litter (n=5-12). All data are mean±s.e.m.: t-test or Mann-Whitney Rank Sum test (a, b).

FIG. 25 shows EE2 offspring body weight and litter size in F1-F3 generations. (a) Body weight (g) of pups on PND2 offspring (n=3-15). (b) Number of pups per litter (n=5-12). All data are meant s.e.m.: t-test or Mann-Whitney Rank Sum test (a, b).

FIG. 26 shows maternal exposure to 0.1 ppm EE2 during pregnancy increases mammary cancer risk in F1-F3 generation offspring.

FIG. 27 shows response to TAM among control and in utero EE2 exposed rats during 14-week follow-up. 50% of offspring exposed to 0.1 ppm EE2 in utero exhibit acquired resistance to TAM, compared to 7% in the control group.

FIG. 28 shows suppression of miRNA expression in the mammary gland of 50-day-old offspring of control (panel of first 4) and EE2 exposed dams (panel of last 4). All these miRNAs also are suppressed in MCF-7 breast cancer cells treated with EE2.

FIG. 29 shows inter-individual variability in E2 levels among 286 healthy pregnant women on gestation week 12 and 32.

FIG. 30 shows maternal exposure to HF or EE2 containing diet during pregnancy increases DNMT1 expression in the mammary glands of F1-F3 offspring.

FIG. 31 shows changes in methylation in F1-F3 generation of rat dams exposed to EE2 containing diet during pregnancy. Brown bars indicate methylation that is unique for EE2 offspring, red bars are unique for controls, and blue bars are common to both.

FIG. 32 shows adult exposure to HDAC+DNMT inhibitors prevents the increase in mammary tumorigenesis in mice exposed to estrogenic compounds (EE2 or genistein) in utero.

FIG. 33 shows study design for determining the effects of in utero exposure to EE2 at 0.1 ppm in the diet.

FIG. 34 shows increased risk of mammary cancer in rats exposed in utero to 0.1 ppm of EE2 as the percentage of rats with mammary tumors.

FIG. 35 shows increased risk of mammary cancer in mice exposed in utero to 0.1 ppm of EE2 as the percentage of mice with mammary tumors.

FIG. 36 shows increased risk of mammary cancer in rats exposed in utero to 0.1 ppm of EE2 as the cumulative tumor burden.

FIG. 37 shows maternal exposure to EE2 during pregnancy increases the expression of DNMT1 in the mammary glands of adult offspring.

FIG. 38 shows methylation levels in 11 CpG islands in ER-a promoter region.

FIG. 39 shows maternal exposure to E2 during pregnancy increases ER-a protein levels.

FIG. 40 shows maternal exposure to EE2 during pregnancy down-regulates miRNAs, which target ER-a.

FIG. 41 shows heatmap of genes altered in MCF-7 human breast cancer cells treated with EE2 and heatmap of the same genes in offsping's mammal glands.

FIG. 42 shows the relationship between miRNAs and genes in mammary glands from adult offspring's where maternal exposure to EE2 during pregnancy down-regulates miRNAs in the off-spring's mammary gland which target ER-a regulated genes that are up-regulated in the EE2 exposed adult offspring's mammary gland.

FIG. 43 shows the relationship between miRNAs and genes in mammary glands from adult offspring's where maternal exposure to EE2 during pregnancy down-regulates miRNAs in the off-spring's mammary gland which target oncogenes that are up-regulated in the EE2 exposed adult offspring's mammary gland.

FIG. 44 shows study design for the evaluation of in utero endocrine disruptor chemical (EDC) exposure's effects on growth and sexual maturation of pups.

FIG. 45 shows puberty onset of female mice exposed in utero to EDC.

FIG. 46 shows mammary gland epithelial area on PND 27.

FIG. 47 shows latency of tumor development Increased mammary tumorigenesis in mice exposed to EE2 or GEN in utero. Adult exposure to HDAC and DNMT Inhibitors reverses the increase.

FIG. 48 shows latency of tumor development in mice treated with HDAC+DNMT inhibitors. Maternal exposure to GEN reduces the latency of mammary tumor development Adult exposure to HDAC+DNMT inhibitors reverses this effect.

FIG. 49 shows mammary tumor incidence in mice exposed to EE2 or GEN in utero.

FIG. 50 shows mammary tumor incidence in mice exposed to EE2 or GEN in utero and HDAC+DNMT inhibitors in adult life. Matemal exposure to EE2 or GEN during pregnancy increases mammary tumorigenesis among female offspring. Adult exposure to HDAC and DNMT inhibitors prevents this increase.

FIG. 51 shows effect of HDAC+DNMT inhibitors on mammary tumorigenesis in control mice.

FIG. 52 shows the effect of HDAC+DNMT inhibitors on mammary tumorigenesis in in utero EE2 exposed mice.

FIG. 53 shows the effect of HDAC+DNMT inhibitors on mammary tumorigenesis in in utero GEN exposed mice. Adult exposure to HDAC+DNMT inhibitors reverses the increase in mammary tumor burden in female offspring of dams fed EE2 or GEN containing diet during pregnancy. Permanent changes in expression of DNMT1 in mammary glands & tumors exposed to EDC during embryogenesis.

FIG. 54 shows the expression of DNMT1 mRNA in mammary glands. F for group (2, 45)=7.39, p<0.002; F for interaction (2, 45)=5.49, p<0.007.

FIG. 55 shows the expression of DNMT1 mRNA in mammary tumors in mice exposed to EE2 or GEN in utero. F for group (2, 34)=7.68, p<0.002. F for interaction (2, 34)=3.57, p<0.039

FIG. 56 shows the experimental design.

FIG. 57 shows MDA adduct levels in normal mammary glands in the F1-F3 generation offspring.

FIG. 58 shows the expression of cyp1b1 qPCR in the F3 generation.

FIG. 59 shows cell proliferation in the F1, F2 and F3 generations.

### DETAILED DESCRIPTION OF THE INVENTION

An exposure to an elevated estrogenic environment during pregnancy increases breast cancer risk among female offspring in animal models (1-11) and, for various reasons is believed to do so also in humans (12-15). Estrogenic exposures which are shown to increase offspring's breast cancer risk in animal studies include natural estradiol produced by ovaries and during pregnancy, the placenta, synthetic estrogen diethylstilbestrol (DES), which was used by millions of pregnant women between 1940s and 1970s to prevent miscarriage, or endocrine disrupting chemicals (EDCs) present in plants (for example genistein in soy) or in wide variety of manufactured products, such as Bisphenol A (BPA) in plastic (16). Findings regarding DES have been confirmed also in humans (15). The mechanisms involved in mediating the effects of in utero estrogenic environment on later breast cancer risk are not known. These exposures leave a permanent mark in estrogen-sensitive cells that indicates that these cells were once exposed to excess estrogens (17). Consequently, the cells in the mammary gland will respond differently to puberty onset (18) and factors which initiate cancer (e.g., carcinogens).

More than 80,000 chemicals are registered for use in commerce in the United States and an estimated 2,000 new chemicals are introduced annually. These chemicals are used or present as contaminants in everyday items such as foods, personal care products, prescription drugs, household cleaners, and lawn care products. [National Toxicology Program, 2002] Scientists are continually learning more about how these compounds interact with the body and the long term impact of these interactions on our health. Unfortunately, many of these chemicals have been identified as known or suspected endocrine disruptors (EDCs), including diethylstilbestrol (DES), Bisphenol A (BPA) and genistein (GEN). The long term impact of these chemicals on human health is still largely unknown, particularly when the exposure levels are relatively low and do not cause any apparent toxic effects. EDCs may have hormone-like, i.e. estrogenic, antiestrogenic, androgenic, and/or anti-androgenic actions and/or they may disrupt adrenal and/or thyroid functions, too. Other targets might include aryl hydrocarbone receptor (AhR) and estrogen related receptors gamma (ERRγ) (34). The public, scientific and regulatory concern about the potential adverse human health impacts of exposure to EDCs was first identified in several reports published in the early 1990s.

Epigenetic changes form the memory mark and mediate the effects of in utero estrogenic exposures on later cancer risk, perhaps including breast cancer. All cells in different tissues and organs in humans and other multi-cellular organisms originate from a single fertilized cell and thus these cells have identical DNA sequences, although they are morphologically very different and have different functions. Cell differentiation to multiple lineages is governed by both genetic and epigenetic changes, and the latter is influenced by the environment in utero. These early epigenetic processes include DNA methylation and histone modifications. In simple terms, the level of DNA methylation is determined by the presence of methyl groups in CpG islands located mostly in gene's promoter region. The more methylated the islands are, the less the gene can be expressed. These methylation patterns are then inherited to daughter cells by mechanisms which involve DNA methyltransferases (DNMTs). DNA methylation patterns in different cells during fetal development can be modified by maternal exposure to EDCs (18, 19), but there exists a need for studies that have investigated the effect of in utero EDC exposure on DNA methylation in the breast.

Epigenetic changes are thought to be reversible. These changes are common in different cancers, and consequently some cancers are treated with DNMT inhibitors, often in combination with histone deacetylase (HDAC) inhibitors. However, it is not clear what causes DNA methylation of for example tumor suppressor genes (TSGs) or polycomb target genes (PcTGs), but their DNA methylation in peripheral blood or breast fluid cells before any cancers are detected, is strongly predictive of increased breast cancer risk (20-22). In our unpublished studies, we have found, and described here, that in utero estrogenic exposures cause down-regulation and methylation of both TSGs and PcTGs.

Table 1 shows PcGTs which are differentially expressed in the microarray analysis in E2 exposed rats. Expression level also indicates whether these genes are hypo- or hypermethylated. We then determined changes in gene methylation in the mammary glands of 2-month-old rats exposed to excess E2 in utero by using MBDCap-sequencing, which is a methyl-CpG binding domain-based capture method coupled with massive parallel sequencing. Table 1a shows PcTGs and TSGs which are methylated in the mammary glands on postnatal day 50 in rats exposed in utero to EE2 and which remained methylated in F2 and F3 generations.

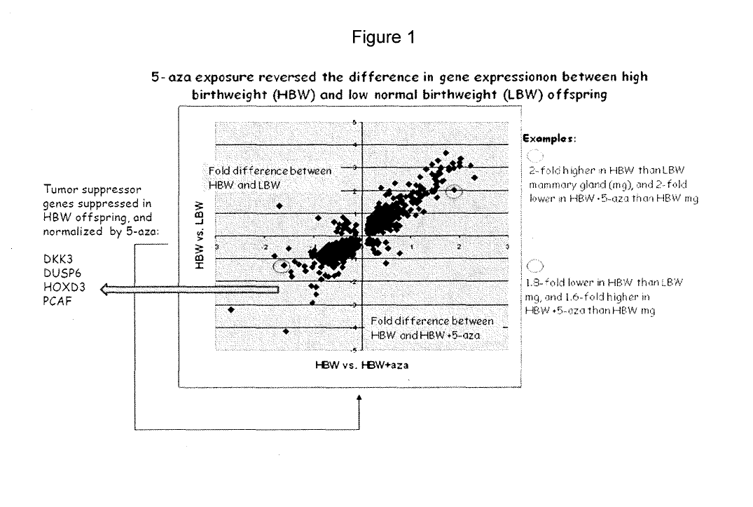

Finally, several genes were down-regulated, including TSGs (FIG. 1), some by methylation, in the adult mammary gland of rats whose mothers were fed high birth weight (HBW) inducing high fat diet during pregnancy, Reduced expression of these genes was reversed if the rats were exposed to DNMT inhibitor 5-aza before puberty onset. Importantly, previous studies show that in utero exposures to endocrine disruptors alter the expression of DNMTs (126, 128) in adult target tissues, but it has not been studied whether these changes include the mammary gland.

DNMTs are enzymes that methylate CpG Islands by adding a methyl group (CH₃) onto the 5-carbon of the cytosine ring within CpG dinucleotides. These enzymes include DNMT1, DNMT3a, and DNMT3b; it is now clear that all three enzymes can act both as maintenance and de novo methylation enzymes (1, 2). Overexpression of DNMT1 induces genomic hypermethylation and loss of imprinting (3), and therefore high DNMT1 protein levels may be responsible for aberrant DNA methylation in cancer (4). RNAi-induced depletion of DNMT1 leads to demethylation of CpG islands in human breast cancer cells (T47D, MDA-MB-231 and Hs578t) (see (5)). DNMT1 and DNMT3b are essential for preventing stem/progenitor cell differentiation, and they both up-regulate polycombs (127). DNMT3b may be important in breast cancer, since it is the most highly overexpressed of the three DNMTs in tumor cells, when compared to normal mammary cells (6). Overexpression of DNMT3b is also associated with basal-like (ER-/PR-/HER2-) breast cancers, and sensitivity of these tumors to cytotoxic chemotherapy can be increased by treating cells with DNMT inhibitors (129). DNMT3a and its cofactor DNMT3L are essential in establishing differential DNA methylation of the paternal and maternal alleles of imprinted genes in mammalian germ cells.

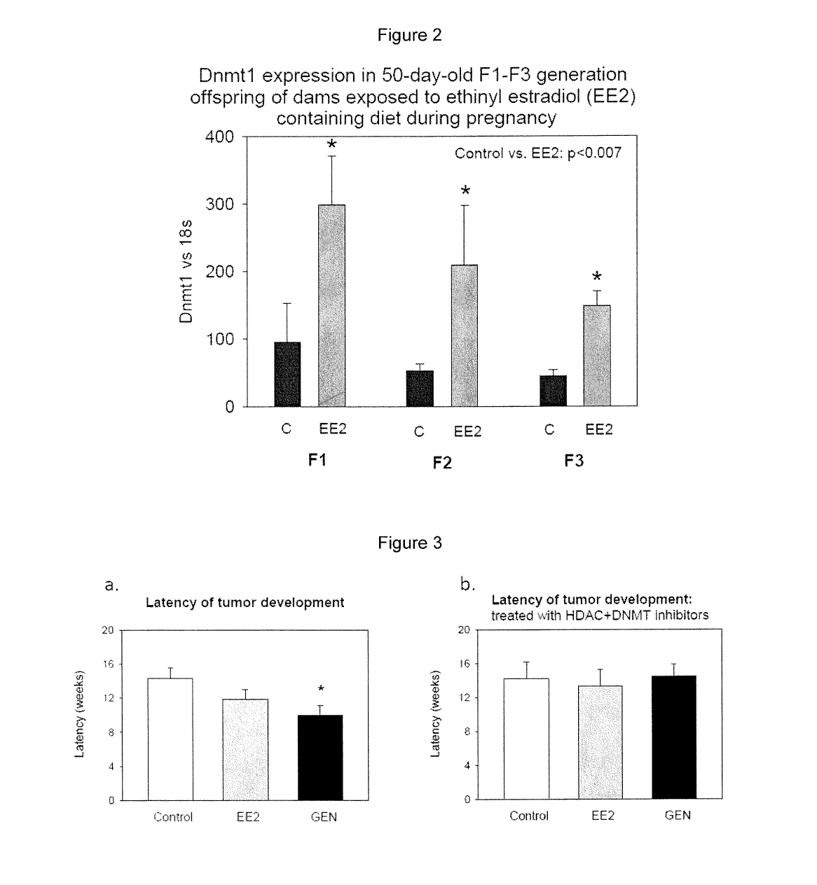

We found that the expression of DNMT1 is elevated in the mammary glands of animals exposed in utero to synthetic estrogen EE2 through their pregnant dam, and the increase persists at least three subsequent generations (FIG. 2). Neither DNMT3a or 3b were altered in the mammary glands of in utero EE2 exposed rats (not shown), although they may be up- or down-regulated in other tissues following in utero estrogenic exposures (126, 128)

TABLE 1
Differentially expressed PcGTs in the mammary glands
of in utero E2 exposed rats on PND 50 (of 30 genes)

| | Fold down-regulated | Fold up-regulated |
|---|---|---|
| | in E2 group | in E2 group |
| Actn1, alpha 1 actin | −5.63 (p < 0.03) | |
| Ccl2, chemokine (C-C motif) | −1.81 (0.00) | |
| ligand 2 | | |
| Ckm, muscle creatine kinase | −7.68 (p < 0.01) | |
| H19/Pro2605, H19fetal liver | −3.28 (p < 0.03) | |
| mRNA (imprinted gene) | | |
| Myh13, heavy polypeptide 13 | −6.08 (p < 0.01) | |
| myosin | | |
| Myl2, light polypeptide 2 | −10.19 (p < 0.02) | |
| myosin | | |
| Pvnlb, parvalbumin | −8.64 (p < 0.03) | |
| Hmgcs2, 3-hydroxy-3- | | 2.46 (p < 0.00) |
| methylglutaryl- | | |
| Coenzyme A synthase 2 | | |
| Lep, Leptin | | 1.59 (p < 0.02) |
| Pdk4, puruvate dehydrogenase | | 1.82 (p < 0.02) |
| kinase isoenzyme 4 | | |

TABLE 1a

| | Abbre- | | Chro- | Fold- | |
|---|---|---|---|---|---|
| Gene | viation | Description | mosome | difference | p-value |
| ADF-ribosylation factor guanine | Arfgef2 | Cell proliferation and inhibition of apoptosis. Neural progenitor | “3” | 0.495238095 | 0.013335783 |
| nucleotide-exchange factor 2 | | cell proliferation | | | |
| BTB [symbol] domain containing 10 | Btbd10 | Inhibition of cell growth, death and adhesion. Down-regulated | “1” | 2.133333333 | [symbol] |
| | | in [symbol] | | | |
| Chemokine (C-C motif) receptor 5 | Ccr5 | PcTG. Cell death, differentiation, growth, migration, and | “3” | 2.35 | [symbol] |
| | | inhibition of apoptosis, proliferation, vascularization, immune | | | |
| | | cell receptor, HIV co-receptor | | | |
| Glutamate receptor, [symbol] , | Grik4 | Excitatory neurotransmission. Involved in Neural progenitor | “3” | [symbol] | 0.005054433 |
| kainate 4 | | cell proliferation, bipolar [symbol] and mental retardation | | | |
| Ofactory receptor 1593 | O [symbol] | Neuronal perception of smell | “13” | [symbol] | [symbol] |
| Ofactory receptor 159 | O [symbol] | Neuronal perception of smell | “1” | [symbol] | [symbol] |
| Ofactory receptor 70 | O [symbol] | Neuronal perception of smell | “1” | [symbol] | [symbol] |
| p31 protein [symbol] -activated | Pak3 | PcTG. Apoptosis, Cell death, differentiation, proliferation | “X” | 0.5 | 0.005143185 |
| kinase 3 | | Over-expressed in several cancers. Involved in Alzheimer | | | |
| | | disease, mental retardation | | | |
| proline rich [symbol] G-carboxyglutamic | P [symbol] 3 | Predictor of metastatic outcome of early stage aggressive | “X” | [symbol] | [symbol] |
| acid 3 | | breast cancer. Involved in mental retardation | | | |
| ribosomal protein [symbol] | [symbol] | Inhibition of apoptosis. Regulation of [symbol] expression by | “3” | [symbol] | [symbol] |
| | | interacting with [symbol] in response to growth factor and | | | |
| | | nutritional signals | | | |
| [symbol] calcium binding protein A11 | [symbol] Ca11 | Cell adhesion apoptosis, differentiation, growth, | “2” | 2.05 | 0.009534013 |
| | | proliferation, inhibition of cell cycle regulation and | | | |
| | | DNA replication. Dual cell growth [symbol] ST00A11 acts | | | |
| | | as either a tumor suppressor or promoter in different | | | |
| | | types of tumors and would play respective roles in | | | |
| | | influencing the proliferation of the cancer cells | | | |
| semaphorin [symbol] A | [symbol] | PcTG. Cell migration and inhibits cell death. Down- | “13” | 2 | [symbol] |
| | | regulation related to cancer drug treatment resistance | | | |
| splicing factor 3a. subunit 3, [symbol] | Sf3a3 | Pre-mRNA splicing | “5” | 0.492753633 | [symbol] |
| solute carrier family [symbol] member [symbol] | [symbol] | Regulation of cell cycle and growth and inhibition of | “3” | 2.052531579 | [symbol] |
| | | cell proliferation. Involved in mood and bipolar disorder, | | | |
| | | schizophrenia, neuropathy | | | |
| translocase of inner | Timm [symbol] a1 | Inhibition of apoptosis. Deafness/dystonia syndrome | “X” | [symbol] | [symbol] |
| mitochondrial membrane | | | | | |
| [symbol] homolog a1 | | | | | |
| [symbol] tRNA methyltransferase [symbol] | [symbol] | tRNA methyl-transferase. Catalyze methyl transfer from | “5” | [symbol] | 0.041752971 |
| homolog | | [symbol] to [symbol] of [symbol] | | | |
| [symbol] -box domain containing | Z [symbol] | Ion binding involved [symbol] attention deficit hyperactivity | “2” | 2.333333333 | 0.03197405 |
| | | disorder | | | |

[symbol]

indicates data missing or illegible when filed

Several DNMT inhibitors can be used in exemplary embodiments, alone or in combination. Exemplary inhibitors include 5-azacytidine (5-azacitidine), 5-aza-2′-deoxycytidine (decitabine), fazarabine, DHAC, Ara-C, zebularine, (−)-epigallocatechin-3-gallate, MG98, RG108, procainamid combinations therefor and the like. The structures of some of these compounds are shown below.

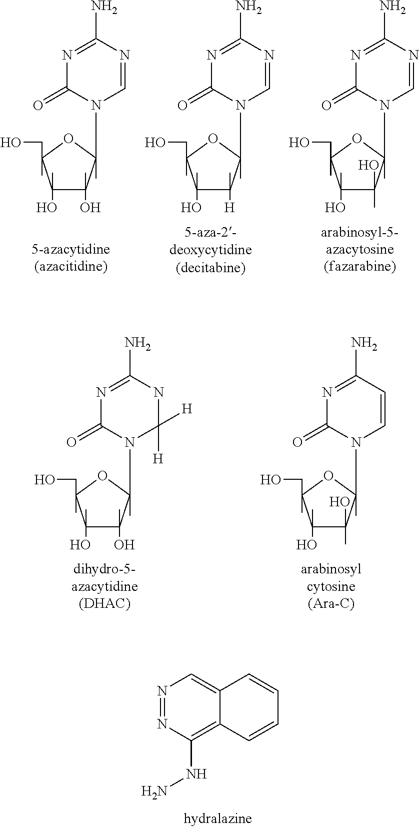

Several Histone deacetylase (HDAC) inhibitors can be used in exemplary embodiments, alone or in combination. Exemplary Histone deacetylase (HDAC) inhibitors generally fall into four recognized classes: (1) small molecular weight carboxylates; (2) hydroxamic acids; (3) benzamides; and (4) cyclic peptides. Exemplary HDAC inhibitors that can be used include: valproic acid, butyrate, phenyl-butyrate, pivaloyloxymethyl butyrate, trapoxin A, oxamflatin, depudepsin, depsipeptide (romidepsin, Istodax) and trichostatin A, the hydroxamic acids: belinostat (PDX101), LAQ824, and panobinostat (LBH589), the benzamides: entinostat (MS-27-275), C1994, and MGCD0103 (mocetinostat), 5 NOX-275 (also known as MS-275; Syndax Pharmaceutical Inc.) which is a synthetic benzamide derivative, as well as the compounds shown below combinations thereof and the like.

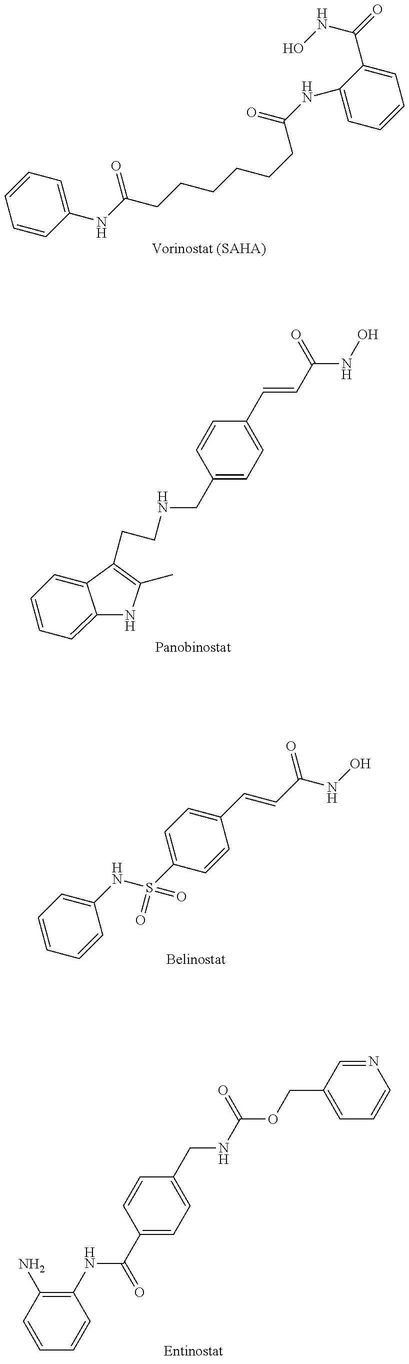 [SMILES: O=C(CCCCCCC(=O)Nc1ccccc1C(=O)NO)Nc1ccccc1 ; Cc1[nH]c2ccccc2c1CCNCc1ccc(/C=C/C(=O)NO)cc1 ; O=C(/C=C/c1ccc(S(=O)(=O)Nc2ccccc2)cc1)NO ; Nc1ccccc1NC(=O)c1ccc(CNC(=O)OCc2cccnc2)cc1]

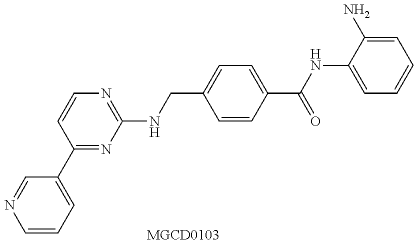 [SMILES: Nc1ccccc1NC(=O)c1ccc(CNc2nccc(-c3cccnc3)n2)cc1]

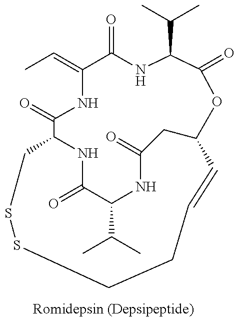 [SMILES: C/C=C1\NC(=O)[C@H]2CSSCC/C=C/[C@H](CC(=O)N[C@H](C(C)C)C(=O)N2)OC(=O)[C@H](C(C)C)NC1=O]

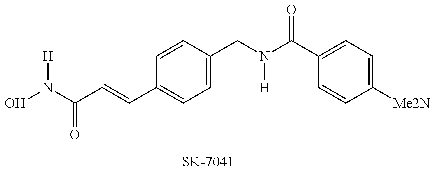 [SMILES: CN(C)c1ccc(C(=O)NCc2ccc(/C=C/C(=O)NO)cc2)cc1]

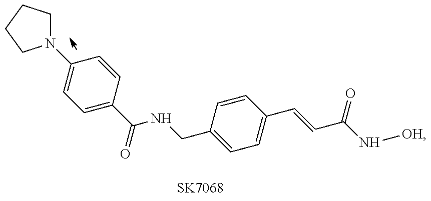 [SMILES: O=C(/C=C/c1ccc(CNC(=O)c2ccc(N3CCCC3)cc2)cc1)NO]

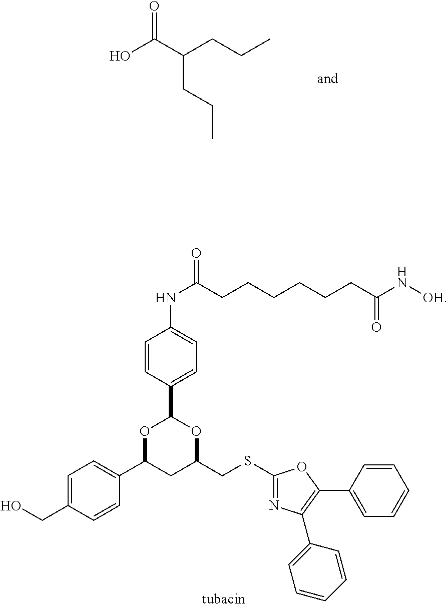

### The Effects of Maternal Exposures During Pregnancy on Offspring's Mammary Cancer Risk are Mediated Via Epigenetic Inheritance.

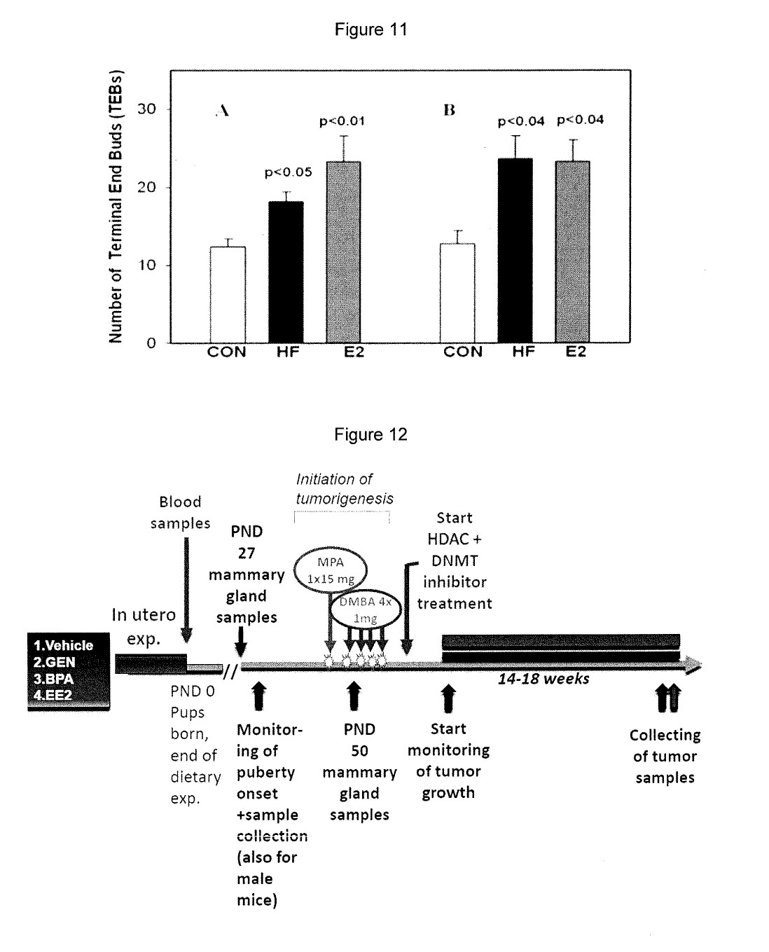

We have investigated whether maternal E2 exposure during pregnancy, or feeding pregnant dams a high fat diet that elevates pregnancy E2 levels (4, 64), affects mammary gland development in the 1st and 2nd generation offspring. Mammary glands of in utero E2-exposed rats exhibit a marked increase in the number of terminal end buds (TEBs). TEBs are the sites which give rise to malignant mammary tumors in rats; similar structures in the human breast—terminal ductal lobular units—give rise to 90% of human breast cancers. FIG. 11 shows that the number of TEBs is increased in the 1st generation of offspring of dams fed E2 or a high fat diet during pregnancy. Remarkably, the 2nd generation also exhibits the same change, although only their “grand-mothers” were exposed to E2 or a high fat diet during pregnancy; the 2nd generation has never been directly exposed to either one of the two manipulations. These preliminary findings provide convincing evidence to suggest that some breast cancers can be inherited through epigenetic mechanisms.

### Significance.

No genetic mutations have been identified in ˜50% of women who develop breast cancer and exhibit high family history of this disease. The preliminary data presented above show that the risk to develop mammary cancer can be elevated from generation to generation, a process that does not require DNA mutations but could be epigenetically inherited. One important factor leading to these inheritable epigenetic changes is an exposure to endocrine disruptors early in life. Since epigenetic changes are reversible, some inherited breast cancers are believed to be preventable by reversing these epigenetic changes—these modifications are likely to include dietary exposures which have been reported to methylate or re-activate epigenetically regulated genes.

### DEFINITIONS

The following definitions are provided for specific terms which are used in the following written description.

As used herein, the singular form “a”, “an” and “the” include plural references unless the context clearly dictates otherwise. For example, the term “a cell” includes a plurality of cells, including mixtures thereof.

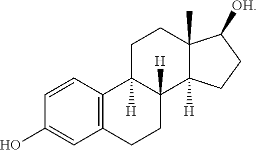

E2, also referred to as estradiol, has the structure: H

EE2, also referred to as ethinyl estradiol, has the structure:

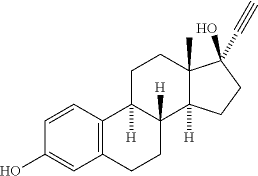

Genistein, also referred to as GEN, has the structure:

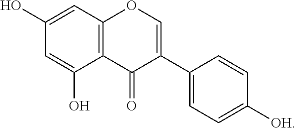 [SMILES: O=c1c(-c2ccc(O)cc2)coc2cc(O)cc(O)c12]

Valproic acid has the structure:

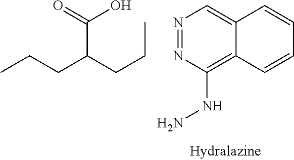

MicroRNAs (miRNAs) are short ribonucleic acid (RNA) molecules, on average only 22 nucleotides long. miRNAs are post-transcriptional regulators that bind to complementary sequences on target messenger RNA transcripts (mRNAs), usually resulting in translational repression and gene silencing

The transcriptome is the set of all RNA molecules, including mRNA, rRNA, tRNA, and other non-coding RNA produced in one or a population of cells.

DNA (cytosine-5)-methyltransferase 1 is an enzyme that in humans is encoded by the DNMT1 gene. DNA (cytosine-5-)-methyltransferase 1 has a role in the establishment and regulation of tissue-specific patterns of methylated cytosine residues. Aberrant methylation patterns are associated with certain human tumors and developmental abnormalities.

Epigenetics is the inheritance of changes in gene expression that does not involve changes in the DNA sequence, and include DNA methylation, histone modifications and non-coding microRNAs.

Breast cancer is the most common cancer in women, affecting 1 out of every 8 women in the US during their life time. However, etiology of breast cancer remains largely unconfirmed, as this disease in most women can only partially be explained by either familial or reproductive risk factors. However, hormones are clearly implicated in the etiology of breast cancer. Attempts to link adult exposure to EDCs to breast cancer have been conflicting (89). This reflects a possibility that adult exposure to EDCs has no significant effects on the breast while EDCs are harmful if exposed during fetal period (16). For example, in utero exposure to BPA (90) and genistein (2, 3) increases susceptibility to the development of mammary tumors in various animal models. Animal studies also indicate that maternal exposure during pregnancy to excess E2 or EE2 (synthetic estrogen, like DES is) increases mammary cancer risk in female offspring (4).

The precise mechanisms of developmental programming of cancer susceptibility by in utero estrogenic exposures remain to be established. According to Dr. Gail Prins (91), in utero estrogenic exposures leave a permanent mark into estrogen-sensitive cells that they once were exposed to excess estrogens. These cells may be more sensitive to the second hit that initiate (e.g., carcinogens and radiation) or promote (increased endogeneous hormones, e.g. due to overweight, or exogenous EDCs) cancer. It has become increasingly clear that epigenetic processes can play a central role in forming the memory mark.

Endocrine disruption refers to the interference of endocrine system functions by environmental chemicals. The endocrine system participates in many important functions of an organism, such as sexual differentiation before birth, sexual maturation during puberty, reproduction in adulthood, growth, metabolism, digestion, cardiovascular and immune functions, and excretion. Hormones are implicated in the etiology of certain cancers of hormone-dependent tissues such as those of the breast, uterus, and prostate gland. Environmentally released man-made chemicals are suspected of being responsible for numerous adverse effects on the endocrine function in wildlife species as well as in humans. Data generated in animal models indicate that in utero exposure to DES (35), BPA (36), and GEN (2, 3) increase susceptibility to the development of mammary tumors in various animal models. Further, it is known that the synthetic estrogen, DES (37), and BPA activate estrogen receptor alpha (ERα) (38), and ERRα (39) and ERRγ (34, 40-42). Additionally, GEN preferentially binds to the estrogen receptor beta (ERβ) (43). These receptors may play an important role in human breast cancers by increasing cell proliferation (44), by controlling metabolic genes (45, 46), insulin sensitivity (47, 48) or by inducing tamoxifen resistance (49). Creating an adequate assessment of how exposure to EDCs affects the endocrine system is complex due to the diversity of the EDCs, which can have synergistic effects in addition to their additive, individual effects. Furthermore, the complexity of the endocrine system itself is another factor that must be taken into account.

EDCs may be harmful to human health following fetal exposure. This likely relates to epigenetic changes in gene methylation patterns occurring during gametogenesis and embryonic development. During these periods, most of our genes are demethylated, followed by remethylation; the timing and pattern of remethylation depends on the tissue lineage, intrauterine environment, and maternal nutrition and other exposures (50).

### Epigenetic Modulation of Gene Expression.

The mammary gland undergoes extensive programming during fetal life and then re-programming at puberty and pregnancy. During fetal development, epigenetic programming interprets the information in the genetic code by means that do no involve a change in DNA sequence (51). One common epigenetic alteration is methylation of cytosine in the 5′position in CpG dinucleotides in a gene's promoter region, resulting in silencing of gene expression. Another common form of epigenetic regulation involves modifications of histones that in turn regulate chromatin folding. The epigenetic modifications are then inherited in somatic daughter cells, so that cell identity is maintained throughout life. As mentioned above, the scheduled epigenetic programming can be modified by factors that influence the epigenome, such as steroid hormones (52-54), maternal intake of folic acid (55) or genistein (56). Further, epigenetic changes caused by maternal dietary exposures may program the mammary gland morphology, since methylation is proposed to be the mechanism in the mammary gland that coordinates mammary epithelial differentiation (57). Several genes have been identified whose expression is epigenetically altered in adult tissues in animals that have been exposed in utero to environmental contaminants, such as DES (54), GEN (29) or BPA (58).

### In Utero Hormonal Exposures and Breast Cancer Risk.

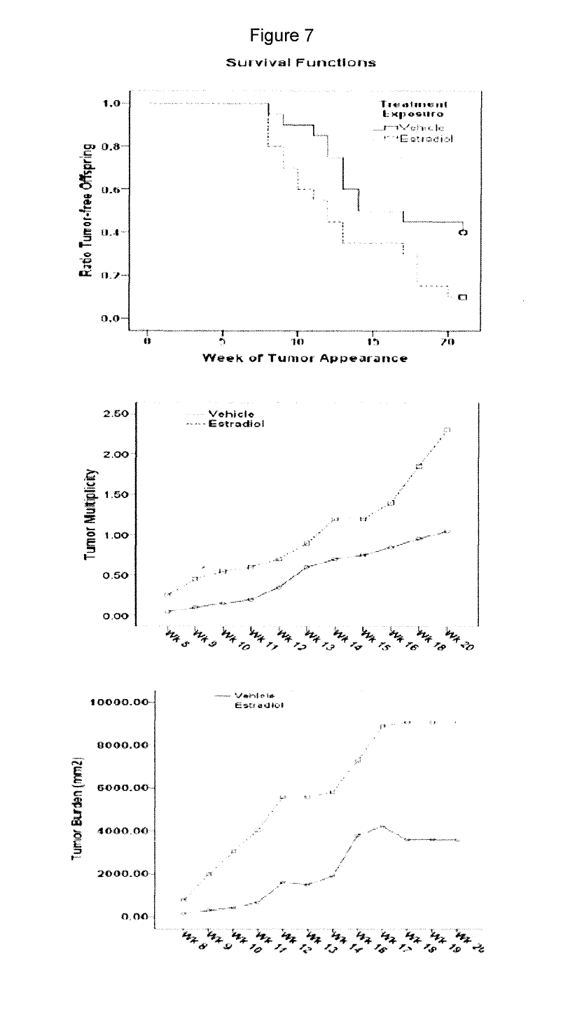

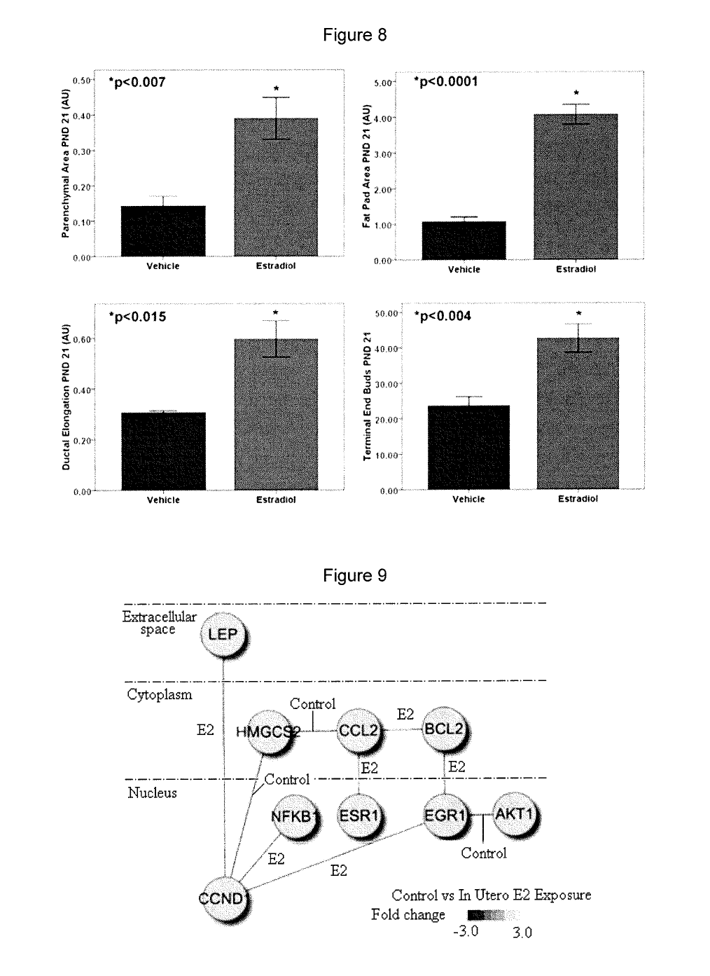

The level of estrogenicity of the in utero environment significantly affects the developmental programming of the mammary gland and its susceptibility to tumorigenesis later in life. Data from human studies strongly suggest (59), and animal studies show (4) (FIG. 7) that an elevated in utero estrogenic environment increases later susceptibility to develop breast cancer. The increase in mammary tumorigenesis is proceeded by alterations in mammary gland morphology, particularly in the number of terminal end buds (TEBs) (FIG. 8). TEBs are located at the tips of growing epithelial ducts and consist of a mass of body cells and a top layer of cap cells. They lead the growth of the mammary epithelial tree, and are the sites where the carcinogen-induced mammary cancers are initiated (60). Similar structures in a human breast, called terminal ductal lobular unit 1 (TDLU1), appear to be the sites of breast cancer initiation in most women (61). The reason why tumors arise from TEB/TDLU1 is not entirely clear, but can relate to increased cell proliferation in this structure (62) that is associated with increased levels of DNA adducts and reduced capacity to repair DNA damage (63). TEBs regress to terminal buds or differentiate to alveolar buds when the epithelial tree reaches the edges of the fat pad; that is about postnatal week 9 in rats (60).

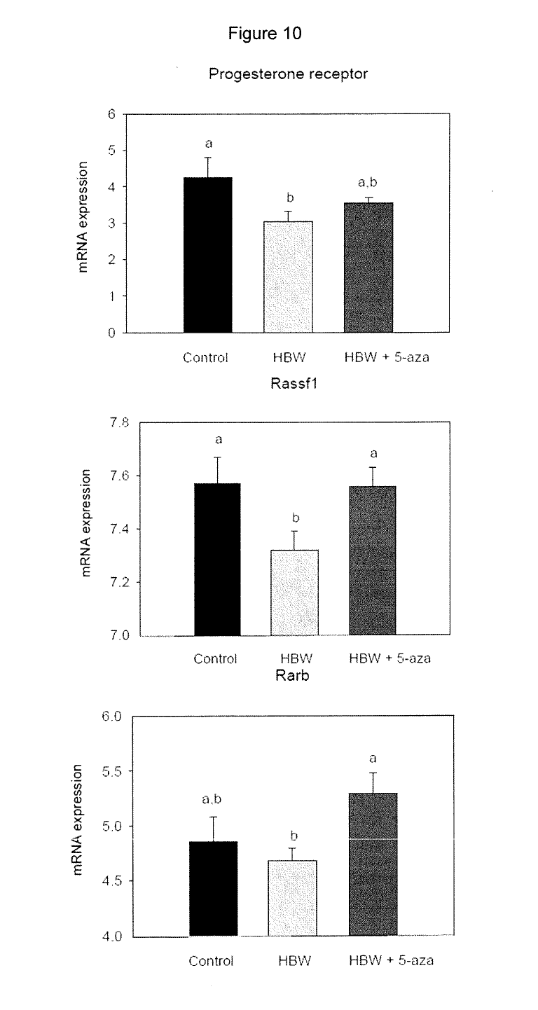

The key transcription factors and signaling that mediates the effects of in utero estrogenic environment on later estrogen sensitivity and breast cancer risk have been unknown. Transcriptome analyses, using innovative Differential Dependency Network (DDN) modeling algorithm, of mRNA from normal adult rat mammary glands exposed in utero to E2, or a high fat diet which elevates in utero E2 levels (and from controls), have identified several seed genes that can be useful in initiating predictive computational modeling (FIGS. 9 and 10). Since the exposure was in utero, but the differential transcriptome analysis was done in adulthood, the altered expression of several of these genes over time could be a consequence of transcriptional programming regulated by promoter methylation status. Many of these genes are known to be regulated by promoter methylation, e.g., RARB, p16 (INK4a/ARF), progesterone receptor (PR) and HIN-1. FIG. 9 shows that mammary glands of 2-month-old offspring exposed to a high fat diet in utero express lower levels of PR and Rassf1, and that these genes and also RARB are up-regulated in the high fat offspring which were treated twice with a methylating agent 5-aza before puberty onset at the age of 1 month. Importantly, these same genes are epigenetically regulated and differentially expressed in needle aspirate samples in women from non-BRCA1/2 breast cancer families (22). Although 5-aza is not suitable to be used in cancer prevention studies due to its toxicity, several other compounds, such as HDAC inhibitors, are currently available for clinical use. The resulting findings provide proof-of principle evidence that postnatal exposure to a demethylating agent can reverse changes in gene expression.

Natural 17β-estradiol (E2) contains three-ring phenanthrene; of which the first or A ring contains a phenolic hydroxyl group, which is required for estrogenicity. The pharmaceutical estrogens, DES and ethinyl estradiol (EE2), are as potent as the parent compound, E2, and they remain stable when consumed orally. Endocrine disrupting chemical (EDCs), in turn, are structurally diverse chemicals which function as estrogens and alter normal endocrine functions, although most of the EDCs have considerably weaker affinity to the estrogen receptor (ER) than E2 does. In addition, they generally interact with a variety of other receptors, enzymatic pathways involved in steroid hormone synthesis, and other mechanisms that impact endocrine and reproductive systems.

Pregnant mouse dams were exposed to either EE2 or endocrine disruptors genistein and BPA. They all are previously reported to increase mammary tumorigenesis among female offspring (2-4, 90). These exposures had some effects on early reproductive development, but the effects were different in the EE2 and genistein offspring. Consistent with a previous study, in utero exposure to EE2 led to earlier puberty onset (4). In utero genistein exposure, in turn, delayed puberty onset. This is in accordance with some studies, although most have reported accelerated vaginal opening following in utero genistein exposure (93-95). BPA had no effect on puberty onset. Since both EE2 and genistein exposed offspring exhibited increased mammary cancer risk, and BPA is likely to do the same when sufficient number of mice are included to the study, changes in puberty onset induced by EDCs do not appear to be causally linked to the development of breast cancer in mice.

Earlier studies have reported an increase in the number of targets for malignant transformation; i.e., TEBs, in offspring of dams exposed to estradiol (4, 72), genistein (93) or BPA (96). Findings obtained in this study confirm these previous observations and indicate that mammary gland is a target of in utero exposure to EDCs. Elevation of TEBs is therefore one biomarker of an exposure to EDCs in utero in mice and rats. Some studies have shown that abnormal gene expression in TDLUs is associated with increased breast cancer risk in women (97).

Epigenetic changes induced by altered endocrine environment during fetal development are proposed to mediate the effects of EDCs on later breast cancer risk. Unlike genetic changes that cannot be easily reversed, epigenetic changes can be reversed by inhibitors of histone deacetylase (HDAC) and/or DNA methyltransferase (DNMT) activities (36). Such inhibitors are being tested for the treatment of breast cancer and early data are promising (98). In a study, pregnant mice were exposed to either EE2, genistein or BPA during pregnancy, and their female offspring exhibited increased mammary tumorigenesis. The results show that an exposure to the HDAC inhibitor hydralazine and the DNMT inhibitor valproic acid in drinking water prevented the increase in mammary tumorigenesis in the offspring exposed to E2 or genistein in utero (FIGS. 3-5). These findings provide experimental evidence indicating that the increase in breast cancer risk induced by an exposure during fetal development to excessive estrogenic environment can be prevented or at least substantially preliminary by compounds which inhibit HDAC and/or DNMT. These results have two important implications: (1) in utero estrogenic exposures increase later breast cancer risk via epigenetic mechanisms, (2) increased breast cancer risk in EDC daughters may be reduced by HDAC and DNMT inhibitors, and, (3) women who are at increased risk developing breast cancer, or have a more aggressively growing breast cancer, due to their mothers' EDC exposure during pregnancy, and are currently under-diagnosed and under-treated, can be identified based on their DNA methylation and/or miRNA signatures.

In exemplary embodiments, to prevent or substantially eliminate breast cancer in women exposed to EDCs in utero, a means is employed to identify them when these women are adults. Excessive levels of estrogens during fetal development might leave an epigenetic imprint in the DNA, which causes methylation of normally unmethylated CpG islands. This imprint can be detected not only in the organs where cancer develops, but also in unrelated target tissues, particularly if the stem cells where the epigenetic event occurred during fetal development were present in “cancer” organs and other target tissues. Women exhibiting methylation of these genes are then prime targets of intervention using existing epigenetic compounds.

Another marker of in utero EDC exposure could be down-regulation of non-coding miRNAs. miRNAs are short non-coding single stranded RNAs composed of approximately 21-22 nucleotides that regulate gene expression by sequence-specific base-pairing with the 3′-untranslated region (3′UTR) of target mRNAs to induce their post-transcriptional repression (7), either by inducing inhibition of protein translation or degradation of the mRNA. Several miRNAs have been identified which are regulated by estrogens and which are related to breast cancer (8, 9). The mechanisms involved are not known but may include inhibition of maturation of miRNA from their pri-miRNAs via Drosha and Dicer (10-12). Remarkably, miRNAs which in our preliminary study were consistently down-regulated in an adult mammary gland in rats exposed to EE2 or high fat diet in utero compared to control mammary glands (FIG. 28), were all the same miRNAs which have been reported to be down-regulated by E2 in MCF-7 human breast cancer cells (13). Some of these E2-suppressed miRNAs that have been identified in MCF-7 cells and adult mammary glands of in utero EE2 exposed rats, target enzymes that induce and maintain DNA methylation (DNA methyltansferases: DNMT1, DNMT3a, DNMT3b); these are called epi-miRNAs. They include miR-148 and miR-152 which target DNMT1, miR-194 targeting DNMT3a, and miR-26a/b targeting DNMT3b (14, 15) (TargetScan 5.2).

These findings suggest that high in utero estrogenic environment, by initially down-regulating miRNAs which then leads to up-regulation of genes involved in inducing DNA methylation (DNMTs), may explain methylation of PcTGs and TSGs (see Tables 1 and 2). Further, since many miRNAs can be methylated (17-20) and they also regulate the expression of genes involved in the methylation; i.e., DNMTs (13), it is possible that changes in their expression are involved in mediating the effects of maternal estrogenic environment during pregnancy on offspring's mammary tumorigenesis.

It is to be understood that this application is not limited to particular embodiments described. It is also to be understood that the terminology used herein is for the purpose of describing particular embodiments only, and is not intended to be limiting, since the scope of the present application will be limited only by the appended claims.

Unless defined otherwise, all technical and scientific terms used herein have the same meaning as commonly understood by one of ordinary skill in the art. Although any methods and materials similar or equivalent to those described herein can also be used in the practice or testing of exemplary embodiments, specific preferred methods and materials are now described.

### EXAMPLES

### Example 1

Determination of an increased risk in mammary cancer in animals exposed to excessive levels of EDCs in utero estrogenic environment which is caused by epigenetic changes, and that the increased risk can be reversed by erasing those epigenetic changes in adulthood by treatment with DNMT and HDAC inhibitors.

### Animals and Diets.

Time pregnant C57BL/6 female mice were obtained from Harlan (Harlan-Teclad, USA) on gestation day 6, and were randomly assigned into three groups on the following day (gestation day 7). The basic diet formulation in the study was a semi-purified AIN93G (the American Institute of Nutrition) modified to replace soy oil for corn oil; all the diets of the three treatment groups were also purchased from Harlan. The diets were isocaloric, as ethinyl estradiol (EE2) or genistein (GEN) were added in the diet at the expense of comstarch. Additions to the basic diet were as follows: 1) EE2 0.1 ppm, and 2) GEN 500 ppm. EE2 was purchased from Sigma Chemicals (St. Louis, Mo., USA), and GEN was a gift from Dr. William Helferich (University of Illinois, Urbana, Ill., USA).

The pregnant dams were fed these diets until the day of the delivery of their pups (Post Natal Day 0, PND 0). Starting on PND 0, all mice were fed the control diet until the end of the study. Female mice born from the dams fed with the different above mentioned diets were used in the subsequent studies. For study outline, see FIG. 12. Animals were housed in a temperature- and humidity-controlled room under a 12-hour light-dark cycle. All animal procedures were approved by the Georgetown University Animal Care and Use Committee, and the experiments were performed following the National Institutes of Health guidelines for the proper and humane use of animals in biomedical research.

### Induction of Mammary Tumors.

Mammary tumors were induced by administration of a subcutaneous injection of Medroxyprogesterone Acetate (MPA) to mice at 6 weeks of age, followed by administration of 1 mg 7,12-dimethylbenz(a)anthracene (DMBA) (Sigma, St. Louis, Mo.) weekly for four weeks (weeks 7, 8, 9, 10) (23). The carcinogen was dissolved in corn oil and administered by oral gavage in a volume of ca. 0.1 ml.

One week after the last dose of DMBA was administered, some of the mice were started on hydralazine (5 mg/kg/day) and valproic acid treatments (1.16 g/kg/day) treatments.

The two compounds were given in a drinking water, which was prepared fresh twice a week. The doses were chosen based on the literature (24-26). The number of animals in each group is as follows: Control n=18, Control given HDAC+DNMT inhibitors n=9, EE2 n=9, EE2 given HDAC+DNMT inhibitors n=6, GEN n=14, and GEN given HDAC+DNMT inhibitors n=8.

### Monitoring Mammary Tumorigenesis.

Mice were examined for mammary tumors by palpation once per week. Tumor growth was monitored for 16 weeks after the last dose of DMBA administration. During the follow-up, animals in which tumor burden approximated 10% of total body weight were euthanized, as required by the ethical guidelines of our institution. All surviving animals were sacrificed at that point for sample collection. The end-points for tumor data analysis were (i) latency to tumor appearance, (ii) the number of animals with tumors (tumor incidence), and (iii) the cumulative tumor area (tumor burden). The latter was achieved by adding the total tumor area per group each week and dividing it with the number of mice in the group.

Effect of DNMT1: RNA Extraction, cDNA Synthesis and Quantitative Real-Time PCR (qRT-PCR) Analysis.

Total RNA was extracted using an RNeasy lipid mini kit (Qiagen, CA), according to manufacturer's instructions. One lag of total RNA per sample was used as a template for oligo-dT primed cDNA synthesis with a Supterscript reverse transcriptase, using Taqman Reverse Transcription kit (Applied Biosystems, CA). The cDNA samples served then as templates for quantitative real time PCR analysis with specific primers for the target gene using QuantiTect SYBR green PCR kit (Qiagen Inc., Valencia, Calif.) and an ABI Prism 7900 Sequence Detection System. Each sample was run in triplicate on a 384-well plate, and the qPCR run was repeated twice. The Ct values were averaged from the triplicates for each sample. To normalize the target gene expression the ratio to control RNA was calculated, i.e. the averages of the Ct triplicates from the Dnmt1 gene were divided by the average of the triplicates from the 18S gene. To determine the fold change of the target gene expression the results are presented as 2^{ΔCt}, where ΔCt=CT^{target}−Ct^{18S}.

Dnmt1 primers were as follows: mDnmt1-Forward: AGACCAGGATAAGAAACGCAG, mDnmt1-Reverse: TTTCCGCCTCAATGATAGCT, giving an amplicon of 175 bp. The primer sequences for m18S rRNA were as previously published by Wang et al. (27).

### Statistical Analysis.

Mammary tumor latency was compared with the final tumor burden in utero EE2 and GEN exposed mice to control mice, and in utero EE2 and GEN exposed mice to control mice exposed to HDAC+DNMT inhibitors separately; analysis was done using one-way ANOVA. Cumulative tumor burden during 16 weeks of tumor monitoring in the three groups, separately in those mice not given HDAC+DNMT inhibitors and those treated with the epigenetic drugs, was analysed using repeated measures one-way ANOVA. Log rank analysis was performed to compare mammary tumor incidence in the control and in utero EE2 and GEN exposed mice, again separately in those mice not given HDAC+DNMT inhibitors and those treated with the epigenetic drugs. Data for changes in DNMT1 expression among the groups in the mammary glands and tumors were assessed using two-way ANOVA, followed by Fisher LSD test

### Results

### Mammary Tumorigenesis

Tumor Latency.

Latency for first tumor to develop was significantly shorter in mice exposed to EE2 or genistein in utero, compared to the vehicle treated control mice (p=0.038); the difference was most significant between the control and genistein groups (p=0.012) (FIG. 3a). However, if the mice were treated with HDAC+DNMT inhibitors, no significant differences in tumor latency among the three groups were seen (p=0.908) (FIG. 3b). In fact, they reversed the shortening in latency in the genistein group (p=0.018). Similar tendency was seen in the EE2 group, but perhaps reflecting low number of mice in this group, the difference did not reach statistical significance. In contrast, in the control group no changes in tumor latency were caused by epigenetic compounds.

Tumor Incidence.

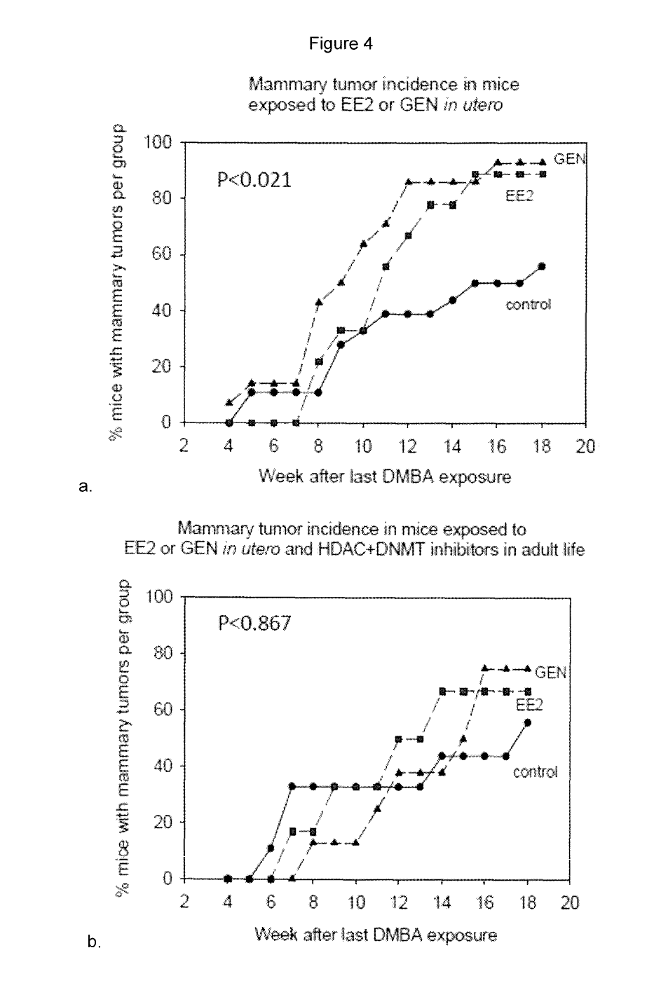

Mammary tumor incidence was significantly higher in mice exposed to EE2 or genistein in utero than in the control mice (p=0.021), as previously reported in rats exposed to E2 (4) or genistein (2) during fetal period (FIG. 4a). The difference was more significant in the genistein group (p=0.009) than in the EE2 group (p=0.09). Treatment of the mice exposed to EE2 or genistein in utero with HDAC+DNMT inhibitors in adult life abolished this difference, compared to the HDAC+DNMT inhibitor treated control mice (p=0.867) (FIG. 4b).

Tumor Multiplicity.

No differences in mammary tumor multiplicity among the groups were seen. Most mice developed only one mammary tumor, regardless of in utero exposure (p=0.657 in the non-epi drug groups). Since tumor multiplicity assessment included only those mice that developed one or more tumors (mice without tumors were not included to the analysis), it was not possible to detect a reduction in multiplicity among the mice treated with HDAC+DNMT inhibitors. Incidence was not increased either in mice treated with the epi drugs, compared to mice not given HDAC+DNMT inhibitors (data not shown).

Tumor Burden.

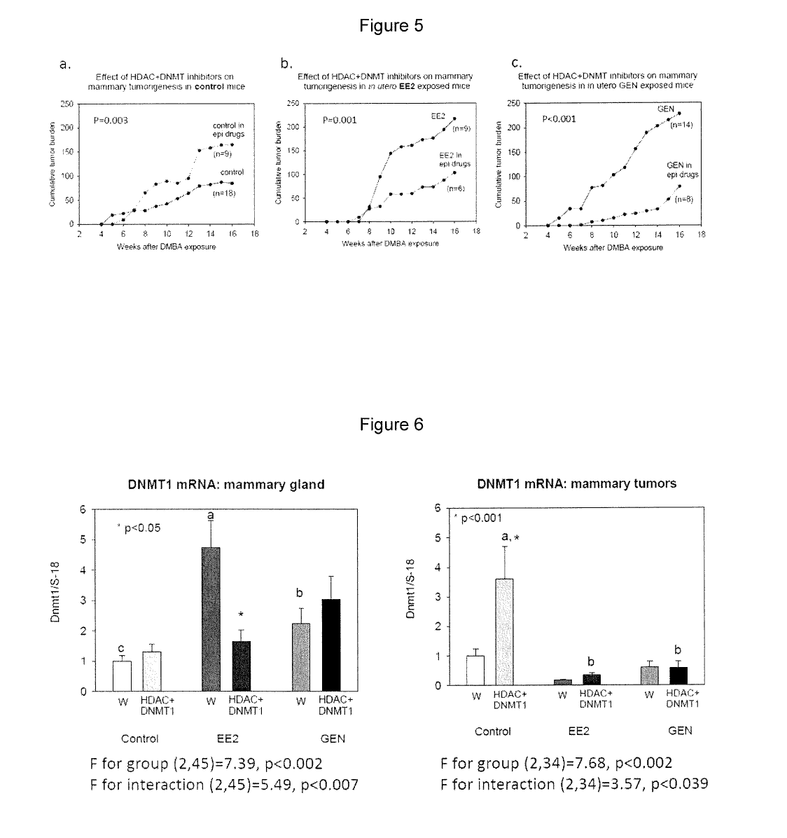

Differences in cumulative tumor burden (size of all tumors per mouse) were assessed by determining total weekly tumor burden in the group, divided by the number of mice in the group. Final tumor burden per mouse was significantly higher in both the EE2 (p=0.039) and genistein (p=0.009) groups, compared to the controls (p=0.018). This difference was not seen among the three groups of mice treated with HDAC+DNMT inhibitors in adult life (p=0.674). To illustrate the ability of the HCAC+DNMT inhibitors to reverse the increase in mammary tumorigenesis, cumulative tumor burden was compared within each in utero exposure group which were not treated or were treated with epigenetic drugs by using repeated measures ANOVA. FIG. 5a shows that in the control group, HDAC+DNMT inhibitors increased mammary tumor burden (p=0.003), but in the EE2 (p=0.001) (FIG. 5b) and genistein groups (p<0.001) (FIG. 5c), a highly significant reduction in cumulative tumor burden was seen.

### DNMT1 Expression.

Expression of DNMT1 was determined both in the mammary glands and tumors in mice exposed to EE2 or GEN in utero. As previously seen in rats (see FIG. 2), mice exposed to EE2 in utero exhibited an increase in the expression of DNMT1 (p<0.002), and GEN exposed offspring showed a similar increase (p<0.004) (FIG. 6a). An exposure to HDAC+DNMT1 inhibitors reversed the increase in the EE2 group (p<0.004), but not in the GEN group (F for interaction: p<0.007).

In the mammary tumors, DNMT1 expression was significantly lower in the mice exposed either EE2 (p<0.002) or GEN in utero (p<0.004) than in the control mice. Further, HDAC+DNMT inhibitors significantly increased DNMT1 expression in the mammary tumors in the control mice (p<0.001), but not in the EE2 or GEN group (F for interaction, p<0.039) (FIG. 6b).

### Example 2

This example is designed to assess and compare the effects of in utero exposures through a pregnant mouse dam to estradiol (E2), the synthetic estrogen diethylstilbestrol (DES), genistein (GEN), and bisphenol A (BPA) on genome wide methylation patterns in the offspring's mammary gland and peripheral DNA.

### Exposures to Endocrine Disruptors.

Fetal exposures to E2 (4), DES (30), GEN (2) and BPA (31) have been shown to increase the risk of mammary cancer later in life. C57BL/6 mice are utilized in these studies, since they are sensitive to the effects of endocrine disruptors. Mice are mated by housing two females with a male, and gestation day 1 will be determined by checking positive plugs daily. Exposures to the endocrine disruptors occur daily via the diet during gestational days 7 and 20. Mice deliver on gestation day 21, but if estrogen levels remain high after parturition, dams have been reported to fail to exhibit proper maternal behavior. For that reason, all pregnant dams will be switched to a standard AIN93G diet on gestation day 20.

Pregnant mice are exposed to E2, DES, GEN or BPA at the doses previously found to increase mammary cancer among offspring. The exposure occur orally via AIN93G semipurified laboratory chow and the following doses are utilized: 0.05 or 0.5 ppm ethinyl estradiol, 100 or 1,000 ppb DES, 100 or 500 ppm genistein, and 0.25 and 25 ppm BPA. The effects of the two doses of each compound on mammary gland morphology among the offspring and expression of genes which might be targets of epigenetic regulation are assessed. One dose per endocrine disruptor is selected to determine global methylation patterns.

For the global methylation arrays, and determining changes in gene expression and mammary gland morphology, a total of 10 female offspring are needed per group. To obtain 20 female offspring, 6 pregnant dams are needed. Since a total of 9 exposures are implemented during pregnancy (control group and 2 doses per endocrine disruptor, for four different disruptors), 54 pregnant dams are used. In addition, 6 pregnant dams per group are used for determining the levels of endocrine disruptors in the dam. Thus, a total of 108 pregnant mice are needed.

### Determining Endocrine Disruptor Levels in the Blood or Urine.

Blood for determining the exposure levels of various endocrine disruptors is collected from 6 pregnant dams on gestation day 19. Collection is done by cardiac puncture to be able to obtain sufficient quantity of serum for determining the levels of E2, DES, and GEN. Urine is also be collected at sacrifice directly from the bladder for measuring BPA levels. Blood and urine samples are processed as previously described (65), and the levels of E2, DES, GEN and BPA are measured.

### Tissue Sampling.

Mammary glands from control and experimental mice are obtained on postnatal day (PND) 21 and PND 50. At each time point, 10 mice per group are sacrificed, and on PND 50 at diestrus stage (most common estrus stage in developing mice). A total of 180 mice are needed to have 10 mice per group at two time points (PND 21 and 50)×5 exposures (vehicle, E2, DES, GEN and BPA exposures)× and 2 doses (for experimental groups). Blood is also collected, and lymphocytes are separated and used to obtain peripheral DNA. Mammary glands obtained from the mice sacrificed at the two different developmental time points are used as follows: right 4th gland—DNA; left 4th gland—mammary wholemount; right 2-3rd gland: tissue blocks for protein levels in IHC/cell proliferation and protein for Western blots; and left 2-3rd gland—RT-PCR for mRNA assays.

### Mammary Gland Morphology.

Mammary gland morphology might be indicative of changes in susceptibility to develop mammary cancer. It has been found that increased risk of developing mammary tumors is proceeded by an increase in the number of terminal end buds (TEBs) (32, 66, 67). Other changes might also correlate with the risk, such as epithelial elongation, epithelial density and fat pad size.

Visual Examination of the Mammary Wholemounts.

Analysis of mammary epithelial structures in whole mounts is based on visual evaluation and computer-assisted image analysis. A visual scale (68) is used to assess growth patterns of mammary epithelial cells. In the scale-method, the stained mammary whole mounts are examined under an Olympus dissecting scope. The following characteristics of the coded mammary glands are evaluated double-blindly using a 5-point scale for a density of (0: no structures detected, 5: numerous structures detected: (i) epithelial ducts; (ii) lobulo-alveolar structures, and (iii) ductal branching. Since estrus stage may affect the mammary gland morphology and proliferation in mice, animals are sacrificed at the estrus. The actual number of TEBs are counted.

Visual Imaging Technique.

Ductal elongation is determined by measuring the distance from the nipple to the tip of the parenchymal area. The parenchymal area is analyzed by digitizing a mammary wholemount using a 20× objective (BX40; Olympus Optical, Tokyo, Japan) via the microcomputer imaging device (MCID) computer imaging analysis system (Imaging Research, St. Catharines, Ontario, Canada).

### Gene Expression.

Since global methylation arrays cannot be done to all mice due to high costs of the assays and limited funding available for the study, one dose per exposure and age (either PND 21 or 50) is selected. The dose is selectec based on two factors: dose causing most morphological and gene expression changes, and age when these changes are most prominent. Gene expression for RARB (M3 and M4), ESR1, INK4a/ARF, BRCA1, PRA, PRB, RASSF1A, HIN-1, and CRBP1 (22) is determined. The Mouse Wnt Signaling Pathway PCR Array from SABiosciences (Frederick, Md.), which contains myc, is used to study changes in the pathways regulating cell proliferation and differentiation. The PCR array contains optimized real-time PCR primer assays on 96-well plate for genes in Wnt/beta-catening pathway. The array provides expression analysis at the real-time PCR sensitivity level and multi-gene profiling capacity of a microarray. When using this array, total RNA is prepared using RNeasy Mini-kit according to the manufacturers' protocols to prepare cDNA template. Equal volumes of the cDNA to RTqPCR Mater Mix and Aliquot Micture are added across PCR arrays, and then run in Real-Time PCR Instrument. Web based PCR array data analysis is performed using a software provided by SABiosciences.

### Proliferation.

Cell proliferation in the TEBs, and ductal and alveolar structures at the two different ages is assessed by immunohistochemistry using Ki67 (SP6, BioGenex Laboratories) antibody. Proliferation index is determined by calculating the percentage of cells positive for Ki67 staining among 1,000 cells per structure. All sections are blindly evaluated with help of the Image Tool software. In addition, cell proliferation is determined by injecting BrdU i.p. at a dose of 70 mg/kg, and mice are sacrificed after 2 hr. Tissue is removed and fixed in formalin to prepare paraffin sections, which is then deparaffinized, incubated in 0.1% trypsin for antigen retrieval, Incubated in 3% goat serum in PBS/Tween-20 for 10 min, and incubated with a mouse monoclonal BrdU antibody or nonimmune mouse IgG (1:300 dilution) overnight at 4° C. Visualization is carried out with DAB staining.

### Global Methylation Assessment and Validation.

Changes in methylation patterns is determined in the DNA from mammary glands and lymphocytes obtained from 21- or 50-day-old offspring. As indicated above, only one dose per endocrine disruptor is used, and DNA is obtained from 6 mice per group. These changes are compared to mammary gland morphology among the exposure groups to determine whether morphological changes can serve as biomarkers of epigenetic changes. Morphological changes, particularly an increase in terminal end bud number caused by in utero exposure to endocrine disruptors, are predictive of changes in mammary cancer risk (32).

Alteration in the methylation status of DNA elements can have as consequence major changes in the gene expression pattern or genomic instability, thus contributing to cancer. Site-specific cytosine methylation (5-me) of CpG dinucleotides is the most intensively investigated epigenetic modification in cancer (69, 70).

Recently, several genome wide methylation profiling techniques have been developed to screen the level of methylation changes in abnormal cells. Traditionally, methylation sensitive restriction enzymes have been used to distinguish methylated from unmethylated DNA. Using restriction enzyme-based approaches with DNA array technologies provides the ability to identify in a high-throughput manner, hypermethylated DNA regions in cancer cells (71). However, restriction enzymes-based methods are limited by the distribution of their recognition site. Another popular approach in assessing methylation at individual loci is the conversion of unmethylated cytosine with sodium bisulfite followed by sequencing. This method is quite laborious and cannot be easily applied to screening a large set of samples or sequences. To circumvent these issues, an antibody specific for methylated cytosine was used in a chromosome-wide analysis to immunoprecipitate methylated DNA (72). As reported by Zilberman et al., investigating the role of DNA methylation in Arabidopsis thaliana, this approach showed better outcome compared to the restriction enzyme based strategy (73). All these approaches have been incorporated in high-throughput genome wide methylation screening assays by biotech companies, all having specific biases and limitations though (71).

The Mouse and Human CpG Island Microarray Kits (both from Agilent Technologies, Santa Clara Calif.), which are based on the methyl DNA immunoprecipitation (mDIP) method described by Weber et al. (72) are used. The mouse array contains 97,652 oligonucleotide probes for 16,030 CpG islands. The human array contains 237000 oligonucleotide probes for 27,800 CpG islands covering virtually all human transcripts as well as several intergenic sequences. The method uses an antibody against 5-methyl-cytosine to enrich for regions of methylated DNA. Relative methylation levels within a given sample are analyzed by generating mDIP-enriched DNA, which is then labeled and competitively hybridized against labeled input genomic DNA on a microarray. Methylation-enriched and methylation-depleted regions are labeled with different fluorescent dyes, enabling the calculation of the ratio of log transformed fluorescent signal of methylated and unmethylated sequences as a red-out of methylation level. A positive log ratio indicates hypermethylation, while a negative log ratio indicates hypomethylation.

Employing antibodies against 5-methyl cytosine can side-step the limitations of restriction enzyme based technologies and the use of bisulphite treatment. The only drawback of this approach is the necessity to have relatively high amount of DNA, as the procedure requires 4 micrograms of input DNA.

The procedure begins with the extraction of DNA from fresh tissues, stored in −80 since harvest DNA from Tissues are digested in a solution of 1×TE (pH 7.5), 0.1% SDS, and proteinase K at 55 C for 2 days, and then extracted with 25:24:1 phenol/chloroform/isoamyl alcohol and precipitated with glycogen, 10 mol/L ammonium acetate, and 100% ethanol. The DNA quantity and quality is then assessed using a Thermo Scientific NanoDrop™ Spectrophotometer. Four micrograms of DNA are then processed according to manufacturer's instructions. Briefly, the procedure involves the preparation of magnetic beads by incubation with the anti 5-methyl cytosine antibody. The prepared beads are then incubated with 3.75 micrograms of the fragmented DNA, and then the IP-enriched DNA is eluted from the beads and extracted with 24:24:1 phenol/chloroform/isoamyl alcohol, precipitated with ethanol, glycogen and NaCl at −80 C, pelleted in a microcentrifuge, washed with 70% ethanol and dried in a speedvac. The IP-enriched DNA and 200 ng of input DNA are then labeled with Cyanine 3- and Cyanine 5-dUTP nucleotides, respectively, and hybridized on the microarray at 67 C for forty hours. The arrays are then scanned in the Agilent microarray scanner and analyzed with the Agilent Feature Extraction Software normalizing the Cy3 and Cy5 signals.

For validation of the microarray data, 40 samples up to 50 CpG islands each are assessed in triplicate by pyrosequencing using the Pyromark MD (Biotage, Sweden) instrument Pyrosequencing is a sequencing-by-synthesis method that quantitatively monitors the real-time incorporation of nucleotides through the enzymatic conversion of released pyrophosphate into a proportional light signal, thus enabling a quantitative measurement of the methylation extent at each CpG site. Pyrosequencing combines the ability of direct quantitative sequencing, reproducibility, speed and ease-of-use, being a flexible analysis platform that can be applied for the analysis of DNA methylation in CpG-rich regions as well as CpG-poor regions, where the number of required CpGs within the primer binding sites might be insufficient for alternative methods such as methylation-specific PCR approaches (76). The method consists in DNA treatment with sodium bisulfite in order to convert all un-methylated cytosine residues to uracil which is then converted to thymine during PCR reaction. A target region of up to 350 bp is then amplified by PCR using a pair of primers complementary to the bisulfite-treated DNA sequence, amplifying all states irrespective of methylation status. One of these two amplification primers carries a biotin label at its 5′-terminus. The incorporated biotinylated primer is subsequently immobilized on streptavidin-coated beads used to purify and render the PCR product single-stranded (as only one strand is biotinylated). A pyrosequencing primer complementary to the single-stranded template is then hybridized to the template, and the pyrosequencing reaction is performed by the sequential addition of single nucleotides in a predefined order. The data are then analyzed using the Pyro Q-CpG™ Software (Biotage, Sweden) by calculating the percent methylation for each CpG island in the sequence context

### Data Analysis.

Our motif-directed network component analysis (mNCA) can be used for analysis of the transcription factors and their target genes (particularly useful for finding VDR-regulated genes). This approach applies a novel method for extracting motif data to provide an initial topology, i.e., the transcription factor and the edges connecting it to its candidate target genes, and apply a stability analysis to test the robustness of their predicted connecting edge. To identify genes associated with signaling activities, our differential dependency network analysis (DDN), which extracts local dependency models by decomposing the entire observed network (molecular profile) into a series of local networks (modules) represented by a set of conditional probabilities inferred from linear regression models is used. Statistically significant topological structures and “hot spots” within the local networks/modules are detected. Subsets of genes most likely to cooperate to perform a specific biological function, e.g., proliferation, are identified by applying a simple gene ontology analysis to identify genes associated with this function and then apply our non-negative independent component analysis (nICA). The nICA method exploits the non-negative nature of gene expression and allows us to explore multiple projections to find gene modules (81). Expression of methylated genes will be analyzed using a panel of novel bioinformatics tools developed by Wang et al (79, 82, 83).

### Statistical Analysis Plan.

Based on our previous study, the number of animals per group (n=6-10) provides sufficient power to identify changes in mammary gland morphology, gene expression and gene methylation patterns. To avoid a litter effect, mice sacrificed on PND 21 and 50 will be obtained from all litters available (n=6 litters per exposure). Changes among the groups are first compared separately on PND 21 and 50 by ANOVA. Data failing normality and equal variance tests, will be log transformed and/or corresponding non-parametric tests will be used. The mammary epithelial and fat pad growth will be measured metrically, and other measures of mammary whole mount morphology, and cell proliferation data also are metric, and therefore data will be analysed using ANOVA. Tukey test will be used to determine individual differences among the variables.

These altered genes can be used in a unique screen to predict endocrine disruption by novel compounds and ultimately as a tool to understand and predict reproductive (breast) cancer risk in humans. These altered genes can also be used in assessing whether the such genes are modulated by epigenetic means. The information generated can be used to regulate in utero exposure to new drugs and food additives with endocrine activity.

### Example 3

Determine if the epigenetic changes identified in global methylation arrays are causally linked to an increased susceptibility to developing mammary cancer by utilizing compounds which reverse methylation changes.

### Exposures to Endocrine Disruptors.

Pregnant dams are exposed to control diet or E2, DES, GEN or BPA. Only one dose per endocrine disruptor are used as described in Example 2. Further, if one or more of these compounds do not generate epigenetic changes in the mammary glands, it will not be studied here. To generate 40 female offspring, 15 pregnant dams per group are exposed to the endocrine disruptors. Thus, a total of 75 pregnant dams are required.

### Administration of Methylation and HDAC Inhibitors.

The changes in methylation patterns detected in Example 2 can be reversed by demethylation agents. A study is conducted by treating mice with inhibitors of DNA methylation and histone deacetylases (HDACs). These include DNA methylation inhibitor hydralazine and the histone deacetylase inhibitor valproic acid (84), two drugs which are used to treat for cardiovascular and neurological conditions, respectively. Data by Dueñas-González et al. (33) indicate that this approach is effective in treating locally advanced breast cancer. One half of the offspring in each treatment group receive vehicle and the other half 5 mg/kg hydralazine and 4 mg valproic acid daily by intraperitoneal route. The administration is started after mammary tumors are initiated but before tumors are detected; i.e. one week after the final carcinogen dose is administered, and continue until the end of the tumor monitoring period.

### Mammary Tumorigenesis.

Mammary tumors are induced by pre-treating 6-week-old mice with 15 mg/kg medroxyprogesterone acetate treatment (MPA) and one week later, by administering 1 mg 7,12-dimethylbenz[a]anthracene (DMBA) for 4 times, at one week intervals. DMBA also is diluted in oil, but it is administered by oral cavage, in a volume of 0.5 ml. DMBA model of breast cancer is considered to be relevant for evaluation of the chemical's potential to cause human breast cancer development and is sensitive to hormonal manipulations (37, 85, 86). In the carcinogenesis studies, 20 mice per group are needed to provide sufficient power to detect statistically meaningful differences.

### Monitoring Mammary Tumors.

Four weeks after the final DMBA exposure, mice are checked for the presence of mammary tumors weekly. Once the first tumor is noted, the mice are checked twice per week. Tumor latency, incidence of mammary tumors per group, tumor multiplicity and the size and growth of the tumors (measured by a caliper) are recorded. Mice are sacrificed when detectable tumor burden approximates 10% of total body weight, as required by our institution. All surviving mice, including those which do not appear to develop mammary tumors, are sacrificed 20 weeks after carcinogen administration.

### Statistical Analysis Plan.

When assessing differences in mammary tumorigenesis, 20 mice per group are used. This provides sufficient statistical power to detect differences between different groups when the data are analyzed using Kaplan-Meier Survival Analyses with appropriate tests for significance, e.g., log-rank test. Parametric and non-parametric tests are used to determine tumor latency, multiplicity and growth. Differences in final tumor incidence among the groups are compared by utilizing Chi-square test.

### Example 4

Determine and compare changes in the methylation patterns of DNA obtained using buccal swaps in toddlers of mothers exposed to dietary intervention during pregnancy which increases maternal lignan levels, and of toddlers of control mothers.

### Description of the Cohort.

A study involves investigating pregnant women and their offspring participating in a duster-randomized controlled trial (RCT) to prevent gestational diabetes mellitus (GDM). Intervention included intensified counselling on diet and physical activity, integrated in routine maternal center visits. The protocol of the trial was shown feasible in a pilot study (87). Recruitment to the study was finished in December 2008, and the last women in the study gave birth in July 2009. This RCT includes women from 14 areas in Pirkanmaa, Finland; 410 women in the intervention and 590 women in the non-intervention group (total N=1000). The RCT included pregnant women with at least one of the following criteria: age equal to or higher than 40 years, body mass index at least 25 kg/m2, GDM in previous pregnancies or diabetic relative. Measurements included oral glucose tolerance tests, and measurements of weight, blood pressure, waist circumference, and lipid levels.

Women in the intervention group were advised to consume more fruits, vegetables and whole grains than the control group (87); this diet increases circulating lignan levels (manuscript submitted). Since lignans can induce epigenetic changes (88), this maternal dietary intervention could reverse the effects of high in utero E2 environment on the epigenome and reduce breast cancer risk.

### Biological Material Available.

Blood has been collected (twice) from the pregnant women during the first (weeks 8-12) and last trimesters (weeks 26-28); these are available for our study. Half of these women were subjected to dietary intervention. Phytoestrogen levels as well as the levels of E2 in some of these samples are first measured, and then four groups of women who were either in the dietary intervention or control groups and who had low or moderate phytoestrogen levels are selected (n=30 per group). DNA is collected from 10 daughters per group and is used for epigenetic assays to identify, in the daughters, epigenetic signatures related to higher in utero phytoestrogen levels that were know from our animal model studies increase later breast cancer risk.

### End-Points.

Changes in the levels of different endocrine disruptors, mainly plant derived phytochemicals and BPA, are determined in pregnant mothers in the control and dietary intervention groups. DNA collected from buccal swaps is assayed to determine whether the levels of endocrine disruptors in the blood of pregnant women and/or pregnancy dietary intervention affected methylation patterns in the DNA obtained from buccal swaps.

### Results.

The current risk assessment methods assume that the most susceptible rodent species (rats and mice) represents the most susceptible human. Therefore, an investigation can also performed to determine whether methylation changes in the DNA obtained by buccal swaps from toddlers whose mothers were consuming either an intervention diet containing high levels of fruits, vegetables or whole grain fiber or control diet, for the comparison of epigenetic markers in the mouse model vs. humans. This study can use an on-going cohort study in Finland, led by Dr. Riitta Luoto at the University of Tampere, Finland. In that study, the mothers in the intervention group exhibit higher levels of phytochemicals than the mothers in the control group. It can be investigated whether these changes are translated as differences in methylation patterns among the toddlers of the mothers in the intervention trial.

### Example 5

Enhanced tumorigenesis caused by in utero exposures to EDCs during embtryogenesis can be reversed by treating adult mice with epigenetic changes erasing drugs.

### Animals and Diets.

Time pregnant C57BL/6 female mice were obtained from Harlan (Harlan-Teclad, USA) on gestation day 6, and were randomly assigned into four groups on the following day (gestation day 7). The basic diet formulation in the study was a semi-purified AIN93G (the American Institute of Nutrition) modified to replace soy oil for corn oil; all the diets of the four treatment groups were purchased from Harlan (Harlan-Teclad, USA). All four of the diets were isocaloric, as ethinyl estradiol (EE2), bisphenol A (BPA), and genistein (GEN) were added in the diet at the expense of comstarch. Additions to the basic diet were as follows: 1) cornstarch [control]; 2) EE2 0.1 ppm; 3) BPA 0.155 ppm; 4) GEN 500 ppm. EE2 and BPA were purchased from Sigma Chemicals (St. Louis, Mo., USA), GEN was a gift from Dr. William Helferich (University of Illinois, Urbana, Ill., USA), and they were minimum 95% pure.

The pregnant dams were fed these diets until the day of the delivery of their pups (Post Natal Day, PND, 0). Starting PND 0, all mice were fed the control diet until the end of the study. Female (and some male) mice born from the dams fed with the different above mentioned diets were used in the subsequent studies. The study outline is shown in FIG. 12. Animals were housed in a temperature- and humidity-controlled room under a 12-hour light-dark cycle. All animal procedures were approved by the Georgetown University Animal Care and Use Committee, and the experiments were performed following the National Institutes of Health guidelines for the proper and humane use of animals in biomedical research.

### Analysis of Puberty Onset and Stage of Estrus Cycle of Female Mice.

To assess possible changes in sexual maturation due to in utero ECD exposures during embryogenesis, the day of puberty onset was recorded, as defined based on vaginal opening (VO) in female mice. The mice were examined once a day starting on PND 21. In sexually mature mice, the stage of estrus cycle was determined based on the cytology of their vaginal smear (sampled by pipetting 20 μl PBS in and out of vagina onto a slide) as judged under the microscope. Stage of estrus cycle was determined for each mouse before necropsy and sample collection.

### Blood and Tissue Sample Collection.

For ethical reasons non-pregnant littermate females were used for blood collection instead of the dams, and the samples were taken on the last day of their dietary exposure. Blood was collected by heart puncture of anesthetized mice prior to euthanasia, and serum was separated by centrifugation (1000×g for 10 min) and stored frozen until hormone measurements.

One of the two #4 abdominal mammary glands was dissected and fixed in Carnoy's fixative and processed into whole mounts as previously described (8). The other #4 abdominal gland was snap frozen in liquid nitrogen and stored in −80° C. for future analysis. One of the two thoracic #2-3 mammary glands was fixed in neutral buffered 10% formalin, embedded in paraffin and processed for histology using routine methods, and the other was snap frozen in liquid nitrogen, also stored in −80° C. for future analysis. Uterus was dissected, and one horn with the ovary and vagina attached was fixed in 10% formalin, embedded in paraffin and processed for histology, and the other horn and one ovary were frozen in liquid nitrogen and stored in −80° C.

### Analysis of Mammary Gland Morphology.

To assess possible changes in mammary gland morphology, whole amounts of the 4th abdominal glands obtained on PND 27 were processed as previously described (4, 72). The total number of terminal end buds (TEBs) was counted. Identification of TEBs was based on the guidelines established by Russo and Russo (64). The total area (in pixels) of epithelium of the peripubertal (PND 27) mammary glands was analyzed by using ImageJ software (available through NIH web site) on digitized pictures of mammary whole mounts with ducts manually intensified black against white background (shown as an insert in FIG. 16). Ductal elongation (mammary epithelial outgrowth) on PND 50 was measured as the growth in millimeters from the end of the lymph node to the end of the epithelial tree. All the analyses were done blindly using an Olympus dissecting microscope.

### Analysis of Mammary Tumorgenesis.

Mammary tumors were induced by administration of a subcutaneous injection of Medroxyprogesterone Acetate (MPA) to mice at 6 weeks of age, followed by administration of 1 mg 7,12-dimethylbenz(a)anthracene (DMBA) (Sigma, St Louis, Mo.) weekly for four weeks (weeks 7, 8, 9, 10) (23). The carcinogen was dissolved in corn oil and administered by oral gavage in a volume of ca. 0.1 ml. One week after the last DMBA, some of the mice were started on Hydralazine (5 mg/kg/day) and Valproic acid (1.16 g/kg/day) treatments. Animals were examined for mammary tumors by palpation once per week. Tumor growth was monitored for 16 weeks after the last dose of DMBA administration. All surviving animals were sacrificed at that point for sample collection. The end-points for tumor data analysis were (i) latency to tumor appearance, (ii) the number of animals with tumors (tumor incidence), and (iii) the cumulative tumor area (tumor burden). During the follow-up, animals in which tumor burden approximated 10% of total body weight were euthanized, as required by the ethical guidelines of our institution.

### Evaluation of in Utero Exposure's Effects on Growth and Sexual Maturation of the Pups, and Immediate Adverse Effects of E2, BPA and GEN on Adult Mice.

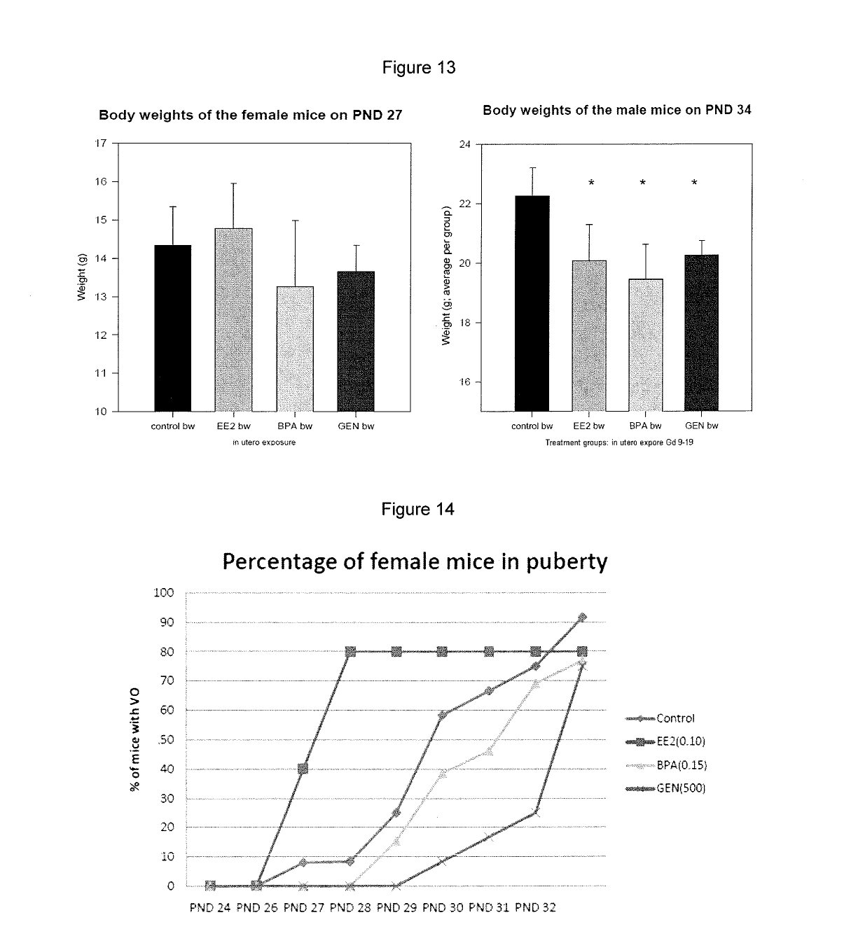

No statistically significant differences in body weights were observed in female pups on PND 27, a few days prior to their puberty onset (FIG. 13). In contrast, all the in utero exposes reduced male pups' body weights when measured (on PND 34) a few days after male pups' puberty onset, indicating an adverse effect of the in utero exposures (FIG. 13).

Puberty onset is considered an important parameter informing about possible adverse effects of in utero exposure, and in women an early onset of puberty has been shown to be a risk factor for breast cancer. Of the female pups only those exposed to EE2 had a significantly accelerated puberty onset, i.e. on PND 27 instead of PND 32 (FIG. 14); in all the other groups >75% of the female pups had entered puberty on PND 32.

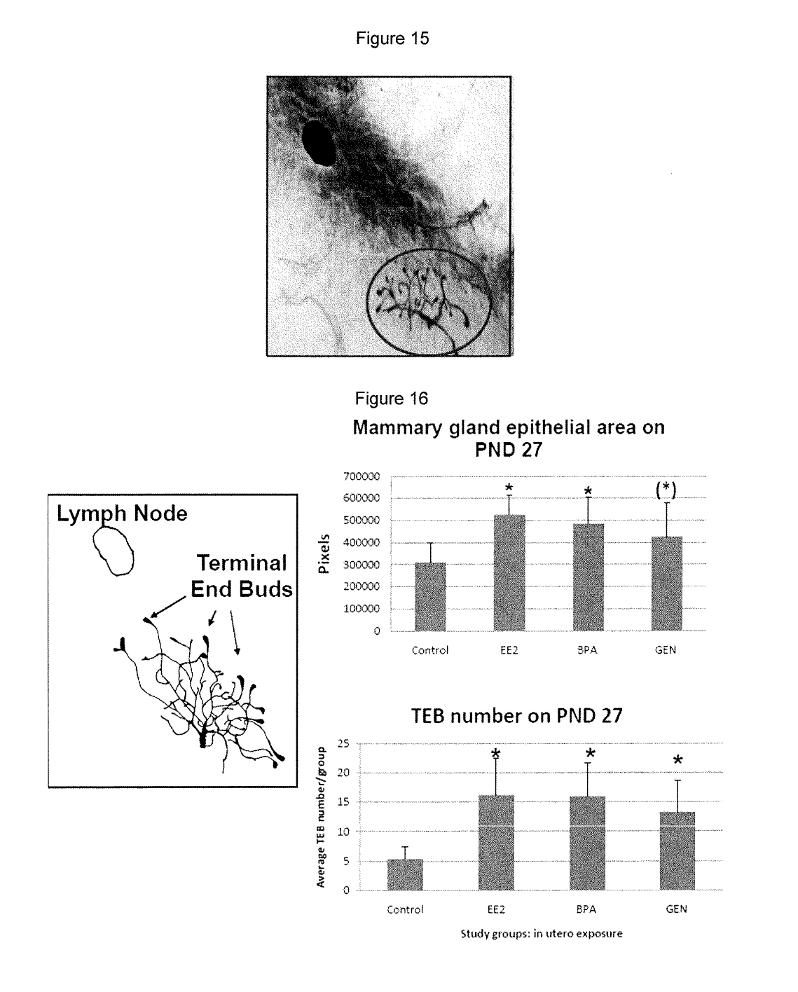

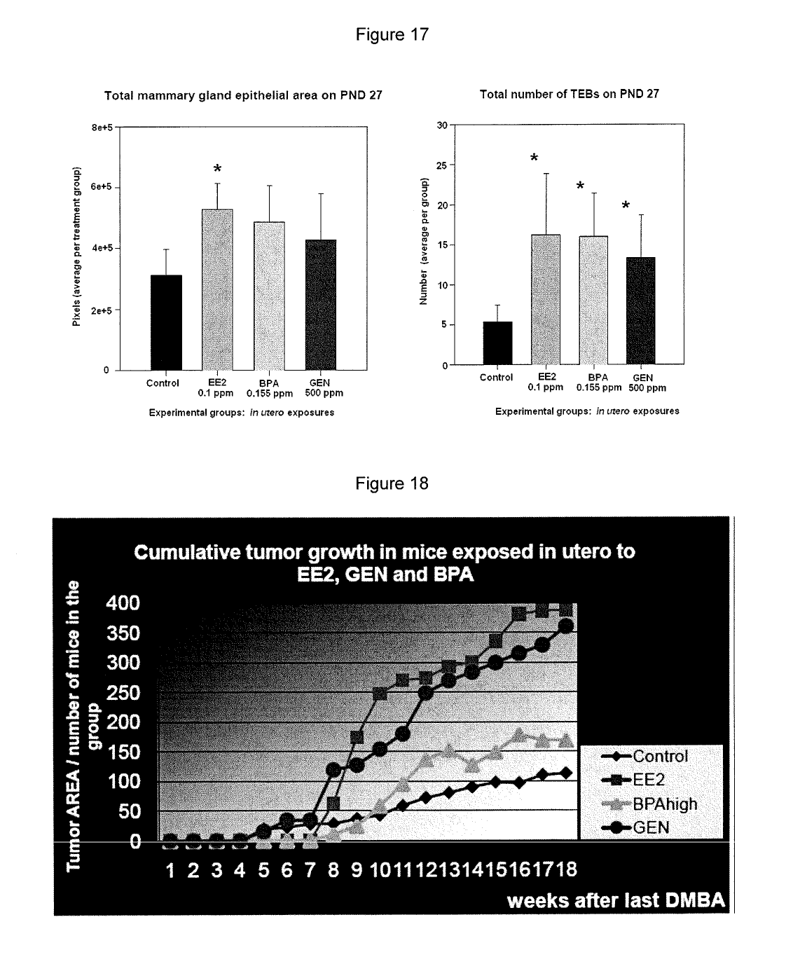

As the male pups showed a statistically significant decrease in their body weights due to the in utero exposures, possible adverse effects on male sex hormones were assessed. Mammary gland growth in males was analyzed using the mammary gland wholemount technique. Male mice lack nipples, and depending on the mouse strain they also completely lack mammary epithelium or can have a rudimentary epithelial tree at the time of birth, which fails to develop further as they age. The early mammary gland development is very sensitive to uterine environment. Typically, in male pups puberty starts some days later compared to females of the same strain and environment (onset was not individually assessed in this study). Therefore the male pups were analyzed at the age of 34 days at which time they had all entered puberty, as judged based on their balanopreputial separation (BPS). Most of the male pups had rudimentary mammary ductal structures (FIG. 15), but no additional development, such as elongation of ducts, was detected in any of the mice in any group, indicating that none of the exposures caused major adverse effects on mammary gland maturation of male mice.

In addition, possible immediate adverse effects of EE2, BPA and GEN exposure for adult mice were evaluated using the non-pregnant littermates of dams. Their body weights were recorded and the mammary gland morphology examined on the last day of their dietary exposure. No significant differences were observed between the groups in terms of body weight or mammary epithelial development (data not shown).

### Effects of in Utero Exposures on Mammary Gland Morphology.

Mammary gland morphology of female mice was assessed at PND 27 using wholemount technique to reveal possible changes due to the in utero ECD exposures. Mammary gland growth was clearly affected; all the in utero exposures significantly increased the number of TEBs (FIG. 16a), the growing points of the mammary epithelium likely to contain (most of the) stem cells in a pubertal mammary gland. This was reflected also in the total epithelial area of the glands (FIG. 16b), which was significantly increased in the EE2 and BPA exposed female mice, and nearly significantly increased also in the GEN exposed females.

### Enhanced Mammary Tumorigenesis Caused by in Utero Exposure to EE2, GEN and BPA.

Mammary tumorigenesis was studied using a model and treatment regimen generally used to induce mammary tumors in wild type mice (23), i.e. mice not prone to mammary tumorigenesis through e.g. genetic manipulation. Enhanced mammary tumorigenesis was observed in mice exposed in utero to EE2, GEN and BPA, both in terms of earlier onset of tumor growth and of cumulative tumor burden, compared to controls (FIG. 18). The results demonstrate that the cumulative tumor burden was approximately four times higher in the mice exposed to EE2 and GEN in utero, and 1.5 times higher in the mice exposed to BPA in utero compared to the group exposed to control diet in utero.

### Epigenetic Changes in the Mammary Gland Due to ECD Exposure During Embryonal Development Sensitize to Mammary Tumorgenesis in Adulthood.

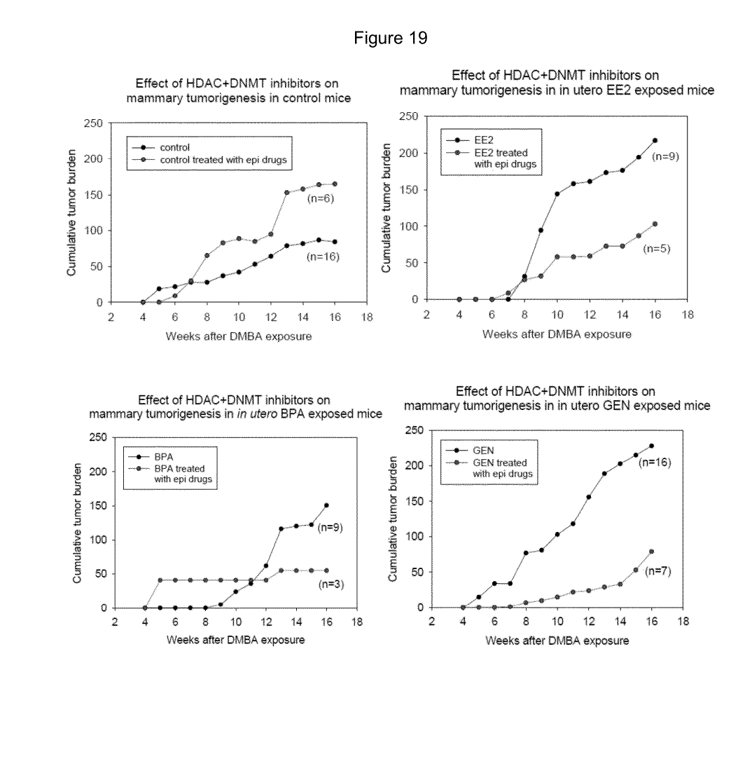

In order to investigate possible epigenetic changes involved in the mammary glands of female mice exposed to EDC in utero that could contribute to increased mammary tumorigenesis in adulthood, a proof of principle type of an experiment was designed and conducted. Tumor study mice were treated with drugs that inhibit DNA methylation (Hydralazine) and histone deacetylation (Valproic acid), starting one week after the last carcinogen administration and continuing daily through the end of the study. The results demonstrate clearly a complete reversal of mammary tumorigenesis in the in utero ECD exposed mice, whereas no change was detected in the control mice (FIG. 19). Taken together these results show that the increased susceptibility to breast cancer due to in utero exposures to EE2, GEN and BPA is likely to be mediated by epigenetic changes taking place in early (embryonal) mammary gland development and persisting throughout development to adult tissue.

### Permanent Changes in the Expression of Dnmt1 in the Mammary Glands Exposed to ECD During Embryogenesis.

The expression levels of Dnmt1 was determined in the mammary glands of adult animals exposed to ECD in utero. Dnmt1 is the gene that encodes the enzyme DNAse methylransferase 1, known to be involved and crucial in methylating DNA. Mammary glands of animals from a similar study were used, and we observed a persistent and significant increase in the expression of Dnmt1 expression in the mammary glands of adult animals exposed to ECD during embryogenesis (FIG. 6). Mammary glands of the in utero exposed mice at different ages are analyzed.

### Example 6

### Maternal Diet During Pregnancy Increases Mammary Cancer in Three Generations of Offspring

The risk of many chronic adult-onset diseases is influenced by exposures during early development, and in some cases the risk is transmitted beyond a single generation (100). Using rodent models, its has been shown (4, 7) that in utero estrogenic exposures increase the risk of mammary cancer in the adult female offspring. This study investigated whether exposing pregnant rats (F0) to a high fat (HF) or ethinyl estradiol (EE2) supplemented diet would affect mammary cancer risk not only in daughters (F1) but also in granddaughters (F2) and great-granddaughters (F3). Mammary tumorigenesis was higher in daughters and granddaughters of HF rat dams and in daughters, granddaughters and great-granddaughters of EE2 rat dams. Outcross experiments revealed that increased mammary cancer risk can be transmitted to granddaughters through both in utero HF exposed F1 females or males, but is only transmitted through in utero EE2 exposed F1 females. The increase in mammary cancer risk in any given HF or EE2 generation correlated with low levels of mammary gland differentiation and increased Dnmt1 expression in adulthood. Our study shows the importance of early life exposures on mammary cancer risk, and that this risk persists for up to three generations (F1-F3) without any further intervening exposure. Thus, breast cancer risk that is affected by maternal nutritional exposures during pregnancy could be inherited through non-genetic means. These observations, if confirmed in humans, can lead to new and more effective approaches to help reduce the incidence of breast cancer.

Family history is a significant risk factor for breast cancer (101). However, genetic mutations in high penetrance genes, such as BRCA1 and BRCA2, account only for a small proportion of familial breast cancers (102, 103). Thus, it is possible that many familial breast cancers may not be transmitted through inheritance of gene mutations but mediated through other mechanisms, including heritable epigenetic changes caused by in utero exposures.

Maternal diet during pregnancy can have long lasting effects on an offspring's health (104). It has been shown (4, 105) that high fat (HF) intake during pregnancy increases mammary cancer risk in animal models, perhaps through HF diet-induced increase in pregnancy estradiol (E2) levels (4).

Exposing pregnant rats to E2 has similar effects on the offspring mammary cancer risk (4); either prenatal or perinatal exposure to the synthetic estrogen diethylstilbestrol (DES) also increases mammary cancer risk in rats (7, 106) and mice (107) and breast cancer risk in humans (38, 108, 109). More recent evidence suggests that some disease traits resulting from in utero exposures can be transmitted epigenetically through multiple generations (110-112). Exposure of the developing male fetus to endocrine disruptors reduces fertility and causes prostate and kidney abnormalities that can persist for four consecutive generations (110-112).

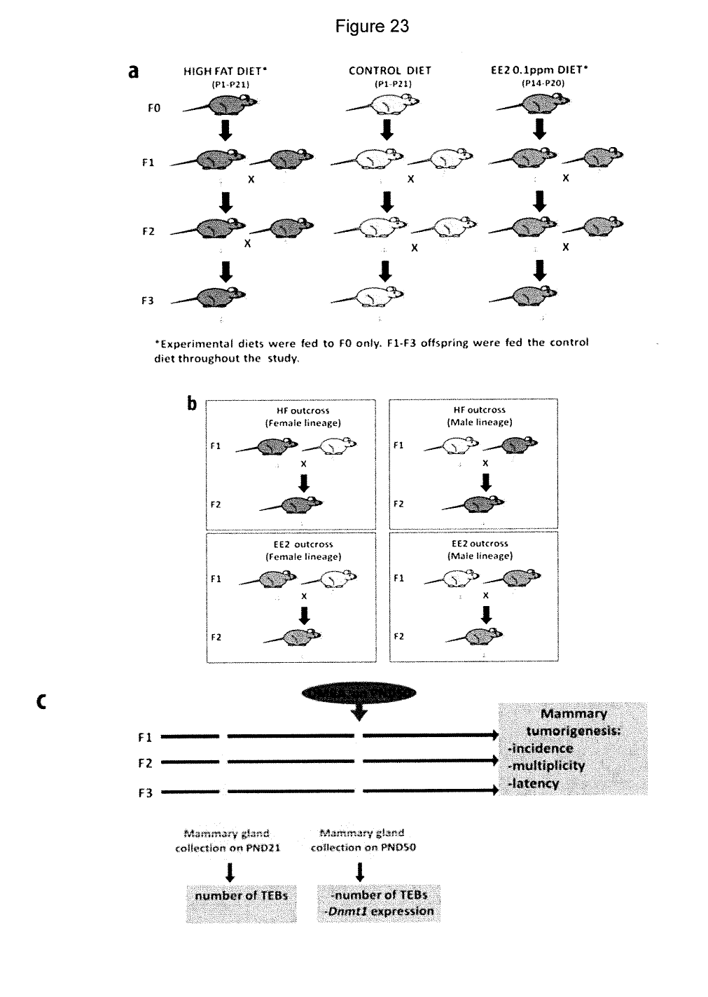

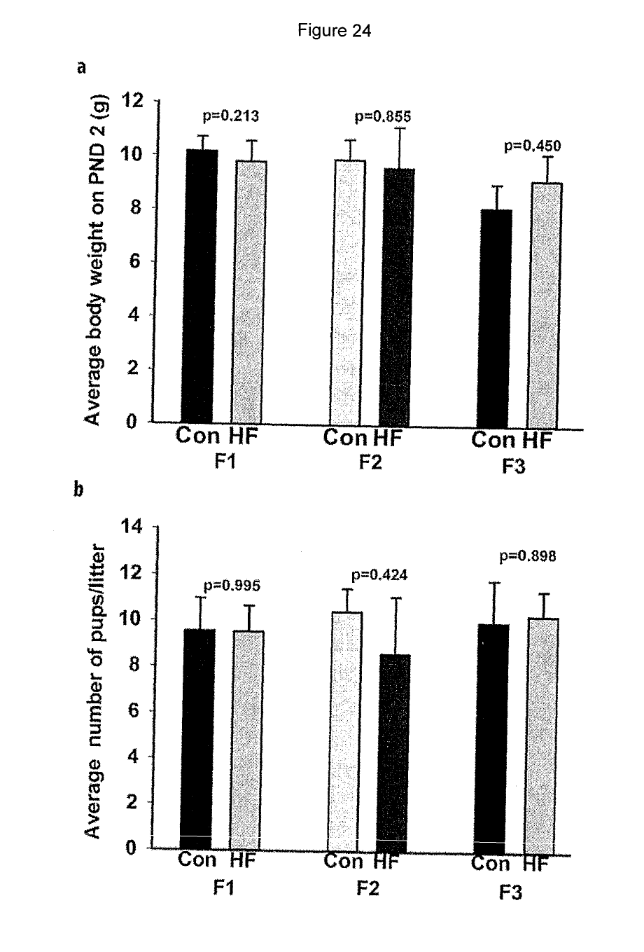

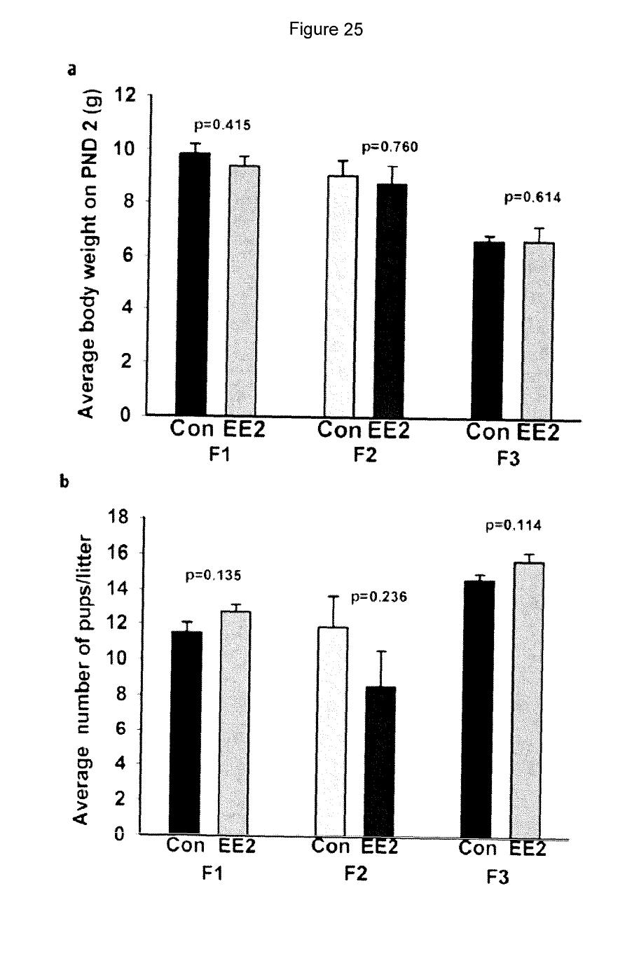

This study examined whether in utero exposures to HF diet or synthetic E2 lead to multigenerational inheritance of mammary cancer in a carcinogen DMBA-induced breast cancer model. Pregnant Sprague-Dawley rats (F0) were fed AIN93G control diet or an isocaloric AIN93G based HF diet, containing 43% energy from corn oil, throughout gestation. Another group of pregnant rats was fed AIN93G diet supplemented with 0.1 ppm ethinyl estradiol (EE2) from day 14 to day 20 of pregnancy. F1 females were mated with F1 males from the same group to produce the F2 generation. The F3 generation was produced in the same manner (FIG. 23). No sibling mating was carried out. The F1, F2 and F3 offspring were maintained on the AIN93G control diet for the duration of the experiment HF or EE2 diet of the F0 dams did not affect litter size or pups weight in any of the three generations studied (FIGS. 24 and 25).

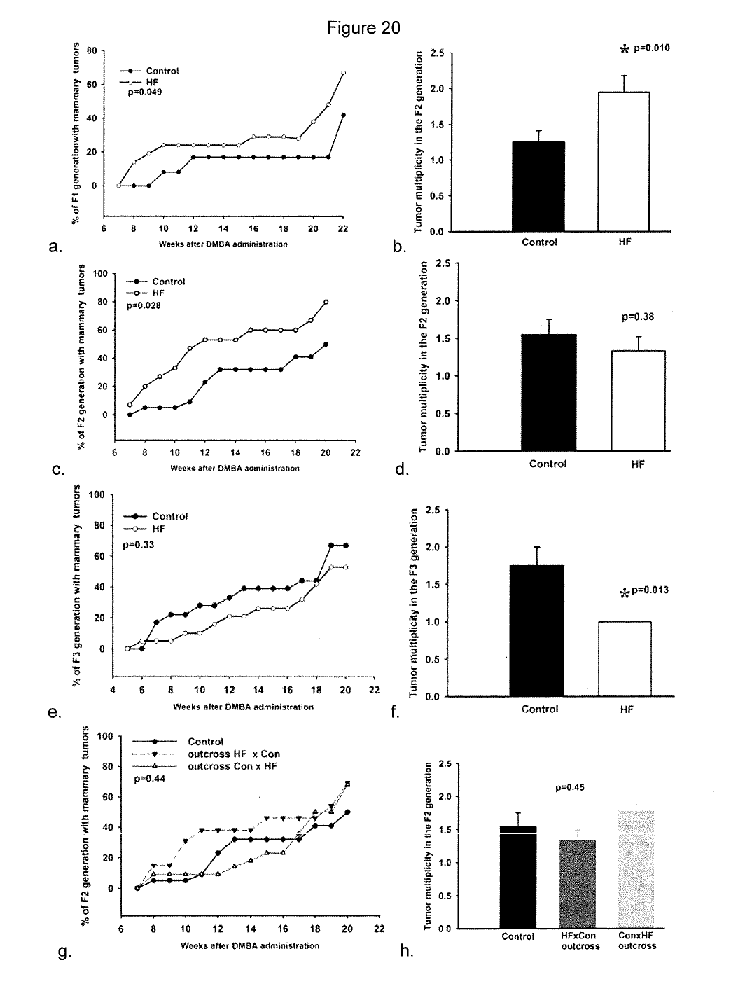

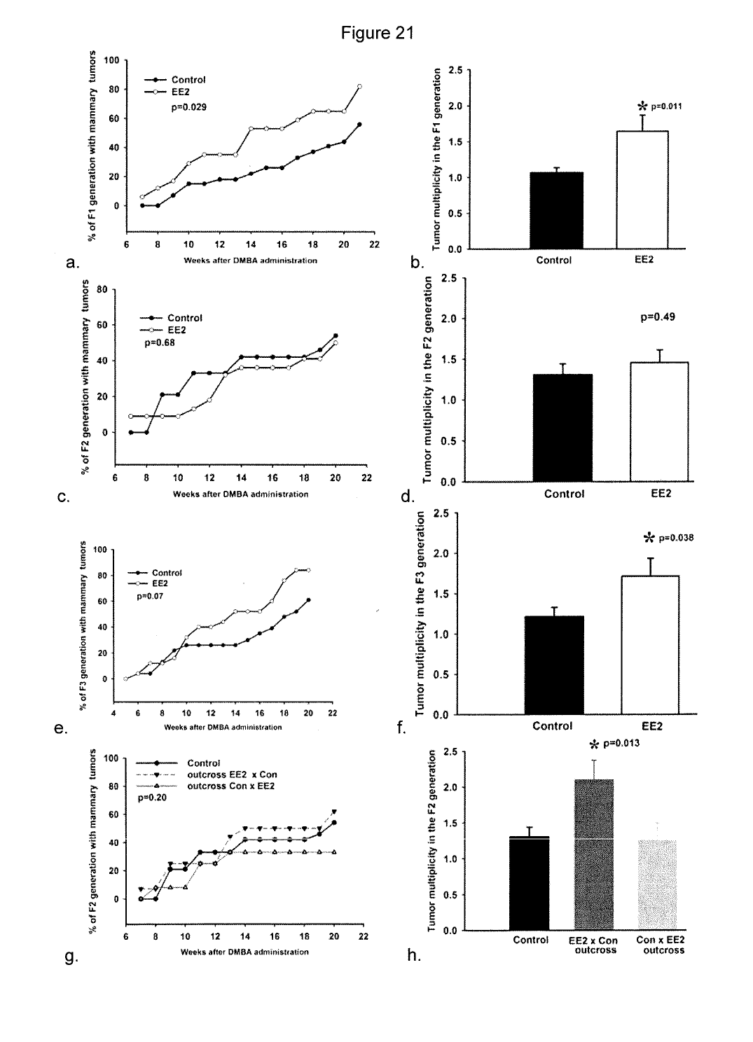

Daughters (F1 generation) of rats dams exposed to HF or EE2 during pregnancy had higher mammary tumor incidence and multiplicity (FIGS. 20A and 20B; FIGS. 21A and 21B) compared with controls (HF: p=0.029 and p=0.010; EE2: p=0.029 and p=0.011) as reported before (4).

Effects of HF or EE2 exposure on pregnant F0 dams on mammary cancer risk in the F2 and F3 generations were then examined. In the HF granddaughters (F2 generation) mammary tumor incidence (p=0.028), but not multiplicity (p=0.38), was higher compared to the control group (FIGS. 20C and 20D). Mammary tumor incidence did not differ (p=0.33) between the control and HF great-granddaughters (F3 generation), however, tumor multiplicity was lower (p=0.013) in the HF offspring (FIGS. 20E and 20F). In the EE2 granddaughters (F2 generation), neither tumor incidence (p=0.68) nor multiplicity (p=0.49) was statistically different from the controls (FIGS. 21C and 21D). However, EE2 great-granddaughters (F3 generation) had significantly higher tumor multiplicity (p=0.038) compared to controls. Mammary tumor incidence was also higher, but not statistically significant (p=0.07) in the EE2 F3 generation (FIGS. 21E and 21F) compared to controls.

Histopathologic analysis indicated that the majority of mammary tumors across all three groups were malignant (adenocarcinomas or solid carcinomas).

Both HF and EE2 in utero exposures have been shown to alter mammary gland morphology (4), mainly by increasing the number of terminal end buds (TEBs). These undifferentiated mammary structures are the sites of malignant transformation in rat mammary gland (113), and human breast (113).

The information in Tables 2 and 3 is used to examine whether these effects persisted in the F2 and F3 generation offspring. On postnatal day (PND) 21, the number of TEBs was increased in the mammary glands of both HF and EE2 daughters (F1) (p=0.032 and p=0.033) and granddaughters (F2) (p=0.004 and p=0.006). However, in great-granddaughters (F3), increase in the number of TEBs was only seen in the offspring of EE2 exposed dams (p=0.014). On PND50, the number of TEBs was increased in mammary glands of daughters (F1) (p=0.039) and granddaughters (F2) (p=0.044), but not great-granddaughters (F3), of HF exposed dams. In the EE2 offspring, however, there were no significance differences in the number of TEBs on PND 50 in any generation.

To investigate whether mammary cancer risk could be transmitted either through the female or male lineages, F1 unexposed males were mated to F1 exposed females or F1 unexposed females were mated to F1 unexposed males (FIG. 23B). Mammary tumor incidence was higher in both HF F2 outcross groups (HF♀-C♂:68%; and C♀-HF♂: 69%) compared to controls (50%), but did not reach statistical significance (p=0.44) (FIG. 20G). These results, however, suggest that the effects of HF in utero exposure on mammary cancer risk can be transmitted through both the female or male germline. EE2 outcross experiments show that only 33% of offspring resulting from C♀-EE2♂ breeding developed mammary tumors, whilst 62% of the EE2♀-C♂ offspring developed mammary tumors (FIG. 21G). Levels of tumor multiplicity were significantly higher in the EE2♀×C♂ outcross group compared to controls (p=0.013) (FIG. 21H). Thus, unlike HF, EE2 exposure had an opposite effect on developing male and female embryos regarding their ability to transmit susceptibility to mammary cancer to their offspring.

Changes in mammary gland morphology in the outcross offspring were assessed. (Tables 2 and 3). On PND21, mammary glands of Con♀-HF♂ outcross (p=0.005), but not HF♀-Con♂ (p=0.654) had a higher number of TEBs than the controls. Mammary glands of the EE2♀-Con♂ outcross also had a significantly higher number of TEBs (p=0.005) on PND21, but not on PND50 (p=0110). Lower number of TEBs was seen on PND50 in the Con♀-EE2♂ group, compared to the controls (p=0.003).

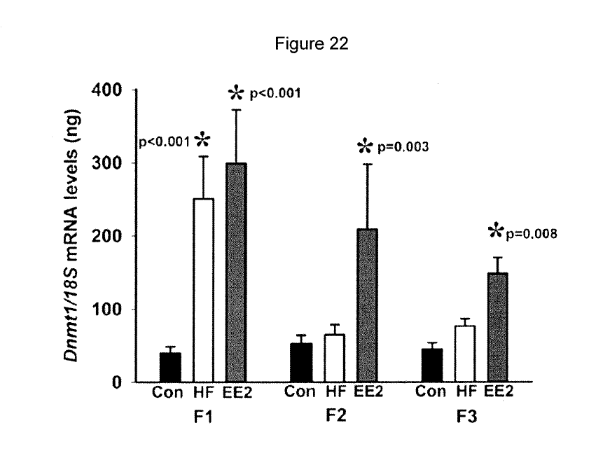

Transgenerational inheritance of disease can be mediated by alterations in DNA methylation (100, 114). After DNA replication, DNMT1methylates the CpG sites on the daughter DNA strand to maintain the parental pattern of methylation (115, 116), and is also involved in de novo methylation (117, 118). Maternal exposure to protein restricted diet during pregnancy reduces Dnmt1 mRNA expression and hepatic glucocorticoid receptor methylation (119). HF and EE2 exposures during pregnancy were analyzed to determine whether such exposures affected mRNA expression of Dnmt1 in the mammary glands of F1-F3 generations on PND50 (FIG. 22). Both HF and EE2 in utero exposures increased the levels of Dnmt1 mRNA in F1 generation mammary gland (p<0.001 for HF and EE2), and this increase persisted in EE2 to F2 (p=0.003) and F3 (p=0.008) generation offspring but not in the HF offspring (FIG. 22). Hence, an inheritable epigenetic trait, likely maintained by high Dnmt1 levels, can partly explain the reduced mammary gland differentiation and the increased mammary cancer risk observed in the offspring at least following EE2 exposure.

Interestingly, it was observed that maternal HF increases mammary cancer risk in the F1 and F2 offspring, while maternal EE2 exposure increases the risk in three consecutive generations (F1-F3), suggesting that either EE2 is a more potent exposure or that two different mechanisms of multigenerational transmission exist Outcross experiment data showed that transgenerational differences in mammary tumorigenesis depend on whether the mother or father had been exposed in utero to EE2. The lack of difference between the EE2 and control groups in the F2 geneneration suggests that reduced mammary tumor incidence in the offspring of F1 EE2 males counteracts the increase observed in the offspring of F1 EE2 females. Increase in mammary tumorigenesis following in utero HF exposure, however, was transmitted equally through the female or male germline. Differences in inheritance through male and female germlines in the EE2 group are also likely to reflect the fact that females already have all their germ cells during their fetal development, whereas in males these cells are only produced after puberty onset (120). Two recent studies show that transmission of DNA methylation occurs mainly through maternal gametes (121, 122). Thus, in utero exposures could affect the methylation patterns of the female embryo germ cell genome. In males, these exposures first affect primordial germ cells or other undifferentiated stem cells that will give rise to germ cells, starting at puberty. In this study, EE2 exposure can involve epigenetic transmission through female germ cells, whereas HF exposure can primarily affect stem cells that will give rise to germ cells in the next generation.

The differences in mammary tumorigenesis outcome regarding the HF and EE2-supplemented diets can also be due to different durations of the in utero exposures. The HF diet was fed to pregnant dams throughout pregnancy while the EE2 supplemented diet was fed from day 13 to 20 of pregnancy, since an earlier EE2 exposure tends to disrupt pregnancy. It is possible that in order for breast cancer risk to be transmitted through both the female and male germlines, the exposures need to occur within a certain window of development, as shown for other diseases (110).

This study demonstrates, for the first time, that maternal dietary exposure to EE2 and HF during pregnancy can initiate an inheritable increase in the offspring's breast cancer risk, persists up to three consecutive generations. If confirmed in humans, our findings represent a novel perspective on how breast cancer risk is transmitted across generations and could have marked implications for breast cancer prevention.

### Animal Studies.

Pregnant rat dams (n=10-12/group) were fed one of the following diets:

AIN93G control diet (control, 17% energy from corn oil), isocaloric high fat diet (HF, 43% energy from corn oil) or ethinyl estradiol-supplemented AIN93G diet (EE2, 0.1 ppm). The HF diet was fed for dams through the duration of pregnancy (P) (21 days), while dams were fed EE2 supplemented diet between days P14-20. Once pups (F1) were born, all dams received the control diet. F1 and F2 females (n=8-10) were mated on PND 60 with males from the same group to produce the F2 and F3 generations. All F1 and F2 pregnant dams were fed control diet for the extent of pregnancy. Outcross experiments were also performed: F1 females or males exposed to EE2 or HF diet in utero were crossed with control males or females. All animal procedures were approved by the Georgetown University Animal Care and Use Committee.

### Mammary Tumorigenesis.

Mammary tumors were induced by exposing F1-F3 female offspring (n=12-271 group) to 10 mg 7,12-dimethylbenz[a]anthracene (DMBA) on PND 50 (123). Tumorigenesis (incidence and multiplicity) was monitored for 20 weeks.

### Mammary Gland Morphology.

Mammary gland whole-mounts (n=5-6/group), obtained on PND21 and 50, were processed as described (100), and the number of terminal end buds (TEB) was counted under a light microscope by two independent investigators.

### Quantitative Real-Time (RT) PCR.

Total mammary RNA (n=3-6/group) was used as a template for random primed cDNA synthesis (TaqMan MultiScribe Reverse Transcriptase and RT-PCR Reagents, AppliedBiosystems, Roche, N.J., USA). cDNA samples were then used as templates for quantitative RT PCR analysis with previously described specific primers for the target gene, rat Dnmt1 (124) using QuantiTect SYBR green PCR kit (Qiagen Inc., Valencia, Calif.). Each sample was normalized to the reference gene 18S rRNA (27).

Mammary glands were dissected on PND21 (n=5-6/group) and PND50 (n=5-6/group) in F1-F3 generation offspring, stained with Carmine Alum solution and examined under a microscope.

The results are shown in Table 2. All values are expressed as the mean±s.e.m. *P<0.05, versus control for a given generation: t-test or Mann-Whitney Rank Sum test and one-way ANOVA followed by Holm-Sidak multicomparison procedure (F2 outcross groups).

TABLE 2
Transgenerational effects of maternal HF diet during
pregnancy on mammary gland morphology (number of
TEBs) in Sprague-Dawley rats on PND21 and PND50

| | Number of TEBs | | Number of TEBs | |
|---|---|---|---|---|
| Group | PND 21 | P value | PND 50 | P value |
| | F₁ generation | | F₁ generation | |
| Control | 18.0 ± 1.3 | | 25.4 ± 1.8 | |
| High Fat | 22.8 ± 1.4* | 0.032 | 32.0 ± 2.0* | 0.039 |
| | F₂ generation | | F₂ generation | |
| Control | 20.5 ± 1.3 | | 23.0 ± 4.3 | |
| High Fat | 33.4 ± 3.0* | 0.004 | 36.4 ± 3.5* | 0.044 |
| | F₃ generation | | F₃ generation | |
| Control | 29.8 ± 2.2 | | 28.8 ± 2.6 | |
| High Fat | 25.6 ± 5.1 | 0.473 | 24.4 ± 1.6 | 0.201 |
| | Outcross | | Outcross | |
| | (F₂ generation) | | (F₂ generation) | |
| Con [symbol] x HF | 34.8 ± 3.5* | 0.005 | 32.0 ± 3.6 | 0.199 |
| outcross | | | | |
| HF [symbol] x Con | 25.5 ± 3.8 | 0.654 | 23.8 ± 2.5 | 0.199 |
| outcross | | | | |

[symbol]

indicates data missing or illegible when filed

Mammary glands were dissected on PND21 (n=5-6/group) and PND50 (n=5-6/group) in F1-F3 generation offspring, stained with Carmine Alum solution and examined under a microscope.

The results are shown in Table 3. All values are expressed as the mean±s.e.m. *P<0.05, versus control for a given generation: t-test or Mann-Whitney Rank Sum test and one-way ANOVA followed by Holm-Sidak multicomparison procedure (F2 outcross groups).

TABLE 3
Transgenerational effects of maternal EE2 diet during
pregnancy on mammary gland morphology (number of TEBs)
in Sprague-Dawley rats on PND21 and PND50

| | Number of TEBs | | Number of TEBs | |
|---|---|---|---|---|
| Group | PND 21 | P value | PND 50 | P value |
| | F₁ generation | | F₁ generation | |
| Control | 21.2 ± 0.9 | | 25.8 ± 3.2 | |
| EE2 | 25.3 ± 1.3* | 0.033 | 32.5 ± 2.3 | 0.121 |
| | F₂ generation | | F₂ generation | |
| Control | 20.2 ± 2.2 | | 19.0 ± 2.0 | |
| EE2 | 30.8 ± 1.9* | 0.006 | 22.2 ± 3.8 | 0.474 |
| | F₃ generation | | F₃ generation | |
| Control | 21.3 ± 3.0 | | 23.8 ± 3.3 | |
| EE2 | 33.0 ± 2.1* | 0.014 | 28.7 ± 3.4 | 0.337 |
| | Outcross | | Outcross | |
| | (F2 generation) | | (F2 generation) | |
| Con [symbol] x EE2 | 19.4 ± 1.5 | 0.997 | 11.0 ± 1.2* | 0.003 |
| outcross | | | | |
| EE2 [symbol] x Con | 32.7 ± 3.6* | 0.005 | 22.8 ± 1.6 | 0.110 |
| outcross | | | | |

[symbol]

indicates data missing or illegible when filed

Maternal diet during pregnancy increases mammary cancer in three generations of offspring where only the original pregnant dams (F0) carrying the F1 generation received experimental dietary exposures.

### Animals and Breeding:

Sprague-Dawley rats were used in all experiments. Animals were housed in a temperature- and humidity-controlled room under a 12-hour light-dark cycle. All animal procedures were approved by the Georgetown University Animal Care and Use Committee, and the experiments were performed following the National Institutes of Health guidelines for the proper and humane use of animals in biomedical research.

The F1, F2 and F3 generations were obtained as described below. Only the original pregnant dams (F0) carrying the F1 generation received experimental dietary exposures.

### F1 Generation:

Two females and 1 male per cage were mated. Treatment groups are described below.

### In Utero High Fat (HF) Exposure:

Pregnant rat dams (n=10-12) were divided into two groups: AIN93G control diet (17% energy from fat), and high fat diet (HF, 43% energy from fat). Both groups were fed the experimental diets for the extension of the pregnancy. The main source of fat in the high fat diet was corn oil (n-6 PUFA). This diet has been found to elevate pregnancy estradiol levels and mammary cancer in the offspring.

### In Utero Ethynyl-Estradlol (EE2) Exposure:

Pregnant rat dams (n=10-12) were divided into two groups: AIN93G control diet (17% energy from fat), and ethynyl-estradiol-supplemented diet (EE2, 0.1 ppm). The composition of the EE2-supplemented diet was similar to the control diet, except for the addition of 0.1 ppm of EE2. The control group was the control diet for the extension of the pregnancy, while the EE2 group was fed the control diet from day 1-13 and the EE2-supplemented diet from day 14-20 of gestation. One day before they delivered, all rat dams were switched to the control AIN93G diet.

The difference in the length of HF versus EE2 exposure was due to the fact that the high fat diet does not immediately increase E2 levels, however, high levels of EE2 added to the diet at the beginning of pregnancy can cause miscarriage of the fetuses. Further, pregnant dams have to be taken off from the EE2 diet one day before labor, because EE2 can impair maternal behavior.

Pregnant dams were weighed once a week to monitor changes in weight gain. The birth weight of pups and number of pups per litter were recorded. To avoid litter-effect, pups were crossfostered 1-2 days after dams gave birth. Pups from 2-4 dams were pooled and housed in a litter of 8-10 pups per nursing dam (which had the same dietary/hormonal exposure during pregnancy as the pups' mothers). All pups were weaned on postnatal day 21.

The F1 female offspring were then be used to breed subsequent generations, study mammary tumorigenesis and for mammary gland morphology.

### F2 Generation:

F1 exposed (EE2 or HF diet) female rats were mated with F1 exposed males. The control groups (AIN93G control diet) were mated in the same manner. Pregnant dams were fed a standard AIN93G diet throughout pregnancy. No sibling mating was carried out. Monitoring of pregnancy, pups cross-fostering and weaning was carried-out as described for the F1 generation.

Outcross experiments were also performed to determine whether the transgenerational effect on mammary cancer risk is transmitted to the female or male germ line or both. F1 females exposed to EE2 (EE29) or HF (HF♀) diet in utero were crossed with untreated control males (C♂). The reverse outcross was also performed: F1 males exposed to EE2 (EE2♂) or HF (HF♂) diet in utero were crossed with untreated control females (C♀).

### F3 Generation:

F2 females and F2 males from each exposure group were mated in the same manner as described above to produce the F3 generation. Pregnant dams were fed a standard AIN93G diet throughout pregnancy.

### Mammary Gland Harvesting:

On postnatal day (PND) 21 and 50, mammary glands of F1, F2 and F3 generation female pups (n=6 per group and age) were collected and used to study mammary gland morphology as described below.

### Mammary Gland Morphology:

The 4th abdominal mammary glands obtained from 21 and 50-day old F1-F3 offspring were dissected, stretched onto a slide, placed in a fixative solution and stained with a carmine aluminum solution to prepare whole mounts. Whole mounts (n=5-6 per group and age) were examined under the microscope and evaluated as described before (28) to assess the number of terminal end buds (TEBs).

Results were analyzed by t-test (or Mann-Whitney Rank Sum test) or one-ANOVA (when comparing outcross groups in F2 generation). Where appropriate, between groups comparisons after ANOVA were done by Holm-Sidak multi-comparison procedure.

### Mammary Tumorigenesis:

Mammary tumors were induced in 50-day-old (t 2 days) F1, F2 and F3 generation females (n=12-27/group) by administration by oral gavage of 10 mg of 9,12-dimethylbenz[a]anthracene (DMBA) (Sigma, St. Louis, Mo.) in 1 ml of peanut oil. Rats were examined for mammary tumors by palpation once per week, starting on week 3 post DMBA and continued for 20 weeks post DMBA. Tumor growth was measured using a caliper and the length, width, and height of each tumor were recorded. The end-points for data analysis were (i) latency to tumor appearance, (ii) the number of animals with tumors (tumor incidence), and (iii) the number of tumors per animal (tumor multiplicity), During the follow-up, those animals in which tumor burden approximated 10% of total body weight were sacrificed, as required by our institution. Differences in tumor latency and multiplicity were tested by t-test (or Mann-Whitney Rank Sum test) or one-way ANOVA (where appropriate, between groups comparisons after ANOVA were done using Dunn's multi-comparison procedure). Kaplan-Meier survival curves were used to compare differences in tumor incidence, followed by the log-rank test Tumor histopathology was analyzed.

cDNA Synthesis and Quantitative Real-Time PCR Analysis:

Two hundred ng of total RNA per sample (n=3-6) was used as a template for random primed cDNA synthesis with a recombinant Moloney murine leukemia virus reverse transcriptase (TaqMan MultiScribe Reverse Transcriptase and RT-PCR Reagents, AppliedBiosystems, Roche, N.J., USA), according to manufacturer's instructions. An RT enzyme-minus control reaction was also included. The cDNA samples were then used as templates for quantitative real time PCR analysis with previously described specific primers for the target gene, rat Dnmt1(29) using QuantiTect SYBR green PCR kit (Qiagen Inc., Valencia, Calif.) and an ABI Prism 7900 Sequence Detection System.

Each sample was run in triplicate, and the qPCR run was repeated twice. Absolute gene expression levels were determined using Applied Biosystems' SDS2.3 software and the standard curve method. Concentration of each sample was normalized to the reference gene 18S rRNA (30), and averages of the three runs are shown. Differences in Dnmt1 mRNA expression levels were tested by two-way ANOVA (after log-transformation), followed by Holm-Sidak multicomparison procedure.

### Example 7

### Estrogens Increase Breast Cancer Risk, Especially if the Exposure Occurs in Utero

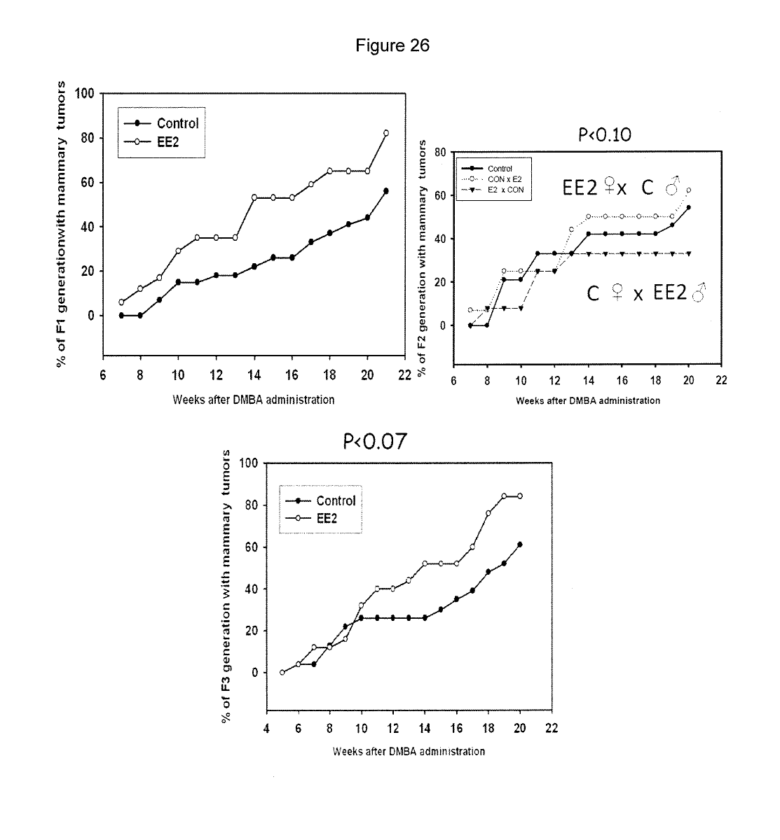

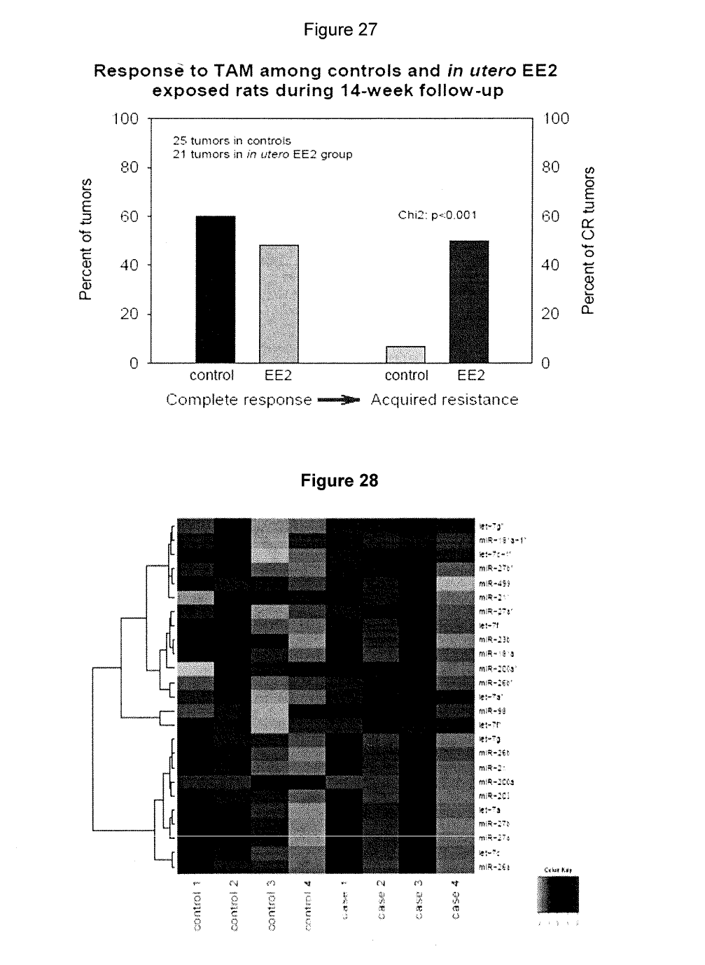

While breast cancer is the most common cancer in women, affecting 1 of 8 women in the US during their life-time, it is not known what causes this disease, how to identify those women who will develop breast cancer, nor how to reduce breast cancer risk. High life-time exposure to estrogens increases breast cancer risk, but sensitivity to estrogens varies during different periods of life. Findings in humans suggest that women exposed to an elevated estrogenic environment in utero through their pregnant mother experience an increased breast cancer risk. Animal studies show that exposing pregnant dams to either estradiol (E2), synthetic ethinyl estradiol (EE2) (4), or to a high fat diet that elevates pregnancy E2 levels increases mammary tumorigenesis among their female offspring. This increase is not limited to daughters, but occurs also in granddaughters and great granddaughters (FIG. 26). Further, in utero estrogenic exposures increase the risk of acquired tamoxifen (TAM) resistance from 7% in the control rats to 50% in the in utero E2 group (FIG. 27), indicating that excessive estrogenicity early in life affects not only the risk of developing breast cancer in multiple generations, but also response to antiestrogen (AE) treatment.

### Mechanisms Mediating the Transgenerational Effects of Maternal Estrogenic Exposures on Offspring's Breast Cancer Risk.

Recent evidence shows that some disease traits resulting from in utero exposures are transmitted epigenetically by changes in DNA methylation through multiple generations. Our study suggests some familial breast cancers can also be inherited epigenetically. Many women seem to have inherited susceptibility to breast cancer, yet less than 25% of familial breast cancers can be linked to a specific genetic mutation, such as germline mutations in tumor suppressor genes BRCA1 or BRCA2.

DNA methylation patterns are established during embryonic period, when methylation and non-coding miRNAs play an important role in normal development. DNA methylation has been identified as a key process encoding the consequences of gene-environment interactions during early development; these changes produce the observed phenotype(s). Expression of miRNAs, each of which can suppress as many as 200 posttranscriptional target genes, is regulated by methylation. We have found that maternal exposure to EE2 or a high fat diet leads to a permanent down-regulation of several miRNAs in the mammary gland. These same miRNAs also are down-regulated in MCF-7 breast cancer cells by E2 exposure (FIG. 28, only results in the EE2 offspring are shown).

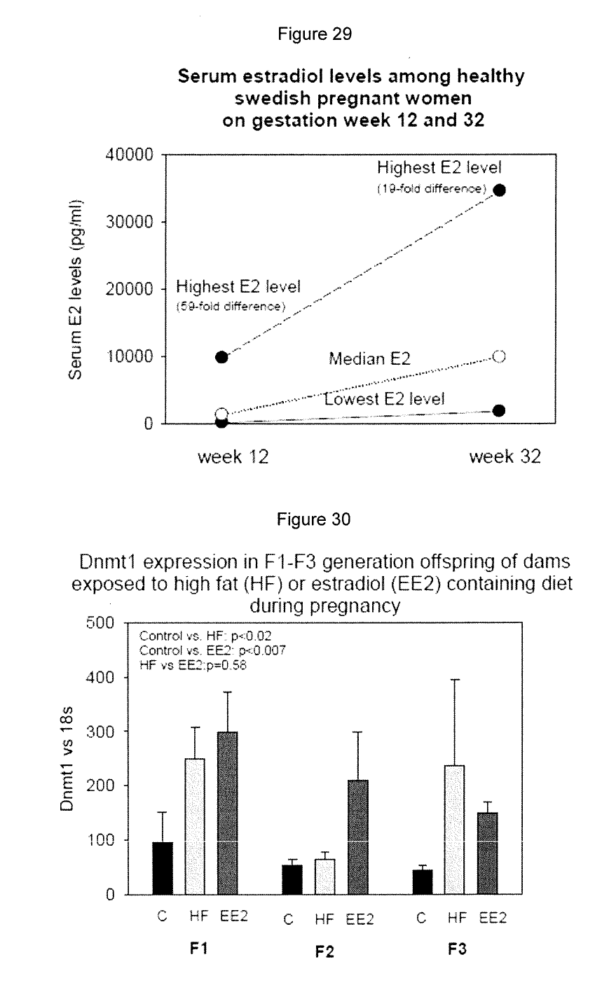

It is not clear what percentage of women have been exposed to an elevated in utero estrogenic environment, but it may be relatively high. In our study involving 286 healthy pregnant women, the inter-individual difference in E2 levels during gestation week 12 was 59-fold and during week 32 19-fold (FIG. 29). Further, approximately 22% of pregnant women had levels that were 1.5-fold higher than the mean pregnancy estrogen levels. In addition to the natural variability in pregnancy estrogen levels, humans are exposed daily to varying levels of endocrine disrupting chemicals (EDCs), some of which have known estrogenic activities. The best characterized in utero EDC exposure occurred between 1940s and 70s, when an estimated 5-10 million women in the US used synthetic estrogen diethylstilbestrol (DES) during pregnancy. “DES daughters” are now approaching the age when breast cancer is diagnosed, and findings generated thus far show that DES daughters have an increased risk to develop breast cancer. Among the EDCs humans are currently being exposed to is Bisphenol A (BPA); over 90% of people are estimated to have detectable levels of BPA in their system (24), including pregnant women. In animals, in utero exposure to BPA increases mammary cancer risk.

With the exception of DES daughters, identification of women who have been exposed to excessive pregnancy estrogenic environment is a challenge, mostly because the time difference between the in utero period and the age when breast cancer is detected is several decades. This is even more difficult if the breast cancer-increasing estrogenic exposure occurred when the great grandmother was pregnant. Identification of persistent changes caused by in utero estrogenic exposures, preferably in the blood, would be a huge step towards identifying the exposed women.

Another challenge is how to prevent high risk women from developing breast cancer. For example, although many DES daughters are aware of their exposure, no specific means are available to prevent these women from developing breast cancer. Additionally, our findings obtained in rats suggest that they may develop resistance to TAM 4-times more frequently than do individuals exposed to normal pregnancy estrogenic environment (FIG. 27). Currently, TAM and raloxifene are the only FDA approved compounds used to reduce ER+ breast cancer among high risk individuals. Thus, novel approaches to prevent and treat breast cancer in women identified as having been exposed to excessive in utero estrogenic environment are needed.

The idea that alterations in miRNAs in the circulation serve as biomarkers of a specific disease, including breast cancer, has recently generated a lot of interest miRNAs are remarkably stable, and changes in the circulating miRNAs generally reflect those seen at the tissue level. Consequently, emerging evidence indicates that different diseases, even different cancers, induce a unique miRNA fingerprint. It is proposed that a miRNA fingerprint indicative of in utero estrogenic exposures and increased breast cancer risk exists in the blood.

Individuals who have been exposed to an excessive in utero estrogenic environment, or are daughters or granddaughters of in utero estrogen exposed mothers or fathers, can be studied to whether such individuals exhibit suppression of E2-regulated miRNAs in the blood. The E2-suppressed miRNAs identified in MCF-7 cells and rat mammary glands target specific polycomb genes that maintain stem cell identity by repressing transcription of Polycomb Target Genes (PcTGs) and consequently inhibit either lineage specific differentiation, and/or enzymes that induce and maintain DNA methylation (DNA methyltansferases: DNMT1, DNMT3a, DNMT3b). One of the key polycombs is EZH2, a histone methyl transferase, which is involved in methylating lysine-27 on histone-3 (H3K27me3), leading to recruitment of DNMTs and causing subsequent DNA methylation. Expression of EZH2 is regulated by miR-26a and miR-101 (35), and DNMTs are regulated by miR-148 and miR-152 (DNMT1), miR-194 (DNMT3a) and miR-26a/b (DNMT3b) (TargetScan 5.1). Expression of each of these miRNAs is suppressed in the mammary glands of rats exposed to EE2 or high fat diet in utero (FIG. 28). Further, our preliminary data show that in utero EE2 and high fat exposures up-regulate DNMT1 (FIG. 30), and an exposure to DES in utero led to an increase in EZH2 expression. Thus, not only are miRNAs persistently down-regulated in the mammary glands in these animals, but the expression of their target genes, some of which are involved in regulating epigenetic events, is altered.

Since alterations in miRNA are likely to be causally related to increased breast cancer, their “normalization” may prevent cancer. Although it is not known the mechanisms involved in persistently suppressing miRNAs in the mammary glands of adult rats exposed to EE2 or high fat diet in utero, they may include DNA methylation. Our preliminary data, using MBDCap-seq, a methyl-CpG binding domain-based capture method coupled with massively parallel sequencing, show that in the mammary glands of both in F1 and F2 generation offspring of EE2 exposed dams (brown bars) (F3 has not been studied yet), DNA methylation in mRNAs and miRNAs across all 21 chromosomes is clearly higher than in the controls (red bars) (FIG. 31). Five regions were associated with polycomb target genes (PcTGs) that are methylated in stem cells (17-19) and breast cancer cells but not in normal mammary tissues (Pax6, Runx3) (20-22), where their hypermethylation is also identified as a hallmark of cancer (Foxe3, Gata4, Veg) (23). Some of these PcTGs, such as Runx3, are reported to be suppressed by E2 (24) and our study indicates that this may be initiated already in the fetal mammary gland and germ cells. Hence, methylation of (breast) cancer specific PcTGs may originate from exposure to excess estrogens in utero (130-135).

### Example 8

### Preclinical Studies to Identify miRNAs in the Blood that May be Indicative of in Utero Estrogenic Exposures

Animal studies are performed to determine the key miRNAs in the blood, which when suppressed, are indicative of an individual's exposure to an excessive estrogenic environment in utero, or through grandmother or great grandmother. This will be accomplished by comparing miRNA patterns in the mammary glands and blood during different stages of carcinogenesis, including before any malignant changes have occurred and during transformation in F1-F3 generations. The Applied Biosystems TaqMan Low Density Array platform is used to generate miRNA profile and determine changes in the expression among different groups. This platform was been validated for human samples, and used to identify differences in miRNA expression in the adult mammary glands of in utero E2 or high fat diet exposed and control rats. The miRNAs are assayed to determine which of them are methylated. Pregnant rats and mice will be exposed to EE2, high fat diet, or the endocrine disruptors BPA and DES, and their F1-F3 generation offspring will be studied.

Identification of miRNA signature in the blood indicative of having been exposed to excessive in utero estrogenic environment in F1 generation offspring and determination as to whether the signature is maintained in F2 and F3 generations is determined using newly developed computational methods, including motif-directed network component analysis (mNCA), multilevel support vector regression (ml-SVR), and differential dependency network analysis (DDN).

Functional consequences of down-regulated miRNAs will also be assessed with the focus on studying changes in polycombs and their target genes (PcTGs), and determining whether maternal estrogenic exposures inhibit lineage specific mammary epithelial cell differentiation, and perhaps increase the presence of stem/progenitor cells.

Reversing Down-Regulation of miRNAs and an Increase in Mammary Tumorigenesis.

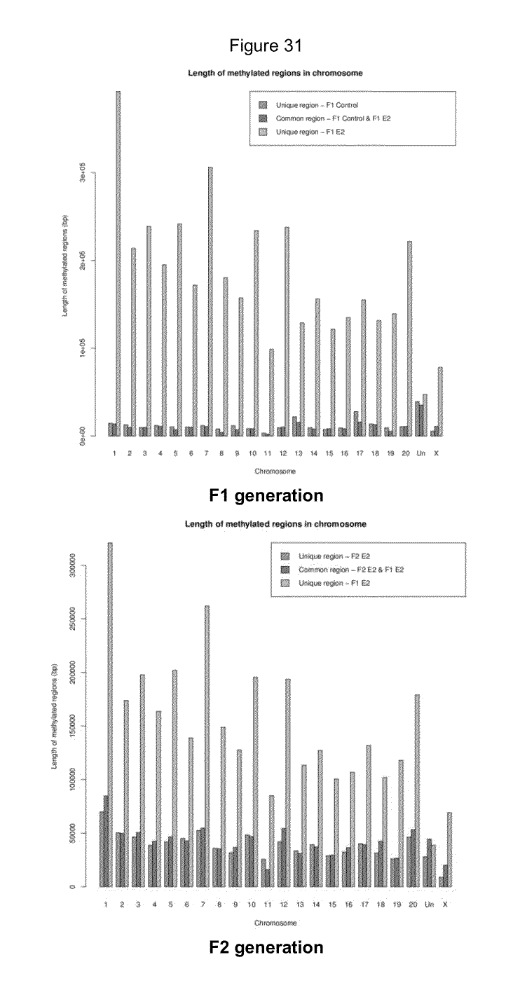

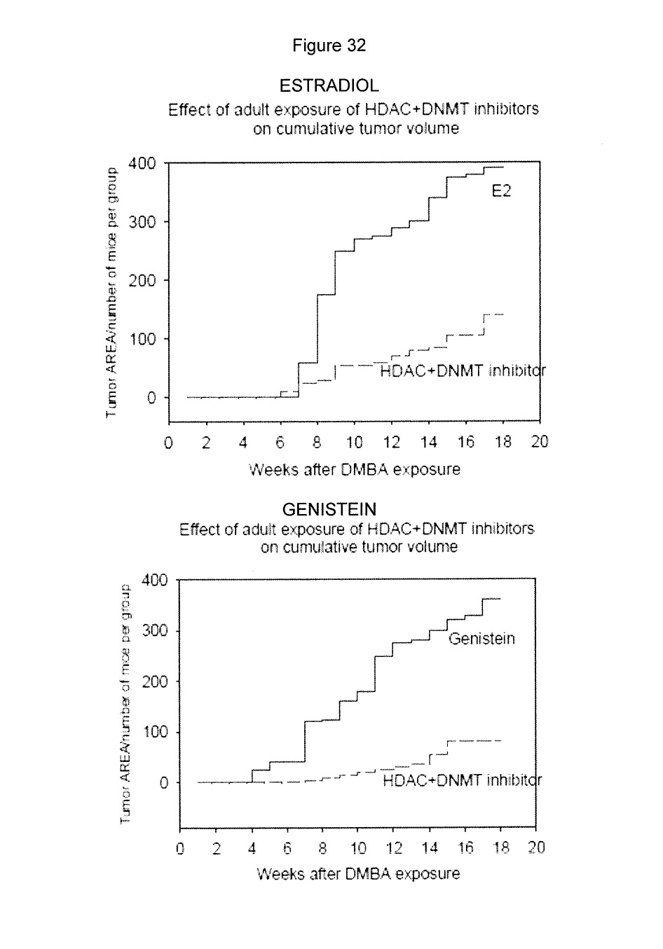

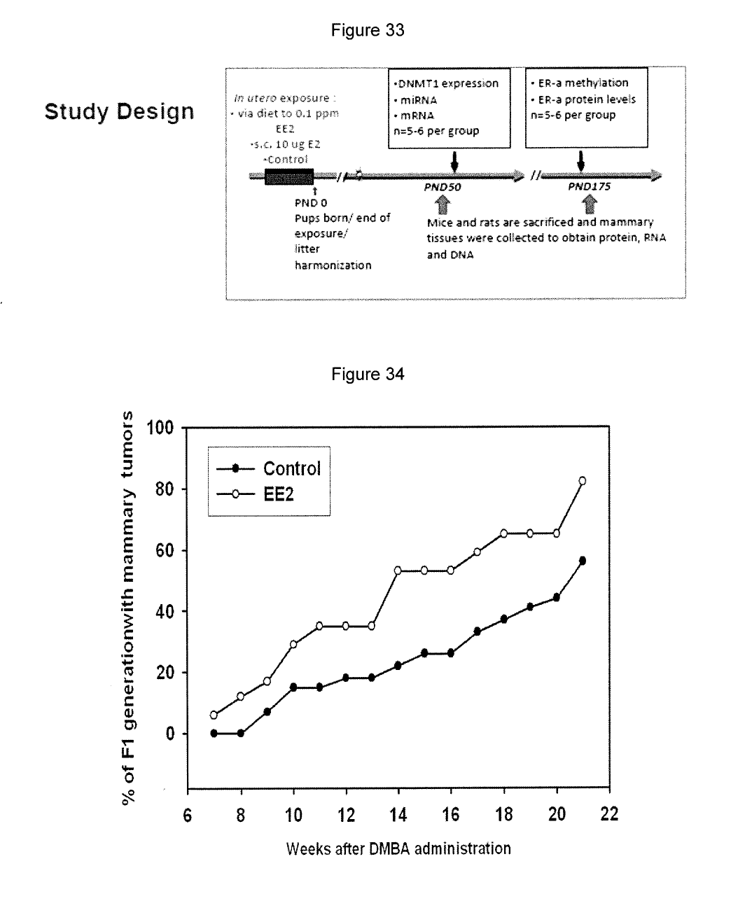

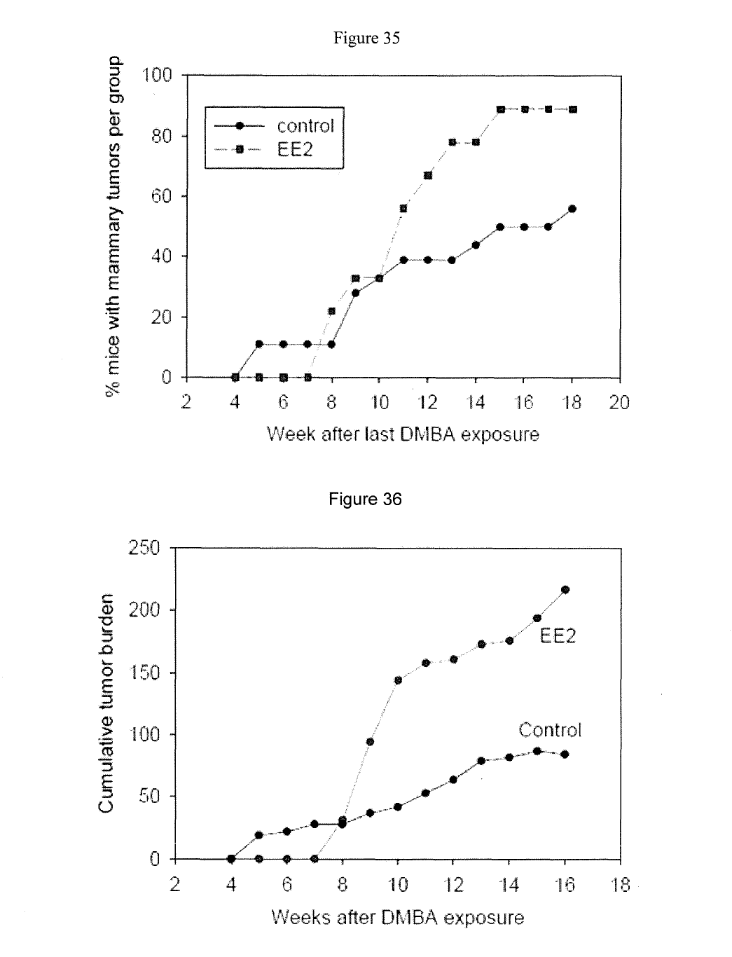

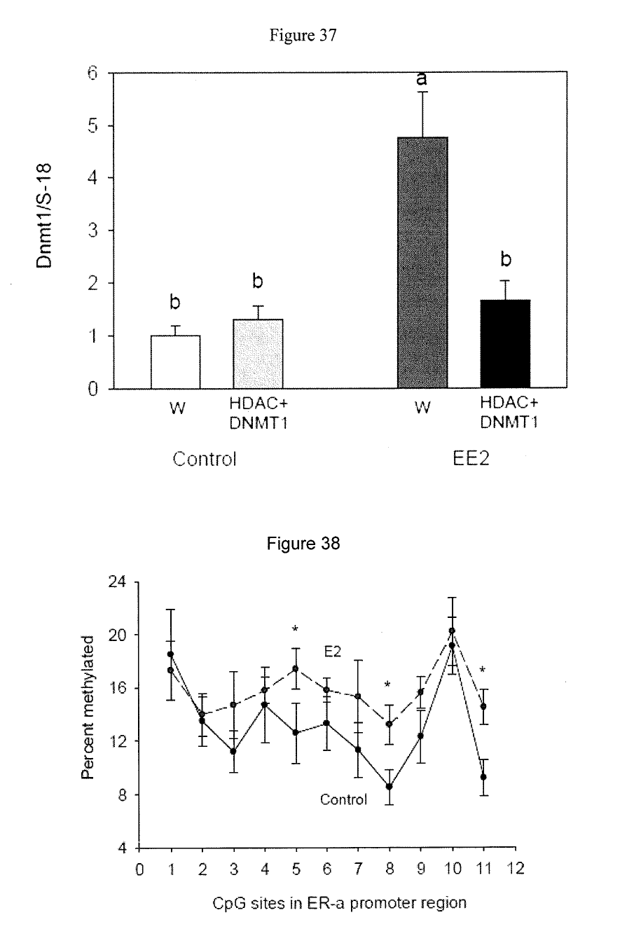

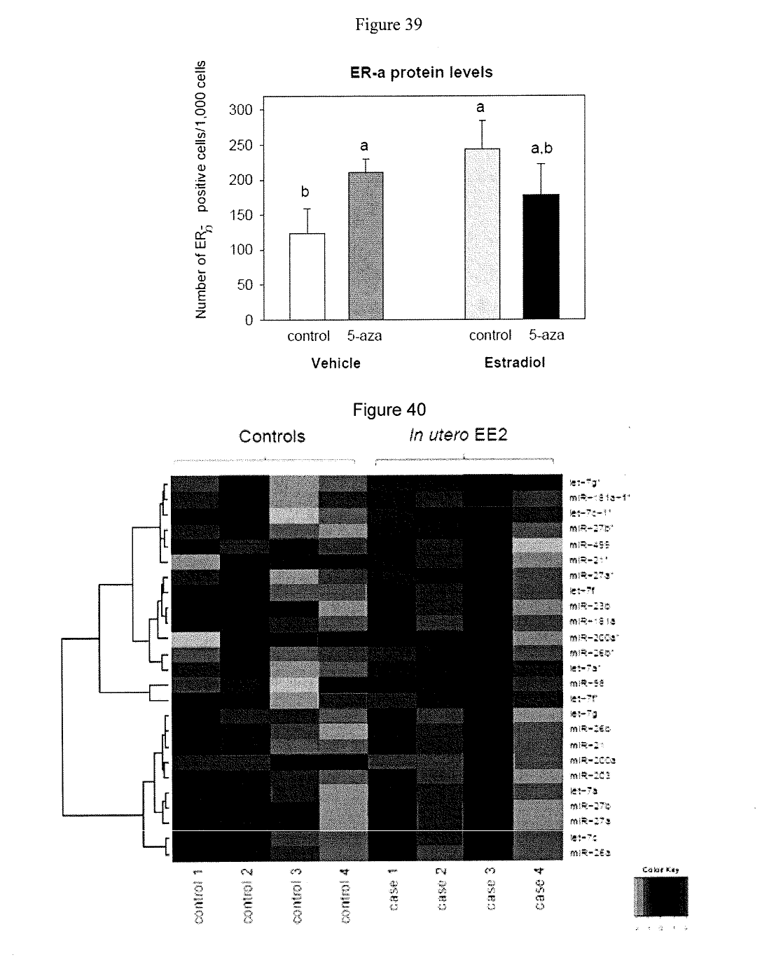

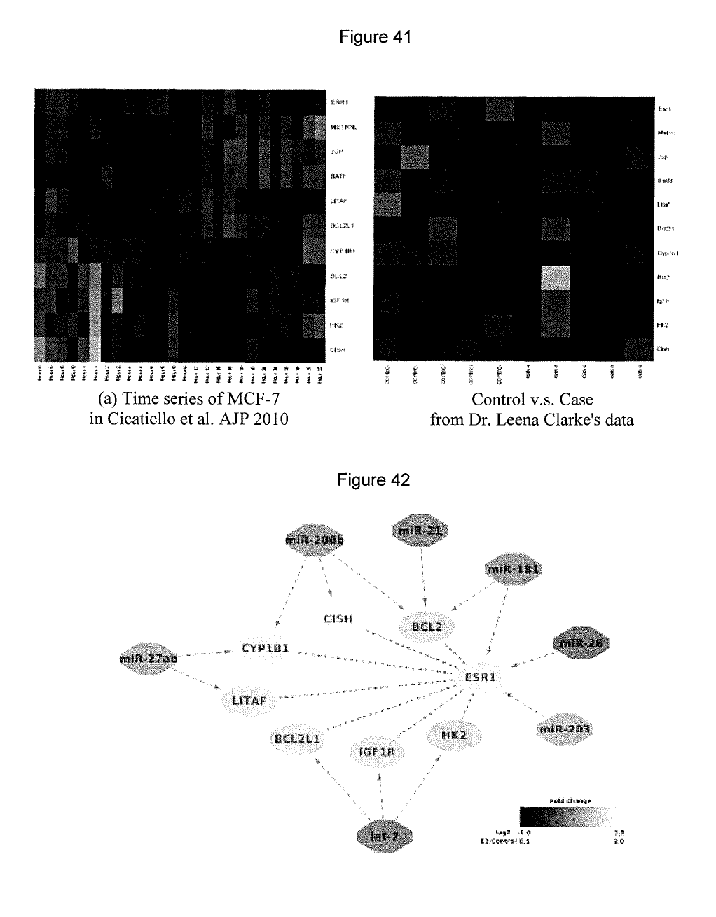

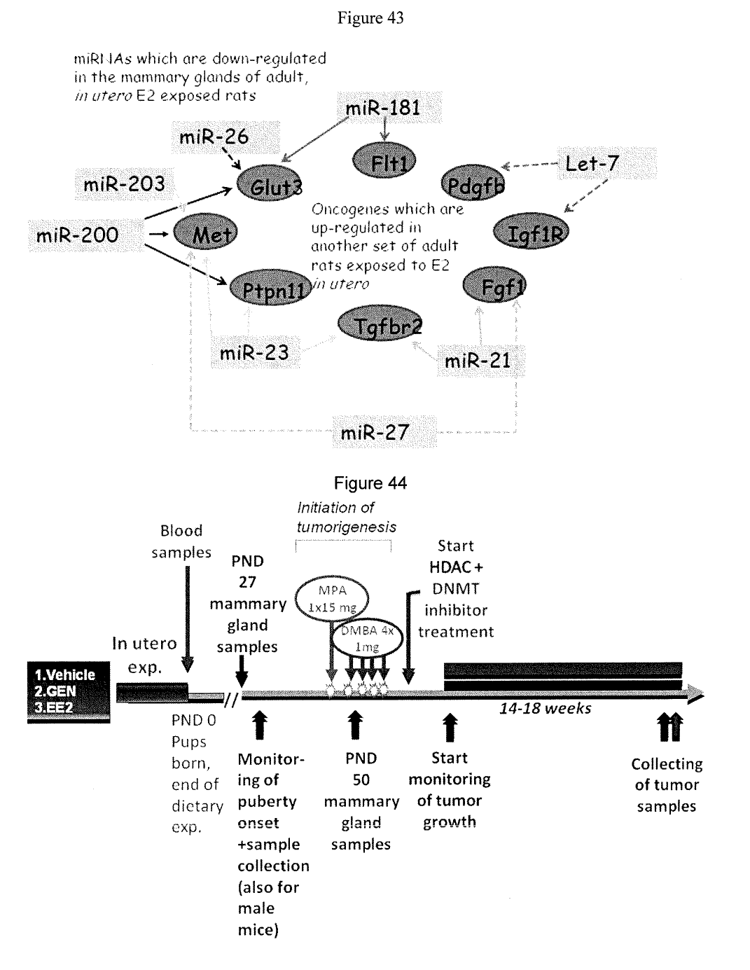

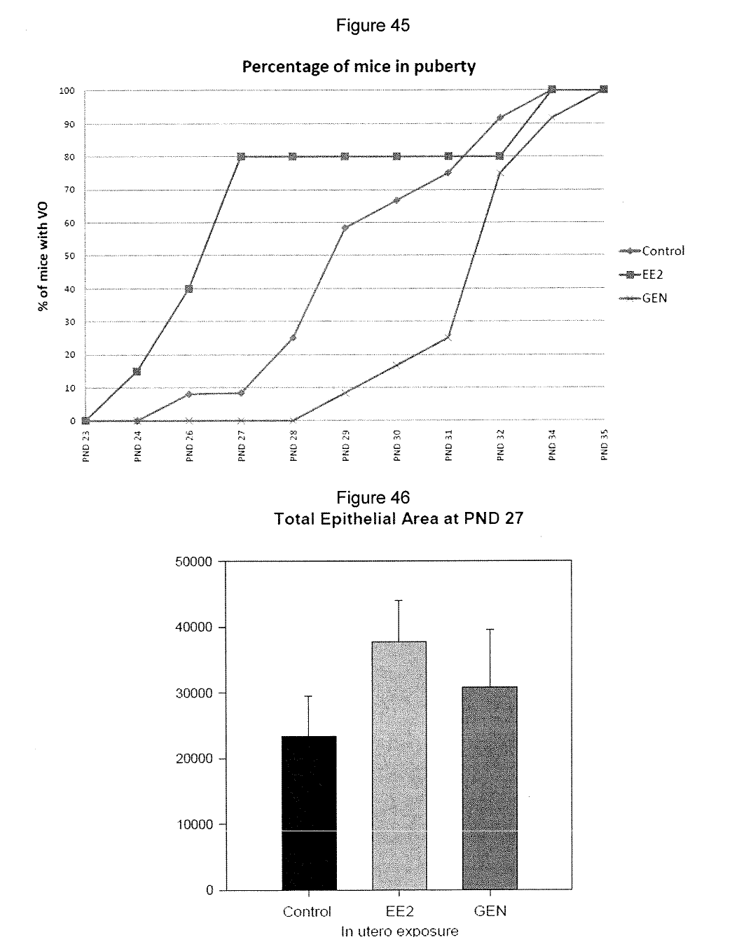

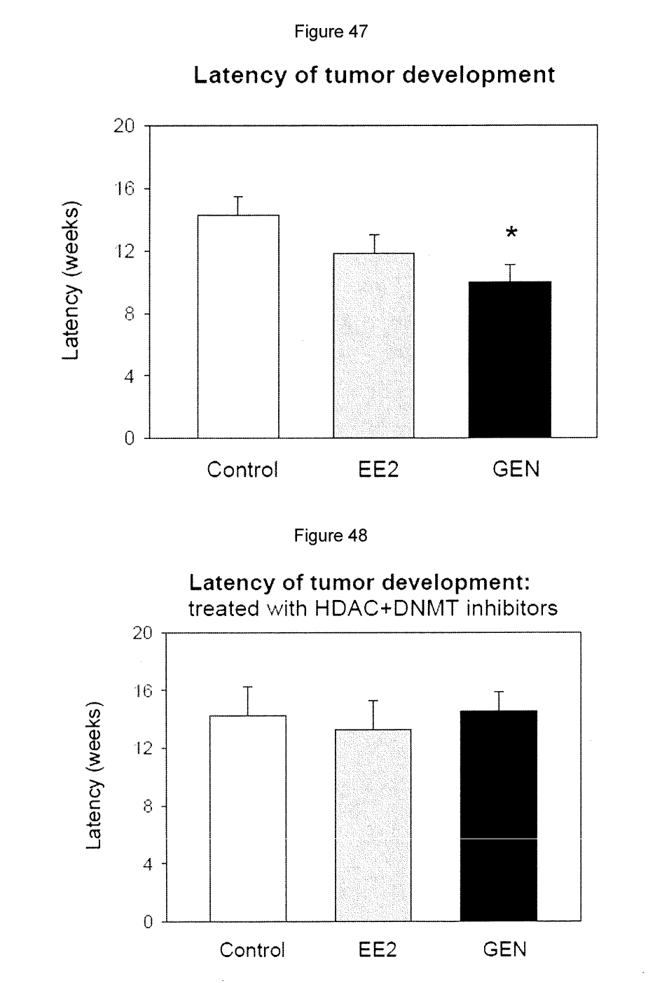

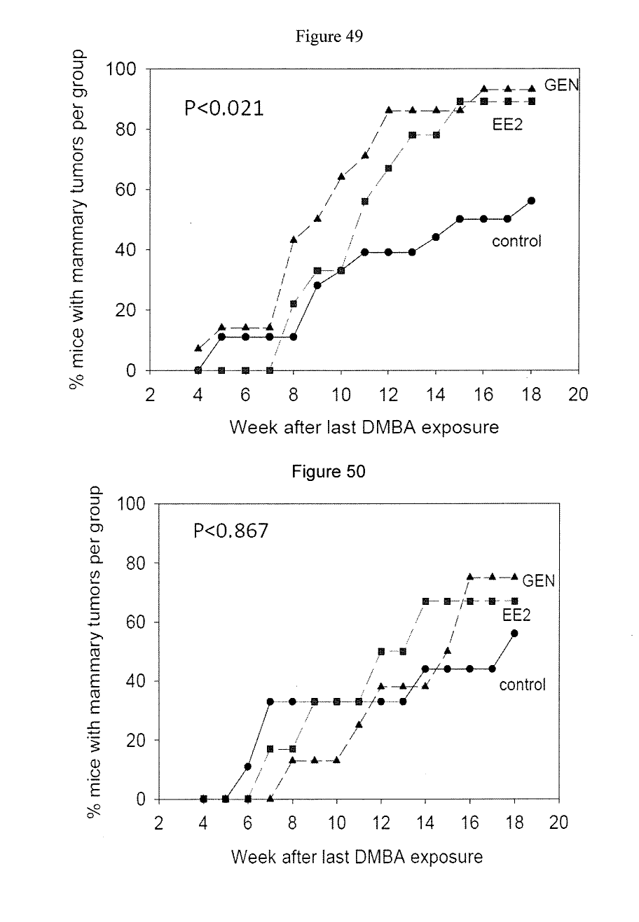

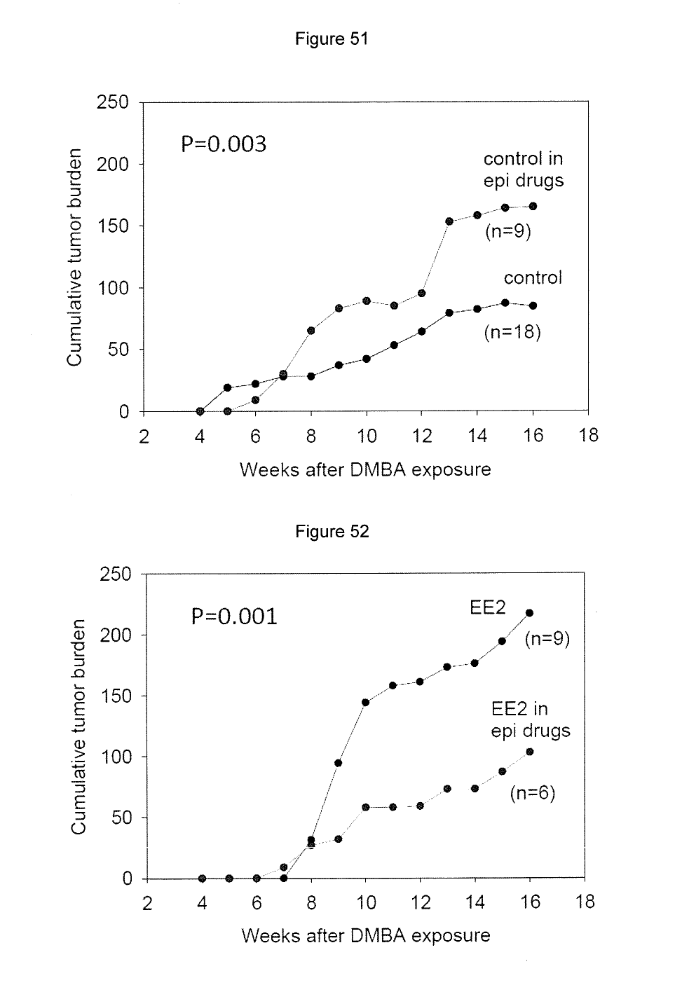

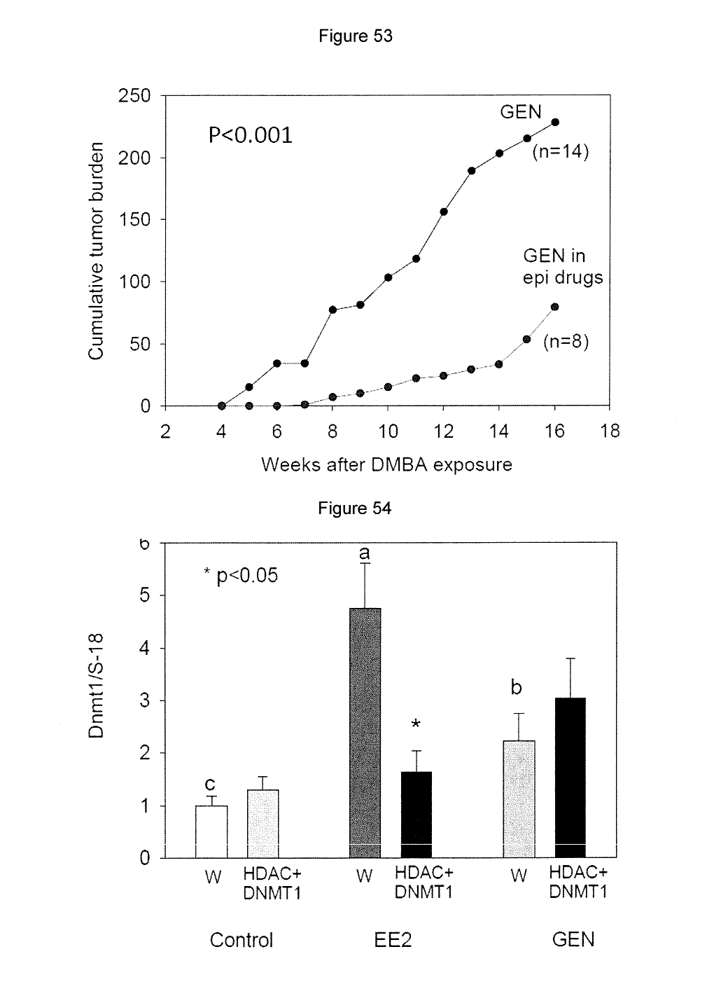

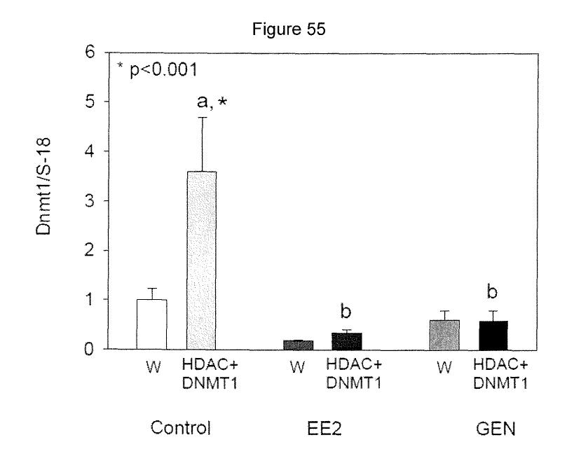

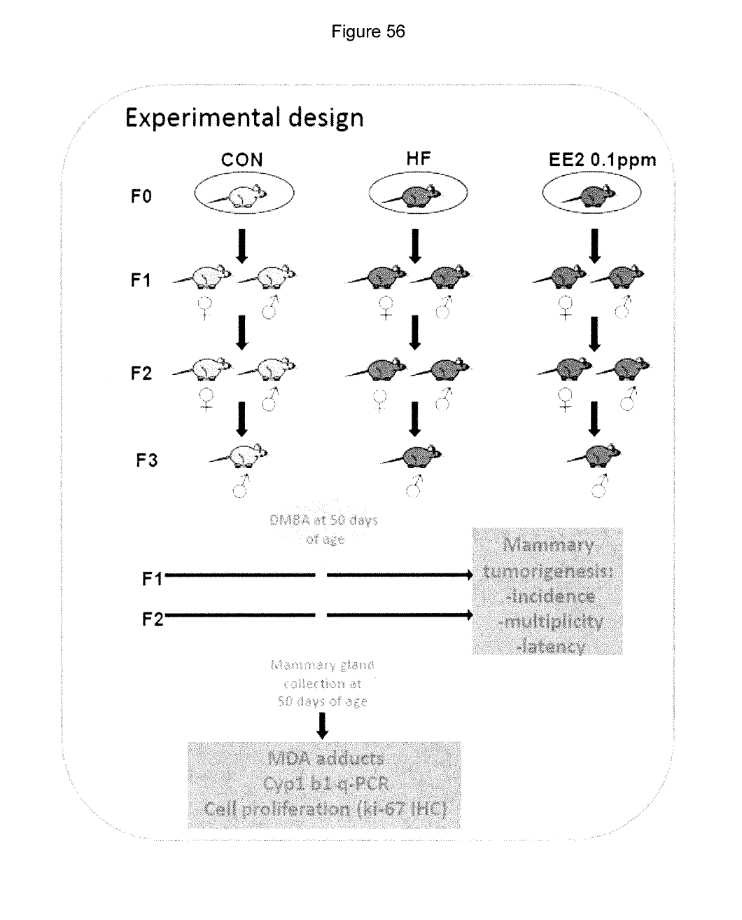

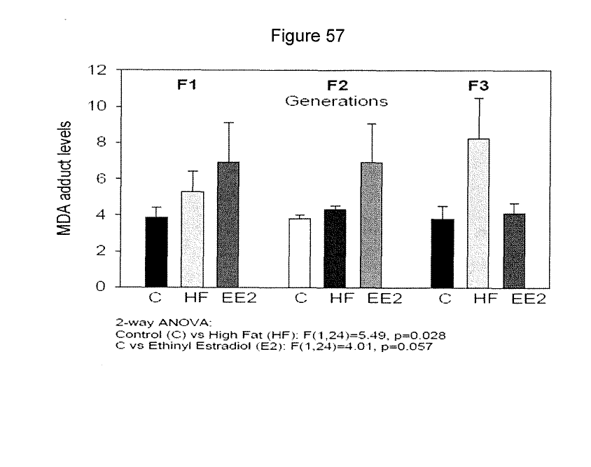

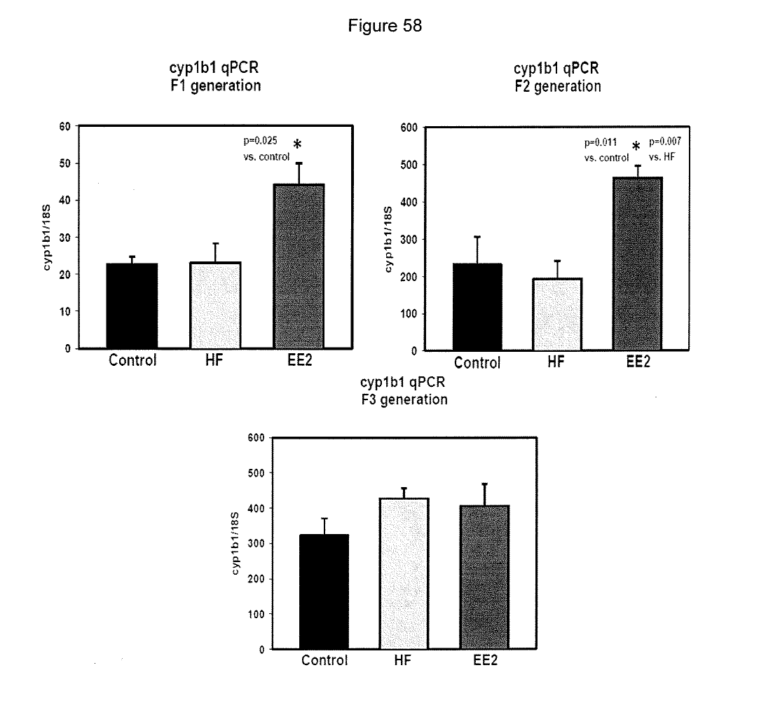

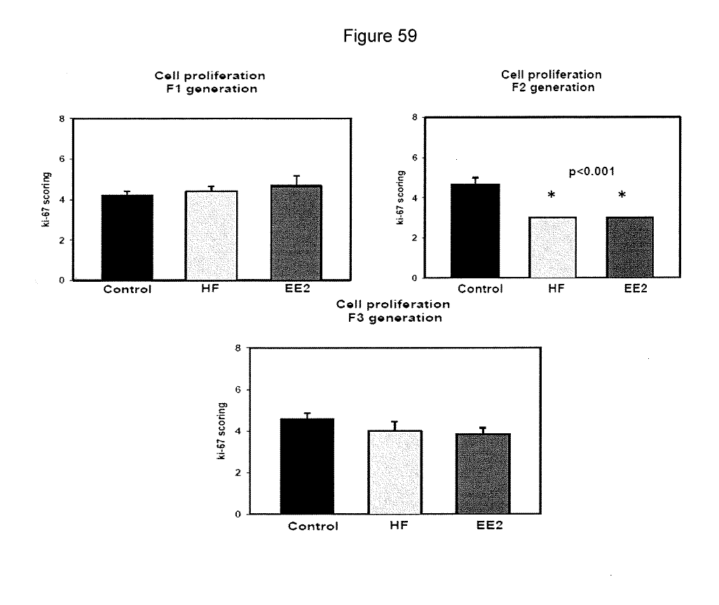

In a preliminary study, the increase in mammary tumorigenesis in mice exposed in utero to EE2 or genistein was successfully reversed by treating them as adults with HCAD inhibitor valproic acid (anti-epileptic drug) and DNMT inhibitor hydralazine (antihypertensive drug) (FIG. 32). These two compounds are well-tolerated and successfully used in combination to treat solid tumors. In addition to using these same compounds, other HDAC and DNMT inhibiting compounds that are currently under clinical trials, particularly those used for breast cancer patients, are explored. It will be investigated whether these treatments reverse down-regulation of specific miRNAs and increased mammary tumorigenesis in F1-F3 generation offspring of dams exposed to excessive pregnancy estrogenic environment. Whether the treatments promote lineage specific differentiation of mammary stem/progenitor cells, because miRNAs suppressed in the offspring's mammary gland target polycombs which inhibit stem/progenitor cell differentiation, can be evaluated.

### Preclinical Studies on the in Utero Estrogenic Exposures and Antiestrogen Resistance.

It is known that in utero E2 exposure increases the risk of acquired TAM resistance from 7% in the control rats to 50% in the in utero E2 group (FIG. 27). Whether this is also seen in the offspring exposed to DES and whether it persists to F2-F3 generations can also be investigated. In addition, it has been shown that it is possible to culture the tumors exhibiting acquired resistance using a new culture system developed by Dr. Richard Schlegel, and the AE resistant tumors obtained from the in utero E2 exposed rats maintain ER+ and remain TAM unresponsive. The culture system is used to identify transcription factors and/or miRNAs that are involved in the development of TAM resistance and determine whether reversal of the changes will reverse TAM resistance. Compounds which reverse the changes in vitro are analyzed to determine if they are effective in pre-clinical animal model. The same model that was used to discover that TAM inhibits breast cancer is used.

Clinical Studies on the Identification of in Utero Estrogenic Exposure miRNA Signature in the Human Blood.

Pre-clinical studies will be able to identify miRNA signature in the blood of F1-F3 generation offspring of dams exposed during pregnancy to EE2, high fat diet, BPA or DES, and if this signature and increased mammary cancer risk can be reversed by treating animals with HDAC and DNMT inhibitors, we will start to perform clinical studies to determine whether such a signature can be seen in (a) DES daughters or granddaughters, and (b) women at high familial risk for developing breast cancer, but who are negative for germline BRCA1/2 mutations. The studies are limited to involve women who are under 50 and have not been diagnosed with breast cancer. Methylation status of the down-regulated miRNAs will be also determined.

Women exhibiting in utero estrogenic exposures miRNA signature will be invited to participate a clinical study in which they are treated with HDAC and DNMT inhibitors that are safe and effective in breast cancer patients. Women are kept on these compounds for few weeks, and their ability to reverse in utero estrogenic miRNA signature will be investigated.

An epidemiological study can compare TAM resistance in breast cancer patients who had high familial risk or who were exposed to DES in utero to an appropriate control women.

### Description of DES Cohort.

As part of the NCI collaborative study of DES health effects, Dr. Palmer has followed a cohort of women exposed to DES in utero and a comparison cohort of unexposed women since 1994. Over 1100 DES-exposed and 600 unexposed women have completed questionnaires every five years. The majority of study subjects live in Massachusetts.

### Description of High Familial Cancer Registry (FCR).

The FCR includes over 2,500 individuals (with or without cancer) at high familial risk for breast cancer, the majority of whom have undergone genetic testing. Currently, the FCR actively follows over 700 women who have tested negative for BRCA1/2 mutations. This comprehensive resource includes annually updated family and personal medical history, detailed epidemiological data, pathology reports, as well as bio-specimens. Participants in FCR consent to be re-contacted for future studies.

The Anticipated Effects on the Prevention of Breast Cancer if the Project is Successful, and How the Findings would be Transformative

This project is expected to lead to a development of blood-based biomarkers identifying those women who are at increased risk of developing breast cancer due to having been exposed to excess estrogens in utero (or the exposure occurred through grandmother or great grandmother), and novel, safe strategies to reduce their breast cancer risk. It is not known how many women are exposed to elevated pregnancy estrogenic environment, but over 22% of pregnant women have levels which are 1.5-fold higher than mean levels, 5-10 million pregnant women have been exposed to DES, and exposure to estrogenic EDCs during pregnancy is common. The project may also lead to identification of a subset of women (exposed to excessive in utero E2 environment) with ER+ breast cancer who are at increased risk of developing TAM resistance.

Describe the Challenges Associated with Implementing Your Vision—What Barriers have to be Overcome?

While miRNAs have been identified that are persistently down-regulated in the mammary tissue following in utero exposure to an excessive estrogenic environment, it we have not determined whether similar changes are seen in the blood. However, since several recent studies indicate that changes in the miRNAs in the peripheral blood closely mimic those seen at the tissue level, we are confident that some miRNAs are similarly altered in the mammary gland and blood in animals (and women) exposed to excessive estrogenic environment in utero. Further, even if we fail to find a high correlation between changes in miRNAs in the mammary tissue and blood, blood miRNAs can still be used to identify in utero estrogen exposure signature.

Since we are searching for biomarkers of excessive pregnancy estrogenic exposures in women before cancer develops, we do not expect to have mammary tissues available from these women. Thus, pre-clinical studies to be performed during years 1-2 play a critical role in validating the correlation between changes in the tissue and blood. Furthermore, the pre-clinical studies, combined with data modeling, can provide information as to whether changes in miRNAs can be reversed both in the mammary tissue and blood by using HDAC+DNMT inhibitors, and whether these also contribute to reversal of changes in stem cell behavior and the increase in mammary cancer risk we have previously observed in mice exposed to EE2 or genistein in utero (FIG. 6).

Finally, in this study we are searching for changes in miRNA in two groups of women: (1) women who are at high familial risk of developing breast cancer, but are not carriers of BRCA1/2 mutations, and (2) daughters and granddaughters of DES exposed mothers. We do not expect that all women at high familial risk have been exposed to an excessive in utero estrogenic environment, and this reduces our ability to identify miRNA fingerprint indicative of the exposure. However, since the DES daughters have been exposed to a synthetic estrogen in utero, and we will compare miRNA changes in the blood of these women to those in the blood and mammary gland of in utero DES, EE2 or high fat exposed pre-clinical models, we are confident that we will determine whether some of the high familial risk women have been exposed to excess estrogens in utero.

### Example 9

### Elevated in Utero Estrogenic Environment May Increase Later Breast Cancer Risk by Down-Regulating miRNAs

A high in utero estrogenic environment increases the risk of developing breast cancer (FIG. 1). Increased risk is associated with greater epithelial cell proliferation and survival during mammary gland development, driven by persistent changes in the mammary transcriptome. How these transcriptional changes are initiated and maintained is unclear. We first investigated whether in utero exposure to excess estradiol (E2) or ethinyl estradiol (EE2) alters the expression of DNA methyltransferase 1 (DNMT1) which, when up-regulated, is associated with silencing of genes by DNA methylation. DNMT1 was up-regulated in the mammary glands of mice and rats exposed to 0.1 ppm EE2 in utero (FIG. 2). We then determined whether estrogen receptor alpha (ER-a) is methylated in the E2 exposed rats. FIG. 3 shows that this was the case. Several other genes, particularly polycomb group target genes (PcGTs) which are commonly methylated in peripheral DNA in women at high breast cancer risk, were also down-regulated in the mammary gland of in utero E2 exposed rats (Table 1). However, expression of ER-a was up-regulated in the mammary glands of in utero E2 exposed rats (FIG. 4), suggesting that other mechanisms are involved in causing methylated ER-a gene to be overexpressed. These mechanisms could include non-coding microRNAs (miRNAs) which are essential for early development and which inhibit expression of their target genes. Since several miRNAs are known to be suppressed by E2 in MCF-7 human breast cancer cells (Maillot et al. Cancer Res 2009), we determined whether persistent changes in miRNA expression may be seen in the adult mammary glands of rats exposed to E2 in utero. miRNAs were measured using applied Biosystems TaqManR Array Rodent MicroRNA. As shown in FIG. 5, several miRNAs were down-regulated in these rats, and most of them are the same miRNAs than those seen to be down-regulated in the MCF-7 cells. We next studied whether there is an overlap between up-regulation of mRNAs identified using RG_U34A Affymetrix platform, and down-regulated miRNAs. FIGS. 6 and 7 show that many target genes of E2 down-regulated miRNAs were up-regulated, including down-stream targets of ER-a and oncogenes. Our findings suggest that maternal exposure to excess E2 during pregnancy causes a persistent up-regulation of ER-a and other estrogen-regulated genes in the offspring's mammary gland by suppressing miRNAs that target these genes. In addition, some of these miRNAs are known to target DNMTs, and this could explain a persistent up-regulation of DNMT1.

### Example 10

### Prevention of High Breast Cancer Risk in Mice Exposed to Endocrine Disrupting Chemicals in Utero by Adult Exposure to HDAC and DNMT Inhibitors

Prevention of high breast cancer risk in mice exposed to endocrine disrupting chemicals in utero by adult exposure to HDAC and DNMT inhibitors
Objective: To test a hypothesis, that increased mammary cancer risk of animals exposed to excessive in utero estrogenic environment is caused by epigenetic changes, and can be reversed by erasing these changes in adulthood with DNMT and HDAC inhibitors
This study was designed to investigate a hypothesis that exposures to endocrine disrupting chemicals (EDCs) early in life increase later susceptibility of developing breast cancer by inducing heritable epigenetic changes, including in tumor suppressor genes and other transcription factors which drive malignant transformation. For this purpose, pregnant mouse dams were exposed to either synthetic estrogen ethinyl estradiol (EE2) or genistein (GEN), both of which are previously reported to increase mammary tumorigenesis among female offspring. Results show that in utero exposures to EE2 or GEN did not alter birthweight, or postnatal weight development Female offspring of EE2 exposed dams exhibited significantly earlier puberty onset, but the GEN exposed females exhibited delayed vaginal opening. Mammary gland morphology was altered: exposed mice had significantly higher number of terminal end buds (TEBs); i.e., structures where malignant transformation takes place, and mammary parenchymal area was significantly enlarged (in the genistein group, the increase did not reach statistical significance). Assessment of mammary tumorigenesis indicated that both in utero EE2 and GEN offspring exhibited increased mammary tumorigenesis, compared to the controls. Most importantly, an exposure to epigenetic compounds in drinking water in adulthood; i.e., histone deacetylase (HDAC) inhibitor Valproic acid (1.16 g/kg/day) and DNA methyltransferase (DNMT) inhibitor Hydralazine (5 mg/kg/day), which are in use in the clinic to treat various cancers, reversed the increase in mammary tumorigenesis in the EE2 and genistein offspring. In addition, mammary glands of EE2 and GEN offspring exhibited an increase in DNMT1 expression and the epigenetic compounds reversed the increase in the EE2 group. We are currently investigating whether in utero exposure to GEN also increased HDAC expression and Valproic acid inhibited it. In conclusion, our results indicate that increased breast cancer risk in individuals exposed to EDCs in utero can be reversed by giving them inhibitors of HDAC and DNMT in adult life. An important future task is to establish epigenetic biomarkers in the blood, such as methylated tumor suppressor genes, to be able to identify in utero EDC exposed women to prevent their breast cancer.

### Study Design

### Evaluation of in Utero Endocrine Disruptor Chemical (EDC) Exposure's Effects on Growth and Sexual Maturation of Pups

### Puberty Onset of Female Mice Exposed in Utero to EDC.

Puberty onset is considered an important parameter informing about possible adverse effects of in utero EDC exposure, and in women an early onset of puberty is a risk factor for breast cancer.

### Findings:

In utero EE2 exposure significantly accelerated and genistein exposure delayed vaginal opening.

Growth of the in Utero Exposed Female and Male Pups Prior to their Puberty Onset.

No statistically significant differences in body weights were observed in female pups on PND 27, a few days prior to their puberty onset (FIG. 13).

### Mammary Gland Epithelial Area & TEB Number on PND 27.

Mammary gland morphology of female mice was assessed at PND 27 using wholemount technique to reveal possible changes due to the in utero ECD exposures. Mammary gland growth was clearly affected; all the in utero exposures significantly increased the number of TEBs, the growing points of the mammary epithelium likely to contain (most of the) stem cells in a pubertal mammary gland. This was reflected also in the total epithelial area of the glands, which was significantly increased in the EE2 and BPA exposed female mice, and nearly significantly also in the GEN exposed females.

Increased Mammary Tumorigenesis in Mice Exposed to EE2 or GEN in Utero. Adult Exposure to HDAC and DNMT Inhibitors Reverses the Increase

### Tumor Latency

Maternal Exposure to GEN Reduces the Latency of Mammary Tumor Development. Adult Exposure to HDAC+DNMT Inhibitors Reverses this Effect.

Latency for first tumor to develop was significantly shorter in mice exposed to EDCs in utero, compared to the vehicle treated control mice (p=0.038); the difference was most significant between the control and genistein groups (p=0.012). However, if the mice were treated with HDAC+DNMT inhibitors, no significant differences in tumor latency among the three groups were seen (p=0.908).

### Tumor Incidence

Maternal Exposure to EE2 or GEN During Pregnancy Increases Mammary Tumorigenesis Among Female Offspring. Adult Exposure to HDAC and DNMT Inhibitors Prevents this Increase.

Mammary tumor incidence was significantly higher in mice exposed to EE2 or genistein in utero than in the control mice (p=0.021). The difference was more significant in the genistein group (p=0.009) than in the EE2 group (p=0.09). Treatment of the mice exposed to EE2 or genistein in utero with HDAC+DNMT inhibitors in adult life abolished this difference, compared to the HDAC+DNMT inhibitor treated control mice (p=0.867).

### Tumor Burden

### Adult Exposure to HDAC+DNMT Inhibitors Reverses the Increase in Mammary Tumor Burden in Female Offspring of Dams Fed EE2 or GEN Containing Diet During Pregnancy.

Differences in cumulative tumor burden (size of all tumors per mouse) were assessed by determining total weekly tumor burden in the group, divided by the number of mice in the group. Final tumor burden per mouse was significantly higher in both the EE2 (p=0.039) and genistein (p=0.009) groups, compared to the controls (p=0.018). This difference was not seen among the three groups of mice treated with HDAC+DNMT inhibitors in adult life (p=0.674).

### Permanent Changes in Expression of DNMT1 in Mammary Glands & Tumors Exposed to EDC During Embryogenesis

### Mammary Glands

Expression of DNMT1 was determined both in the mammary glands and tumors in mice exposed to EE2 or GEN in utero. As previously seen in rats, mice exposed to EE2 in utero exhibited an increase in the expression of DNMT1 (p<0.002), and GEN exposed offspring showed a similar increase (p<0.004).

An exposure to HDAC+DNMT1 inhibitors reversed the increase in the EE2 group (p<0.004), but not in the GEN group (F for interaction: p<0.007)

### Mammary Tumors

In the mammary tumors, DNMT1 expression was significantly lower in the mice exposed either to EE2 (p<0.002) or GEN in utero (p<0.004) than in the control mice.

Further, HDAC+DNMT1 inhibitors significantly increased DNMT1 expression in the mammary tumors in the control mice (p<0.001), but not in the EE2 or GEN group (F for interaction, p<0.039).

In this study, we exposed pregnant mouse dams to either synthetic estradiol EE2 or an endocrine disruptor (EDC) genistein. In agreement with earlier findings obtained in rat models, both exposures increased mammary tumorigenesis among female offspring. We also found that:

Consistent with a previous study, in utero exposure to EE2 led to earlier puberty onset. In utero genistein exposure, in turn, delayed puberty onset.

Earlier studies have reported an increase in the number of targets for malignant transformation; i.e., TEBs, in offspring of dams exposed to estradiol or genistein during pregnancy. Findings obtained in this study confirm these previous observations and indicate that the mammary gland is a target of in utero exposure to EDCs.

Elevation of TEBs is, therefore, one biomarker of an exposure to EDCs in utero in mice and rats.

An exposure to the HDAC inhibitor Hydralazine and the DNMT inhibitor Valproic acid in drinking water which started after mammary tumors were initiated prevented the increase in mammary tumorigenesis in the offspring exposed to EE2 or genistein in utero.

In utero exposure to EE2 and GEN caused a persistent up-regulation of DNMT1 in the normal mammary gland, and this increase could be prevented by adult exposure to HDAC+DNMT1 inhibitors in the EE2 group, but not in the genistein offspring.

Our results have two important implications:
(1) in utero estrogenic exposures increase later breast cancer risk via epigenetic mechanisms.
(2) increased breast cancer risk in EDC daughters may be reduced by HDAC and DNMT1 inhibitors.
These findings provide the first experimental evidence indicating that the increase in breast cancer risk induced by an exposure during fetal development to excessive estrogenic environment/EDcs can be prevented by adult exposure to compounds which inhibit HDAC and DNMT

### Example 11

### Maternal Exposure to a High Fat Diet or Estradiol During Pregnancy Increases CYP1B1 Expression and DNA Adducts in the Mammary Glands of F1-F3 Generation Offspring

Maternal exposure during pregnancy to estradiol (EE2) or a high fat (HF) diet, which increases circulating estrogen levels, increases mammary cancer risk in daughters (F1), granddaughters (F2), and great-granddaughters (F3) in Sprague Dawley rats. This study investigated whether the increase in mammary tumorigenesis in the F1-F3 offspring is associated with increased expression of estrogen metabolizing enzyme CYP1B1 in the mammary glands of 50-day-old offspring and consequently formation of DNA adducts. CYP1B1 belongs to the cytochrome P450 superfamily of enzymes. In the F1 and F2 generations, expression levels of CYP1B1 were significantly higher in the EE2 than in the control or HF offspring. In the F3 generation, CYP1B1 expression levels in EE2 and HF were higher than in the controls, with the HF offspring exhibiting the highest level of expression. Assessment of DNA adduct formation by measuring MDA levels mimicked the findings of changes in CYP1B1 expression. MDA is malondialdehyde, which is structurally represented as

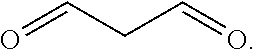

Possible changes in cell proliferation by ki-67 in the mammary glands of all groups were assessed. In the F1 generation, cell proliferation in the HF and EE2 was non-significantly increased, but in the F2 generation, cell proliferation in both HF and EE2 were significantly lower compared to the controls, perhaps in response to increased DNA damage. Our result show that increased expression of CYP1B1 might be a vital factor in in utero estrogenic exposure-mediated mammary carcinogenesis to induce DNA damage. We are currently investigating whether the increased expression of CYP1B1 is caused epigenetically by reduced expression of non-coding miR-27 and miR-200 that target this estrogen metabolizing enzyme.

### Introduction

One out of 8 women in the U.S. are expected to develop breast cancer in their lifetime. Studies have shown that diet is one of the factors that causes cancer and that it plays a role in predisposition to this disease. Dietary components, such as fats and phytoestrogenes, have been shown to affect enzyme activity and alter estrogenic environment, which in turn can influence gene expression. The timing of exposure to components that change estrogenic environment have been shown to influence breast cancer risk. In addition to the mendelian mode of DNA inheritance, epigenetic mechanisms are being considered as a mean of explaining the transmission of breast cancer risk in some families. Epigenetics is the inheritance of changes in gene expression that does not involve changes in the DNA sequence, and include DNA methylation, histone modifications and non-coding microRNAs. These epigenetic alterations may be transgenerationally inherited, transmitted through the germline, which then influences breast cancer risk from one generation to the next. In this study, maternal exposure during pregnancy to excess estradiol or high fat diet not only increased breast cancer risk in the daughters (F1), but also subsequent generations (F2 and F3; granddaughters and great-granddaughters, respectively. The levels of MDA-DNA adducts were increased in F1-F3 generation offspring. Consequently, we investigated whether the increase in MDA-adducts seen in F1-F3 generations may be due to alterations in the expression of enzymes involved in estrogen metabolism. In the metabolism of estrogen, human cytochrome P450 (CYP1B1), is an important enzyme that hydroxylates 17β-estradiol (E2) to form 4-hydroxyestradiol (4-OHE2) at the C-4 position. 4-OHE2, a catechol estrogen, can then be oxidized to quinine, which initiates tumor formation. In normal metabolism of estrogen, CYP1B1 is down-regulated, however, due to evidence which shows that in utero exposures to excess estrogens increase carcinogen-induced mammary tumorigenesis in rat models, CYP1B1 might be up-regulated in the normal mammary glands of F1-F3 generation offspring.

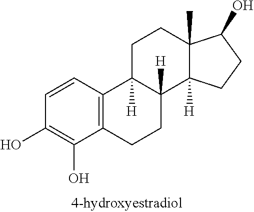

cDNA Synthesis and Quantitative Real-Time PCR (qRT-PCR) Analysis:

Mammary gland tissue obtained from 50-day-old F1-F3 generation offspring (n=5-6 per group per generation) were used. 1 μg of total RNA per sample was used as a template for random primed cDNA synthesis. The cDNA samples were used as templates for qRT-PCR analysis with specific primers for the target gene, rat CYP1B1 using QuantiTect SYBR green PCR kit (Qiagen Inc., Valencia, Calif.) and an ABI Prism 7900 Sequence Detection System. Each sample was run in triplicate. Absolute gene expression levels were determined using Applied Biosystems' SDS2.3 software and the standard curve method. Concentration of each sample was normalized to the reference gene 18S rRNA.

### Cell Proliferation:

To determine whether the increase in DNA adduct formation may reflect increased cell proliferation, expression of Ki-67, a marker of cell proliferation, was assessed on PND50 mammary glands using immunohistochemistry. The ki-67 status of each sample was evaluated according to a modified version of the scoring system proposed by Allred et al. The total score for each sample was calculated as the sum of the estimated proportion of positive-staining cells (0-7) plus the estimated intensity of positive-staining cells (0-4).

The results of this study showed that CYP1B1 expression is up-regulated in the normal mammary glands of F1-F3 generation offspring of rat dams exposed to EE2 during pregnancy. In the HF offspring, the increase in CYP1B1 levels was seen only in the F3 generation. These results parallel the results seen in the MDA-DNA adduct study. It was also found that cell proliferation was significantly reduced in the F2 generation offspring, and tended to be reduced in the F3 offspring, perhaps in response to increased DNA damage. Our future studies will determine whether the increase in CYP1B1 expression is epigenetically induced by down-regulation of miR-27 and miR-200 that target this enzyme.

Down-Regulation of DNMT/HDAC and miRNAs

The methods of the present invention provide a reduction in the cancer risk for individuals exposed to an elevated in utero estrogenic environment. Cancers treated by the invention include cancers of the endocrine system, an exemplary cancer of the endocrine system is breast cancer. An elevated in utero estrogenic environment could be caused by an in utero exposure to diethylstilbestrol (DES), endocrine disrupting chemicals (EDC) or dietary factors which increase estrogen levels, including a high fat diet. One method of the invention involves the treatment of individuals at a high-risk for cancer with compounds that erase the epigenetic changes that may have taken place due to exposure to an elevated in utero estrogenic environment. Without being bound by any particular theory, it is believed that epigenetic changes form the memory mark and mediate the effects of in utero estrogenic exposures on later cancer risk, perhaps including breast cancer. All cells in different tissues and organs in humans and other multi-cellular organisms originate from a single fertilized cell and thus these cells have identical DNA sequences, although they are morphologically very different and have different functions. Cell differentiation to multiple lineages is governed by both genetic and epigenetic changes, and the latter is influenced by the environment in utero. These early epigenetic processes include DNA methylation and histone modifications. In simple terms, the level of DNA methylation is determined by the presence of methyl groups in CpG islands located mostly in gene's promoter region. The more methylated the islands are, the less the gene can be expressed. These methylation patterns are then inherited to daughter cells by mechanisms which involve DNA methyltransferases (DNMTs). DNA methylation patterns in different cells during fetal development can be modified by maternal exposure to EDCs, but we are not aware of any published studies that have investigated the effect of in utero EDC exposure on DNA methylation in the breast.

Epigenetic changes are thought to be reversible. These changes are common in different cancers, and consequently some cancers are treated with DNMT inhibitors, often in combination with histone deacetylase (HDAC) inhibitors. However, it is not clear what causes DNA methylation of, for example, tumor suppressor genes (TSGs) or polycomb target genes (PcTGs), although their DNA methylation in cells of peripheral blood or breast fluid before any cancers are detected, is strongly predictive of increased breast cancer risk. We have found, and describe here, that in utero estrogenic exposures cause down-regulation of both TSGs and PcTGs. Table 1 shows PcGTs which are down-regulated in the microarray analysis in the mammary glands of 2-month-old rats exposed to excess E2 in utero. These include Acta1 (alpha 1 actin), Ccl2 (chemokine (C—C motif) ligand 2), Ckm (muscle creatine kinase), H19/Pro2605 (H19 fetal liver mRNA), Myh13 (heavy polypeptide 13 myosin), Myl2 (light polypeptide 2 myosin) and Pvalb (parvalbumin). Our unpublished results also show several genes were down-regulated, possibly by methylation, in the adult mammary gland of rats whose mothers were fed high birth weight (HBW) inducing high fat diet during pregnancy, including TSGs such as DKK3, DUSP6, HOXD3 and PCAF (FIG. 1). Expression of these genes was increased if the rats were exposed to DNMT inhibitor 5-aza. Importantly, we have found that expression of DNMT1 is elevated in the mammary glands of animals exposed in utero to synthetic estrogen EE2 though a pregnant dam, and the increase persists to at least three subsequent generations.

The events leading to PcGT methylation in women who subsequently develop breast cancer are entirely unknown. We propose this methylation is caused by an exposure to excessively estrogenic environment in utero. In our preliminary study, one third of the down-regulated genes identified by using microarray assays in the mammary glands of adult rats exposed to E2 or high fat diet in utero were PcGTs/TSGs, including H19, RARB, PgR and Rassf1. The process which leads to methylation of PcGTs is initiated by the polycomb repressive unit 2 (PCR2) containing the catalytic subunit polycomb enhancer of zeste-2 (EZH2), and SUZ12 and EED; this complex trimethylates H3K27. Polycombs in PCR1 unit, such as Bmi-1 and Ring1 with YY1 binding protein, recognize chromatin marked with methylated H3K27. Together, PCR1/PCR2/H3K27me3 complex recruits DNA methyltransferases (DNMTs), and consequently methylate PcGTs.

DNMTs are enzymes that methylate CpG islands by adding a methyl group (CH3) onto the 5-carbon of cytosine ring within CpG dinucleotides. These enzymes include DNMT1, DNMT3a, and DNMT3b. DNMT1 functions mainly as a methylation maintenance enzyme, whilst DNMT3a and DNTM3b act as de novo methyltransferases and methylate DNA, but it is now clear that all three can act as maintenance and de novo methylation enzymes. Overexpression of DNMT1 induces genomic hypermethylation and loss of imprinting, and therefore high DNMT1 protein levels may be responsible for aberrant DNA methylation in cancer. RNAi-induced depletion of DNMT1, in turn, leads to demethylation of CpG islands in the breast cancer cells (T47D, MDA-MB-231 and Hs578t).

In support of the view that in utero estrogenic environment may induce sustained DNA methylation, it has been reported that an exposure to endocrine disruptors during fetal period lead to an increase in EZH2 expression in the mammary glands of mice, and SUZ12 in the endometrial tissue. Our preliminary findings indicate that expression of DNMT1 is increased in the mammary glands of rats exposed to a high fat diet or E2 in utero. Therefore, it is possible that both polycombs and DNMTs are affected by increased in utero hormonal environment, and this then causes DNA methylation of PcTGs/TSGs. Emerging evidence suggests that histone and DNA methylation are closely linked, but it is not clear whether histone modifications promote DNA methylation or vice versa. However, since pre-marking PcGTs with H3K27me3 is not sufficient to induce de novo methylation—unmethylated PcGTs in normal cells contain high levels of H3K27me3—it is likely that an increase in DNMT activity and DNA methylation leading to PcGT methylation is caused by other factors than polycombs. However, sustained/increased expression of both polycombs and DNMTs are probably required for PcGTs to remain methylated throughout life or become re-methylated in adult life in normal mammary tissue.

MicroRNAs (miRNAs). DNA methylation can explain persistent silencing of PcGTs and TSGs, but other mechanisms are likely involved in causing persistent up-regulation of oncogenes. The importance of oncogenes in the development of cancer is for example illustrated through genetically modified mouse models that overexpress a specific oncogene and consequently develop mammary tumors. The increase in oncogene expression in human breast tumors is often caused by mutations, but we are not aware of any inherited breast cancers that are linked to a germline mutation in an oncogene. Instead, overexpression of oncogenes in transformed and non-transformed mammary tissue may be caused by down-regulation of miRNAs which target these oncogenes.

miRNAs are short non-coding single stranded RNAs composed of approximately 21-22 nucleotides that regulate gene expression by sequence-specific base-pairing with the 3′-untranslated region (3′UTR) of target mRNAs to induce their post-transcriptional repression, either by inducing inhibition of protein translation or degradation of the mRNA. miRNA target genes can be identified by searching for conserved matches to the seed region of miRNAs. It has been estimated that each of the miRNAs (>800 identified to date) bind to as many as 200 targets, and thus they regulate the expression of at least one third of human mRNAs, and likely more. As a single miRNA can target, and potentially silence, several hundred genes; any given gene can be targeted by several miRNAs. Consequently, a single miRNA is expected to exert only a modest effect on gene expression. However, several single miRNAs have been identified which, when targeted by antisense, are effective in the treatment of, for example high cholesterol (miR-33) or hepatitis C (miR-122).

Similar to DNA methylation, miRNAs are key regulators of normal development, including the development of the mammary gland. However, we are not aware of any studies which have investigated whether any early life exposures alter later miRNA expression. Several miRNAs have been identified which are regulated by estrogens (see below) and which are related to breast cancer. Further, since many miRNAs can be methylated and they also regulate the expression of genes involved in the methylation; i.e., polycombs and DNMTs and oncogenes, it is possible that changes in their expression are involved in mediating the effects of maternal diet during pregnancy on offspring's mammary tumorigenesis.

Estrogens regulate miRNAs. Expression of many miRNAs is affected by estrogens, although the mechanisms involved are not known but may include inhibition of maturation of miRNA from their pri-miRNAs via Drosha and Dicer. Remarkably, miRNAs which in our preliminary study were consistently down-regulated in an adult mammary gland in rats exposed to E2 in utero, compared to control mammary glands, were all same miRNAs which have been reported to be down-regulated by E2 in MCF-7 human breast cancer cells. These findings suggest that high in utero estrogenic environment, by initially down-regulating miRNAs which then leads to up-regulation of genes involved in inducing DNA methylation (polycombs and DNMTs), may explain methylation of PcGTs, TSGs and even miRNAs.

Use of Biomarkers

One aspect of the invention will be to identify individuals at a high risk for cancer due to exposure to an elevated in utero estrogenic environment. Biomarkers could be used to identify these individuals, such as testing for an increase in DNMT1 expression or methylation of normally unmethylated CpG islands of tumor suppressor genes and polycomb target genes in cells obtained from buccal swabs and/or blood. Without being bound by any particular theory, it is believed that excessive levels of estrogens during fetal development might leave an epigenetic imprint in the DNA which causes methylation of normally unmethylated CpG islands. This imprint can be detected not only in the organs where cancer develops, but also in unrelated target tissues, particularly if the stem cells where the epigenetic event occurred during fetal development were present in “cancer” organs and other target tissues.

An additional method to identify individuals at a high risk for cancer due to exposure to an elevated in utero estrogenic environment would be to examine the morphology of specific structures. An exemplary structure is the number of terminal end buds in breast cancer. Both HF and EE2 in utero exposures have been shown to alter mammary gland morphology, mainly by increasing the number of terminal end buds (TEBs). These undifferentiated mammary structures are the sites of malignant transformation in rat mammary gland, and human breast. We examined whether these effects persisted in the F2 and F3 generation offspring (Tables 1 and 2). On postnatal day (PND) 21, the number of TEBs was increased in the mammary glands of both HF and EE2 daughters (F1) (p=0.032 and p=0.033) and granddaughters (F2) (p=0.004 and p=0.006). However, in great-granddaughters (F3), increase in the number of TEBs was only seen in the offspring of EE2 exposed dams (p=0.014). On PND50, the number of TEBs was increased in mammary glands of daughters (F1) (p=0.039) and granddaughters (F2) (p=0. 044), but not great-granddaughters (F3), of HF exposed dams. In the EE2 offspring, however, there were no significance differences in the number of TEBs on PND 50 in any generation.

We also assessed changes in mammary gland morphology in the outcross offspring (Tables 1 and 2). On PND21, mammary glands of Con♀-HF♂ outcross (p=0.005), but not HF♀-Con♂ (p=0.654) had a higher number of TEBs than the controls. Mammary glands of the EE2♀-Con♂ outcross also had a significantly higher number of TEBs (p=0.005) on PND21, but not on PND50 (p=0110). Lower number of TEBs was seen on PND50 in the Con♀-EE2♂ group, compared to the controls (p=0.003).

### REFERENCES

1. Durando, M., Kass, L., Piva, J., Sonnenschein, C., Soto, A. M., Luque, E. H. and Munoz-de-Toro, M. Prenatal bisphenol A exposure induces preneoplastic lesions in the mammary gland in Wistar rats, Environ Health Perspect, 115: 80-86, 2007.
2. Hilakivi-Clarke, L., Cho, E., Onojafe, I., Raygada, M. and Clarke, R. Maternal exposure to genistein during pregnancy increases carcinogen-induced mammary tumorigenesis in female rat offspring, Oncol. Rep., 6: 1089-1095, 1999.
3. Foster, W. G., Younglai, E. V., Boutross-Tadross, O., Hughes, C. L. and Wade, M. G. Mammary gland morphology in Sprague-Dawley rats following treatment with an organochlorine mixture in utero and neonatal genistein, Toxicol. Sci., 77: 91-100, 2004.
4. Hilakivi-Clarke, L., Clarke, R., Onojafe, I., Raygada, M., Cho, E. and Lippman, M. E. A maternal diet high in n-6 polyunsaturated fats alters mammary gland development, puberty onset, and breast cancer risk among female rat offspring, Proc. Natl Acad. Sci. USA, 94: 9372-9377, 1997.
5. Boylan, E. S. and Calhoon, R. E. Mammary tumorigenesis in the rat following prenatal exposure to diethylstilbestrol and postnatal treatment with 7,12-dimethylbenz[a]anthracene, J. Toxicol. Environ. Health, 5:1059-1071, 1979.
6. Boylan, E. S. and Calhoon, R. E. Prenatal exposure to diethylstilbestrol: ovarian-independent growth of mammary tumors induced by 7,12-dimethylbenz[a]anthracene, J. Natl. Cancer Inst., 66: 649-652, 1981.
7. Boylan, E. S. and Calhoon, R. E. Transplacental action of diethylstilbestrol on mammary carcinogenesis in female rats given one or two doses of 7,12-dimethylbenz(a)anthracene, Cancer Res., 43: 4879-4884, 1983.
8. Rothschild, T. C., Boylan, E. S., Calhoon, R. E. and Vonderhaar, B. K. Transplacental effects of diethylstilbestrol on mammary development and tumorigenesis in female ACI rats, Cancer Res., 47: 4508-4516, 1987.
9. Walker, B. E. No effect of adult dietary fat on tumors induced prenatally by diethylstilbestrol, Nutr. Cancer, 17: 161-169, 1992.
10. Vassilacopoulou, D. and Boylan, E. S. Mammary gland morphology and responsiveness to regulatory molecules following prenatal exposure to diethylstilbestrol, Teratog. Carcnog. Mutagen., 13: 59-74, 1993.
11. Kawaguchi, H., Miyoshi, N., Miyamoto, Y., Souda, M., Umekita, Y., Yasuda, N. and Yoshida, H. Effects of fetal exposure to diethylstilbestrol on mammary tumorigenesis in rats, J. VetMed. Sci., 71: 1599-1608, 2009.
12. Ekbom, A., Trichopoulos, D., Adami, H. O., Hsieh, C. C. and Lan, S. J. Evidence of prenatal influences on breast cancer risk, Lancet, 340: 1015-1018, 1992.
13. Sanderson, M., Williams, M., Malone, K. E., Stanford, J. L., Emanuel, I., White, E. and Daling, J. R. Perinatal factors and risk of breast cancer, Edidemiology, 7: 34-37, 1996.
14. Weiss, H. A., Potischman, N., Brinton, L. A., Brogan, D., Coates, R. J., Gammon, M. D., Malone, K. E. and Schoenberg, J. B. Prenatal and perinatal risk factors for breast cancer in young women, Edidemiology, 8: 181-187, 1997.
15. Palmer, J. R., Wise, L. A., Hatch, E. E., Troisi, R., Titus-Emstoff, L., Strohsnitter, W., Kaufman, R., Herbst, A L, Noller, K L., Hyer, M. and Hoover, R. N. Prenatal diethylstilbestrol exposure and risk of breast cancer, Cancer Epidemiol. Biomarkers Prev., 15: 1509-1514, 2006.
16. amanti-Kandarakis, E., Bourguignon, J. P., Giudice, L. C., Hauser, R., Prins, G. S., Soto, A M., Zoeller, R. T. and Gore, A C. Endocrine-disrupting chemicals: an Endocrine Society scientific statement, Endocr. Rev., 30: 293-342, 2009.
17. Prins, G. S. Estrogen imprinting: when your epigenetic memories come back to haunt you, Endocrinology, 149: 5919-5921, 2008.
18. Tang, W. Y., Newbold, R., Mardilovich, K., Jefferson, W., Cheng, R. Y., Medvedovic, M. and Ho, S. M. Persistent hypomethylation in the promoter of nucleosomal binding protein 1 (Nsbp1) correlates with overexpression of Nsbp1 in mouse uteri neonatally exposed to diethylstilbestrol or genistein, Endocrinology, 2008.
19. Ho, S. M., Tang, W. Y., Belmonte, d. F. and Prins, G. S. Developmental exposure to estradiol and bisphenol A increases susceptibility to prostate carcinogenesis and epigenetically regulates phosphodiesterase type 4 variant 4, Cancer Res, 66: 5624-5632, 2006.
20. Fourkala, E. O., Hauser-Kronberger, C., Apostolidou, S., Bumell, M., Jones, A, Grall, J., Reitsamer, R., Fiegl, H., Jacobs, I., Menon, U. and Widschwendter, M. DNA methylation of polycomb group target genes in cores taken from breast cancer centre and periphery, Breast Cancer Res Treat, 120: 345-355, 2010.
21. Teschendorff, A. E., Menon, U., Gentry-Maharaj, A., Ramus, S. J., Weisenberger, D. J., Shen, H., Campan, M., Noushmehr, H., Bell, C. G., Maxwell, A. P., Savage, D. A., Mueller-Holzner, E., Marth, C., Kocjan, G., Gayther, S. A., Jones, A, Beck, S., Wagner, W., Laird, P. W., Jacobs, I. J. and Widschwendter, M. Age-dependent DNA methylation of genes that are suppressed in stem cells is a hallmark of cancer, Genome Res, 2010.
22. Vasilatos, S. N., Broadwater, G., Barry, W. T., Baker, J. C., Jr., Lem, S., Dietze, E. C., Bean, G. R., Bryson, A D., Pilie, P. G., Goldenberg, V., Skaar, D., Paisie, C., Torres-Hernandez, A, Grant, T. L., Wilke, L. G., Ibarra-Drendall, C., Ostrander, J. H., D'Amato, N. C., Zalles, C., Jirtle, R., Weaver, V. M. and Seewaldt, V. L. CpG island tumor suppressor promoter methylation in non-BRCA-associated early mammary carcinogenesis, Cancer Epidemiol. Biomarkers Prev., 18: 901-914, 2009.
23. Yin, Y., Bai, R., Russell, R. G., Beildeck, M. E., Xie, Z., Kopelovich, L. and Glazer, R. I. Characterization of medroxyprogesterone and DMBA-induced multilineage mammary tumors by gene expression profiling, Mol. Carcinog., 44: 42-50, 2005.
24. Chavez-Blanco, A, Perez-Plasencia, C., Perez-Cardenas, E., Carrasco-Legleu, C., Rangel-Lopez, E., Segura-Pacheco, B., Taja-Chayeb, L., Trejo-Becerril, C., Gonzalez-Fierro, A, Candelaria, M., Cabrera, G. and Duenas-Gonzalez, A. Antineoplastic effects of the DNA methylation inhibitor hydralazine and the histone deacetylase inhibitor valproic acid in cancer cell lines, Cancer Cell Int, 6: 2, 2006.
25. Hubaux, R., Vandermeers, F., Crisanti, M. C., Kapoor, V., Burny, A., Mascaux, C., Albelda, S. M. and Willems, L. Preclinical evidence for a beneficial impact of valproate on the response of small cell lung cancer to first-line chemotherapy, Eur. J Cancer, 46: 1724-1734, 2010.
26. Mannaerts, I., Nuytten, N. R., Rogiers, V., Vanderkerken, K., van Grunsven, L. A. and Geerts, A. Chronic administration of valproic acid inhibits activation of mouse hepatic stellate cells in vitro and in vivo, Hepatology, 51: 603-614, 2010.
27. Wang, X. Y., Yin, Y., Yuan, H., Sakamaki, T., Okano, H. and Glazer, R. I. Musashil modulates mammary progenitor cell expansion through proliferin-mediated activation of the Wnt and Notch pathways, Mol. Cell Biol., 2008.
28. Anway M D, Skinner M K. Epigenetic transgenerational actions of endocrine disruptors. Endocrinology 2006 June; 147(6 Suppl):S43-S49.
29. Tang W Y, Newbold R, Mardilovich K, Jefferson W, Cheng R Y, Medvedovic M, et al. Persistent hypomethylation in the promoter of nucleosomal binding protein 1 (Nsbp1) correlates with overexpression of Nsbp1 in mouse uteri neonatally exposed to diethylstilbestrol or genistein. Endocrinology 2008 Jul. 31.
30. Marselos M, Tomatis L. Diethylstilbestrol: II, Pharmacology, toxicology and carcinogenity in experimental animals. Eur J Cancer 1993; 29A:149-55.
31. Jenkins S, Raghuraman N, Eitoum I, Carpenter M, Russo J, Lamartiniere C A. Oral exposure to bisphenol a increases dimethylbenzanthracene-induced mammary cancer in rats. Environ Health Perspect 2009 June; 117(6):910-5.
32. Hilakivi-Clarke L. Nutritional modulation of terminal end buds: its relevance to breast cancer prevention. Curr Cancer Drug Targets 2007 August; 7(5):465-74.
33. Arce C, Perez-Plasencia C, Gonzalez-Fierro A, de IC-H, Revilla-Vazquez A, Chavez-Blanco A, et al. A proof-of-principle study of epigenetic therapy added to neoadjuvant doxorubicin cyclophosphamide for locally advanced breast cancer. PLoS ONE 2006; 1:e98.
34. Matsushima A, Kakuta Y, Teramoto T, Koshiba T, Liu X, Okada H, et al. Structural evidence for endocrine disruptor bisphenol A binding to human nuclear receptor ERR gamma J Biochem 2007 October 142(4):517-24.
35. Palmer J R, Wise L A, Hatch E E, Troisi R, Titus-Emstoff L, Strohsnitter W, et al. Prenatal diethylstilbestrol exposure and risk of breast cancer. Cancer Epidemiol Biomarkers Prev 2006 August; 15(8): 1509-14.
36. Durando M, Kass L, Piva J, Sonnenschein C, Soto A M, Luque E H, et al. Prenatal bisphenol A exposure induces preneoplastic lesions in the mammary gland in Wistar rats. Environ Health Perspect 2007 January; 115(1):80-6.
37. Russo J, Hasan L M, Balogh G, Guo S, Russo I H. Estrogen and its metabolites are carcinogenic agents in human breast epithelial cells. J Steroid Biochem Mol Biol 2003 October; 87(1):1-25.
38. Gould J C, Leonard L S, Maness S C, Wagner B L, Conner K, Zacharewski T, et al. Bisphenol A interacts with the estrogen receptor alpha in a distinct manner from estradiol. Mol Cell Endocrinol 1998 Jul. 25; 142(1-2):203-14.
39. Yamashita S. Expression of estrogen-regulated genes during development in the mouse uterus exposed to diethylstilbestrol neonatally. Curr Pharm Des 2006; 12(12):1505-20.
40. Gowda K, Marks B D, Zielinski T K, Ozers M S. Development of a coactivator displacement assay for the orphan receptor estrogen-related receptor-gamma using time-resolved fluorescence resonance energy transfer. Anal Biochem 2006 Oct. 1; 357(1):105-15.
41. Suetsugi M, Su L, Kartsberg K, Yuan Y C, Chen S. Flavone and isoflavone phytoestrogens are agonists of estrogen-related receptors. Mol Cancer Res 2003 November; 1(13):981-91.
42. Takashima-Sasaki K, Mon C, Komiyama M. Exposure of juvenile female mice to isoflavone causes lowered expression of estrogen-related receptor gamma gene in vagina. Reprod Toxicol 2007 June; 23(4):507-12.
43. Kuiper G G, Lemmen J G, Carisson B, Corton J C, Safe SH, van der Saag P T, et al. Interaction of estrogenic chemicals and phytoestrogens with estrogen receptor beta. Endocrinology 1998 October 139(10):4252-63.
44. Ariazi E A, Clark G M, Mertz J E. Estrogen-related receptor alpha and estrogen-related receptor gamma associate with unfavorable and favorable biomarkers, respectively, in human breast cancer. Cancer Res 2002 Nov. 15; 62(22):6510-8.
45. Huss J M, Kopp R P, Kelly D P. Peroxisome proliferator-activated receptor coactivator-1alpha (PGC-1alpha) coactivates the cardiac-enriched nuclear receptors estrogen-related receptor-alpha and -gamma. Identification of novel leucine-rich interaction motif within PGC-1alpha. J Biol Chem 2002 Oct. 25; 277(43):40265-74.
46. Liu D, Zhang Z, Teng C T. Estrogen-related receptor-gamma and peroxisome proliferator-activated receptor-gamma coactivator-1alpha regulate estrogen-related receptor-alpha gene expression via a conserved multi-hormone response element J Mol Endocrinol 2005 April; 34(2):473-87.
47. Zhang Y, Ma K, Sadana P, Chowdhury F, Gaillard S, Wang F, et al. Estrogen-related receptors stimulate pyruvate dehydrogenase kinase isoform 4 gene expression. J Biol Chem 2006 Dec. 29; 281(52): 39897-906.
48. Soriano F X, Liesa M, Bach D, Chan D C, Palacin M, Zorzano A. Evidence for a mitochondrial regulatory pathway defined by peroxisome proliferator-activated receptor-gamma coactivator-1 alpha, estrogen-related receptor-alpha, and mitofusin 2. Diabetes 2006 June; 55(6):1783-91.
49. Riggins R B, Lan J P, Klimach U, Zwart A, Cavalli L R, Haddad B R, et al. ERRgamma mediates tamoxifen resistance in novel models of invasive lobular breast cancer. Cancer Res 2008 November 1; 68(21):8908-17.
50. Edwards T M, Myers J P. Environmental exposures and gene regulation in disease etiology. Environ Health Perspect 2007 September; 115(9):1264-70.
51. Santos F, Dean W. Epigenetic reprogramming during early development in mammals. Reproduction 2004 June; 127(6):643-51.
52. Block K, Kardana A, Igarashi P, Taylor HS. In utero diethylstilbestrol (DES) exposure alters Hox gene expression in the developing mullerian system. FASEB J 2000 June; 14(9):1101-8.
53. Li S, Ma L, Chiang T, Burow M, Newbold R R, Negishi M, et al. Promoter CpG methylation of Hox-a10 and Hox-a11 in mouse uterus not altered upon neonatal diethylstilbestrol exposure. Mol Carcinog 2001 December; 32(4):213-9.
54. Li S, Hansman R, Newbold R, Davis B, McLachlan J A, Barrett J C. Neonatal diethylstilbestrol exposure induces persistent elevation of c-fos expression and hypomethylation in its exon-4 in mouse uterus. Mol Carcinog 2003 October; 38(2):78-84.
55. Cooney C A, Dave A A, Wolff GL. Maternal methyl supplements in mice affect epigenetic variation and DNA methylation of offspring. J Nutr 2002 August; 132(8 Suppl):2393S-400S.
56. Dolinoy D C, Weidman J R, Waterland R A, Jirtle R L. Maternal genistein alters coat color and protects avy mouse offspring from obesity by modifying the fetal epigenome. Environ Health Perspect 2006 April, 114(4):567-72.
57. Plachot C, Lelievre SA. DNA methylation control of tissue polarity and cellular differentiation in the mammary epithelium. Exp Cell Res 2004 Aug. 1; 298(1):122-32.
58. Ho S M, Tang W Y, Belmonte d F, Prins GS. Developmental exposure to estradiol and bisphenol A increases susceptibility to prostate carcinogenesis and epigenetically regulates phosphodiesterase type 4 variant 4. Cancer Res 2006 Jun. 1; 66(11):5624-32.
59. Silva I S, De Stavola B, McCormack V. Birth size and breast cancer risk: re-analysis of individual participant data from 32 studies. PLoS Med 2008 Sep. 30; 5(9):e193.
60. Russo J, Russo I H. Biological and molecular bases of mammary carcinogenesis. Lab Invest 1987; 57:112-37.
61. Russo J, Hu Y F, Yang X, Russo I H. Developmental, cellular, and molecular basis of human breast cancer. J Nati Cancer Inst Monogr 2000; (27):17-37.
62. Russo J, Russo IH. Influence of differentiation and cell kinetics on the susceptibility of the rat mammary gland to carcinogenesis. Cancer Res 1980 August; 40(8 Pt 1):2677-87.
63. Telang N T, Suto A, Wong G Y, Osborne M P, Bradlow H L. Induction by estrogen metabolite 16 alpha-hydroxyestrone of genotoxic damage and aberrant proliferation in mouse mammary epithelial cells. J Nati Cancer Inst 1992 Apr. 15; 84(8):634-8.
64. Hilakivi-Clarke L, Onojafe I, Raygada M, Cho E, Clarke R, Lippman M. Breast cancer risk in rats fed a diet high in n-6 polyunsaturated fatty acids during pregnancy. J Natl Cancer Inst 1996; 88:1821-7.
65. de Assis S, Galam K, Hilakivi-Clarke L. High birth weight increases mammary tumorigenesis in rats. Int J Cancer 2006; 119:1537-46.
66. Paguirigan A, Beebe D J, Liu B, Alexander C. Mammary stem and progenitor cells: tumour precursors? Eur J Cancer 2006 June; 42(9):1225-36.
67. Parsa S, Ramasamy S K, De Langhe S, Gupte W, Haigh J J, Medina D, et al. Terminal end bud maintenance in mammary gland is dependent upon FGFR2b signaling. Dev Biol 2008 May 1; 317(1):121-31.
68. Hilakivi-Clarke L, Cho E, Raygada M, Kenney N. Alterations in mammary gland development following neonatal exposure to estradiol, transforming growth factor alpha, and estrogen receptor antagonist ICI 182, 780. J Cell Physiol 1997; 170:279-89.
69. Bird A. DNA methylation patterns and epigenetic memory. Genes Dev 2002 Jan. 1; 16(1):6-21.
70. Jones P A, Baylin S B. The epigenomics of cancer. Cell 2007 Feb. 23; 128(4):683-92.
71. Gargiulo G, Minucci S. Epigenomic profiling of cancer cells. Int J Biochem Cell Biol 2009 January; 41(1):127-35.
72. Weber M, Davies J J, Wittig D, Oakeley E J, Haase M, Lam W L, et al. Chromosome-wide and promoter-specific analyses identify sites of differential DNA methylation in normal and transformed human cells. Nat Genet 2005 August; 37(8):853-62.
73. Zilberman D, Gehring M, Tran R K, Ballinger T, Henikoff S. Genome-wide analysis of Arabidopsis thaliana DNA methylation uncovers an interdependence between methylation and transcription. Nat Genet. 2007 January; 39(1):61-9.
74. Tennis M, Krishnan S, Bonner M, Ambrosone C B, Vena J E, Moysich K, et al. p53 Mutation analysis in breast tumors by a DNA microarray method. Cancer Epidemiol Biomarkers Prev 2006 January; 15(1):80-5.
75. Tao M H, Shields P G, Nie J, Millen A, Ambrosone C B, Edge S B, et al. DNA hypermethylation and clinicopathological features in breast cancer the Western New York Exposures and Breast Cancer (WEB) Study. Breast Cancer Res Treat 2009 April; 114(3):559-68.
76. Tost J, Gut IG. DNA methylation analysis by pyrosequencing. Nat Protoc 2007; 2(9):2265-75.
77. Wang Y, Miller D J, Clarke R. Approaches to working in high-dimensional data spaces: gene expression microarrays. Br J Cancer 2008 Mar. 25; 98(6):1023-8.
78. Liu J J, Cutler G, Li W, Pan Z, Peng S, Hoey T, et al. Multiclass cancer classification and biomarker discovery using GA-based algorithms. Bioinformatics 2005 Jun. 1; 21(11):2691-7.
79. Xuan J, Wang Y, Dong Y, Feng Y, Wang B, Khan J, et al. Gene selection for multiclass prediction by weighted fisher criterion. EURASIP J Bioinform Syst Biol 2007; 64628.
80. Lai C, Reinders M J, van't Veer U, Wessels L F. A comparison of univariate and multivariate gene selection techniques for classification of cancer datasets. BMC Bioinformatics 2006; 7:235.
81. Shedden K A, Taylor J M, Giordano T J, Kuick R, Misek D E, Rennert G, et al. Accurate molecular classification of human cancers based on gene expression using a simple classifier with a pathological tree-based framework. Am J Pathol 2003 November; 163(5):1985-95.
82. Wang J, Li H, Zhu Y, Yousef M, Nebozhyn M, Showe M, et al. VISDA: an open-source caBIG analytical tool for data clustering and beyond. Bioinformatics 2007 Aug. 1; 23(15):2024-7.
83. Clarke R, Ressom H W, Wang A, Xuan J, Liu M C, Gehan E A, et al. The properties of high-dimensional data spaces: implications for exploring gene and protein expression data. Nat Rev Cancer 2008 January; 8(1):37-49.
84. Chavez-Blanco A, Perez-Plasencia C, Perez-Cardenas E, Carrasco-Legleu C, Rangel-Lopez E, Segura-Pacheco B, et al. Antineoplastic effects of the DNA methylation inhibitor hydralazine and the histone deacetylase inhibitor valproic acid in cancer cell lines. Cancer Cell Int 2006; 6:2.
85. Rajkumar L, Kittrell F S, Guzman R C, Brown P H, Nandi S, Medina D. Hormone-induced protection of mammary tumorigenesis in genetically engineered mouse models. Breast Cancer Res 2007; 9(1):R12.
86. Russo I H, Russo J. Primary prevention of breast cancer by hormone-induced differentiation. Recent Results Cancer Res 2007; 174:111-30.
87. Kinnunen T I, Pasanen M, Aittasalo M, Fogelholm M, Hilakivi-Clarke L, Weiderpass E, et al. Preventing excessive weight gain during pregnancy—a controlled trial in primary health care. Eur J Clin Nutr 2007 July; 61(7):884-91.
88. Cui Y, Lu C, Liu L, Sun D, Yao N, Tan S, et al. Reactivation of methylation-silenced tumor suppressor gene p16INK4a by nordihydroguaiaretic acid and its implication in G1 cell cycle arrest. Life Sci 2008 Jan. 30; 82(5-6):247-55.
89. Salehi, F., Tumer, M. C., Phillips, K. P., Wigle, D. T., Krewski, D. and Aronson, K. J. Review of the etiology of breast cancer with special attention to organochlorines as potential endocrine disruptors, J Toxicol Environ Health B Crit. Rev., 11: 276-300, 2008.
90. Durando, M., Kass, L., Piva, J., Sonnenschein, C., Soto, A M., Luque, E. H. and Munoz-de-Toro, M. Prenatal bisphenol A exposure induces preneoplastic lesions in the mammary gland in Wistar rats, Environ Health Perspect, 115: 80-86, 2007.
91. Prins, G. S. Estrogen imprinting: when your epigenetic memories come back to haunt you, Endocrinology, 149: 5919-5921, 2008.
92. de, A S., Ward, A, Cruz, M. I. and Hilakivi-Clarke, L Changes in mammary gland morphology and breast cancer risk in rats, J. Vis. Exp., 2010.
93. Angelova V, Ivanova R, Delibaltova V and Ivanov K Bio-accumulation and distribution of heavy metals in fibre crops (flax, cotton, hemp), Industrial Crops and Products, 19: 197-205, 2004.
94. Jefferson, W. N., Doerge, D., Padilla-Banks, E., Woodling, K. A., Kissling, G. E. and Newbold, R. Oral exposure to genistin, the glycosylated form of genistein, during neonatal life adversely affects the female reproductive system, Environ. Health Perspect., 117: 1883-1889, 2009.
95. Levy, J. R., FAber, K. A., Ayyash, L. and Hughers, C. L. Jr. The effect of prenatal exposure to the phytoesfrogen genistein on sexual differentiation in rats, Proc Soc Exp Biol Med, 208: 60-66, 1995.
96. Munoz-de-Toro, M., Markey, C. M., Wadia, P. R., Luque, E. H., Rubin, B. S., Sonnenschein, C. and Soto, A. M. Perinatal exposure to bisphenol-A alters peripubertal mammary gland development in mice, Endocrinology, 146: 4138-4147, 2005.
97 Larson, P. S., Schlechter, B. L., de las, M. A., Garber, J. E., Cupples, L. A. and Rosenberg, C. L. Allele imbalance, or loss of heterozygosity, in normal breast epithelium of sporadic breast cancer cases and BRCA1 gene mutation carriers is increased compared with reduction mammoplasty tissues, J. Clin. Oncol., 23: 8613-8619, 2005.
98. Kristensen, L. S., Nielsen, H. M. and Hansen, L. L. Epigenetics and cancer treatment, Eur. J. Pharmacol., 625: 131-142, 2009.
99. Widschwendter, M., Apostolidou, S., Raum, E., Rothenbacher, D., Fiegl, H., Menon, U., Stegmaier, C., Jacobs, I. J. and Brenner, H. Epigenotyping in peripheral blood cell DNA and breast cancer risk: a proof of principle study, PLoS. ONE., 3: e2656, 2008.
100. Jirtle, R. L. & Skinner, M. K. Environmental epigenomics and disease susceptibility. Nat. Rev. Genet. 8, 253-262 (2007).
101. Familial breast cancer collaborative reanalysis of individual data from 52 epidemiological studies including 58, 209 women with breast cancer and 101,986 women without the disease. Lancet 358, 1389-1399 (2001).
102. Antoniou, A. C. & Easton, D. F. Models of genetic susceptibility to breast cancer. Oncogene 25, 5898-5905 (2006).
103. Oldenburg, R. A., Meijers-Heijboer, H., Cornelisse, C. J. & Devilee, P. Genetic susceptibility for breast cancer how many more genes to be found? Crit. Rev. Oncol. Hematol. 63, 125-149 (2007).
104. Kyle, U. G. & Pichard, C. The Dutch Famine of 1944-1945: a pathophysiological model of long-term consequences of wasting disease. Curr. Opin. Clin. Nutr. Metab Care 9, 388-394 (2006).
105. Walker, B. E. & Kurth, L. A. Increased reproductive tract tumors in the female offspring of mice fed a high fat diet during the fetal stage of pregnancy. Cancer Lett. 97, 57-60 (1995).
106. Rothschild, T. C., Boylan, E. S., Calhoon, R. E. & Vonderhaar, B. K. Transplacental effects of diethylstilbestrol on mammary development and tumorigenesis in female ACI rats. Cancer Res. 47, 4508-4516 (1987).
107. Lopez, J., Ogren, L., Verjan, R. & Talamantes, F. Effects of perinatal exposure to a synthetic estrogen and progestin on mammary tumorigenesis in mice. Teratology 38, 129-134 (1988).
108. Titus-Emstoff, L. et al. Long-term cancer risk in women given diethylstilbestrol (DES) during pregnancy. Br. J. Cancer 84, 126-133 (2001).
109. Palmer, J. R. et al. Risk of breast cancer in women exposed to diethylstilbestrol in utero: prelimiinary results (United States). Cancer Causes Control 13, 753-758 (2002).
110. Anway, M. D., Cupp, A. S., Uzumcu, M. & Skinner, M. K. Epigenetic transgenerational actions of endocrine disruptors and male fertility. Science 308, 1466-1469 (2005).
111. Anway, M. D. & Skinner, M. K. Epigenetic transgenerational actions of endocrine disruptors. Endocrinology 147, S43-S49 (2006).
112. Anway, M. D., Memon, M. A., Uzumcu, M. & Skinner, M. K. Transgenerational effect of the endocrine disruptor vinclozolin on male spermatogenesis. J. Androl 27, 868-879 (2006).
113. Russo, I. H. & Russo, J. Mammary gland neoplasia in long-term rodent studies. Environ. Health Perspect. 104, 938-967 (1996).
114. Skinner, M. K. Role of epigenetics in developmental biology and transgenerational inheritance. Birth Defects Res. C. Embryo. Today 93, 51-55 (2011).
115. Hsu, D. W. et al. Two major forms of DNA (cytosine-5) methyltransferase in human somatic tissues. Proc. Natl. Acad. Sci. U.S.A 96, 9751-9756 (1999).
116. Jurkowska, R. Z., Jurkowski, T. P. & Jeltsch, A. Structure and function of mammalian DNA methyltransferases. Chembiochem. 12, 206-222 (2011).
117. Feltus, F. A., Lee, E. K., Costello, J. F., Plass, C. & Vertino, P. M. Predicting aberrant CpG island methylation. Proc. Natl. Acad. Sci. U.S.A 100, 12253-12258 (2003).
118. Jair, K. W. et al. De novo CpG island methylation in human cancer cells. Cancer Res. 66, 682-692 (2006).
119. Lillycrop, K. A. et al. Induction of altered epigenetic regulation of the hepatic glucocorticoid receptor in the offspring of rats fed a protein-restricted diet during pregnancy suggests that reduced DNA methyltransferase-1 expression is involved in impaired DNA methylation and changes in histone modifications. Br. J. Nutr. 97, 1064-1073 (2007).
120. Aflatoonian, B. & Moore, H. Germ cells from mouse and human embryonic stem cells. Reproduction. 132, 699-707 (2006).
121. Schulz, R. et al. The parental non-equivalence of imprinting control regions during mammalian development and evolution. PLoS. Genet. 6, ei001214 (2010).
122. Borgel, J. et al. Targets and dynamics of promoter DNA methylation during early mouse development Nat. Genet. 42, 1093-1100 (2010).
123. Olivo, S. E. & Hilakivi-Clarke, L. Opposing effects of prepubertal low- and high-fat n-3 polyunsaturated fatty acid diets on rat mammary tumorigenesis. Carcinogenesis 26, 1563-1572 (2005).
124. Ghoshal, K. et al. A folate- and methyl-deficient diet alters the expression of DNA methyltransferases and methyl CpG binding proteins involved in epigenetic gene silencing in livers of F344 rats. J. Nutr. 136, 1522-1527 (2006).
125. Hoover R N, Hyer M, Pfeiffer R M, Adam E, Adam E, Bond B, Cheville A L, Colton T, Hartge P, Hatch E E, Herbst A L, Karlan B Y, Kaufman R, Noller K L, Palmer J R, Robboy S J, Saal R C, Strohsnitter W, Titus-Emstoff L, Troisi R Adverse health outcomes in women exposed in utero to diethylstilbestrol. N Engl J. Med. 2011 Oct. 6; 365(14):1304-14
126. Sato, K., Fukata, H., Kogo, Y., Ohgane, J., Shiota, K. and Mori, C. Neonatal exposure to diethylstilbestrol alters expression of DNA methyltransferases and methylation of genomic DNA in the mouse uterus, Endocr. J, 56: 131-139, 2009.
127. A. Y. So, J. W. Jung, S. Lee, H. S. Kim, and K. S. Kang. DNA methyltransferase controls stem cell aging by regulating BMI1 and EZH2 through microRNAs. PLoS One 6 (5):e19503, 2011.
128. Sato, K., Fukata, H., Kogo, Y., Ohgane, J., Shiota, K. and Mori, C. Neonatal exposure to diethylstilbestrol alters the expression of DNA methyltransferases and methylation of genomic DNA in the epididymis of mice, Endocr. J, 53: 331-337, 2006.
129. Sandhu, A. G. Rivenbark, and W. B. Coleman. Enhancement of chemotherapeutic efficacy in hypermethylator breast cancer cells through targeted and pharmacologic inhibition of DNMT3b. Breast Cancer Res Treat., 2011.
130. Cheng, A. S. et al. Epithelial progeny of estrogen-exposed breast progenitor cells display a cancer-like methylome. Cancer Res. 68, 1786-1796 (2008).
131. Moelans, C. B., Verschuur-Maes, A. H. & van Diest, P. J. Frequent promoter hypermethylation of BRCA2, CDH13, MSH6, PAX5, PAX6 and WT1 in ductal carcinoma in situ and invasive breast cancer. J. Pathol. 225, 222-231 (2011).
132. Jiang, Y., Tong, D., Lou, G., Zhang, Y. & Geng, J. Expression of RUNX3 gene, methylation status and clinicopathological significance in breast cancer and breast cancer cell lines. Pathobiology 75, 244-251 (2008).
133. Park, S. Y. at al. Promoter CpG island hypermethylation during breast cancer progression. Virchows Arch. 458, 73-84 (2011).
134. Teschendorff, A. E. at al. Age-dependent DNA methylation of genes that are suppressed in stem cells is a hallmark of cancer. Genome Res. 20, 440-446 (2010).
135. Cheng, A. S. et al. Epithelial progeny of estrogen-exposed breast progenitor cells display a cancer-like methylome. Cancer Res. 68, 1786-1796 (2008).

What is claimed:

1. A method of reducing breast cancer risk in an individual exposed in utero to an elevated estrogenic environment, the method comprising identifying said individual by obtaining a measurement of at least one of: (a) DNMT1 expression, (b) methylation of normally unmethylated CpG islands of tumor suppressor genes and (c) polycomb target genes, in cells obtained from the individual and comparing the measurement to a statistical level of at least one of (a), (b) and (c) in a population of individuals not exposed in utero to such an elevated estrogenic environment.

2. The method of claim 1, wherein the statistical level is a percentage of the mean value of a population not exposed in utero to an elevated estrogenic environment.

3. The method of claim 2, wherein the percentage is selected from the group consisting of about 150%, about 175% and about 200%.

4. The method of claim 1, wherein the statistical level is a confidence interval around the mean of a population not exposed in utero to an elevated estrogenic environment.

5. The method of claim 4, wherein the confidence interval is selected from the group consisting of about 50%, about 60%, about 70%, about 75%, about 80%, about 85%, about 90% and about 95%.

6. A method of reducing breast cancer risk in an individual exposed in utero to an elevated estrogenic environment by administering at least one DNA methyltransferase (DNMT) and/or histone deacetylase (HDAC) inhibitor to an individual identified by obtaining a measurement of at least one of (a) DNMT1 expression; (b) methylation of normally unmethylated CpG islands of tumor suppressor genes; and (c) polycomb target genes, in cells obtained from the individual and comparing the measurement to a statistical level of at least one of (a), (b) and (c) in a population of individuals not exposed in utero to such an elevated estrogenic environment.

7. The method of claim 6, wherein the at least one DNA methyltransferase (DNMT) inhibitor is selected from the group consisting of 5-azacytidine, 5-aza-2′-deoxycytidine, fazarabine, DHAC, Ara-C, zebularine, (−)-epigallocatechin-3-gallate, MG98, RG108, and procainamid combinations.

8. The method of claim 6, wherein the at least one histone deacetylase (HDAC) inhibitor is selected from the group consisting of small molecular weight carboxylates; hydroxamic acids; benzamides; and cyclic peptides.

9. The method of claim 6, wherein the statistical level is a percentage of the mean value of a population not exposed in utero to an elevated estrogenic environment.

10. The method of claim 9, wherein the percentage is selected from the group consisting of about 150%, about 175% and about 200%.

11. The method of claim 6, wherein the statistical level is a confidence interval around the mean of a population not exposed in utero to an elevated estrogenic environment.

12. The method of claim 11, wherein the confidence interval is selected from the group consisting of about 50%, about 60%, about 70%, about 75%, about 80%, about 85%, about 90% and about 95%.

13. A method of reducing breast cancer risk in an individual exposed in utero to an elevated estrogenic environment by administering at least one DNA methyltransferase (DNMT) and/or histone deacetylase (HDAC) inhibitor to an individual identified by obtaining a measurement of at least one of: (a) DNMT1 expression; (b) methylation of normally unmethylated CpG islands of tumor suppressor genes; and (c) polycomb target genes, in cells obtained from the individual, comparing the measurement to a statistical level of at least one of (a), (b) or (c) in a population of individuals not exposed in utero to such an elevated estrogenic environment, wherein the inhibitor administered is selected based on the efficacy obtained by treating cells from the individual with several DNA methyltransferase (DNMT) and/or histone deacetylase (HDAC) inhibitors and selecting the best inhibitor for the individual.

14. The method of claim 13, wherein the at least one DNA methyltransferase (DNMT) is selected from the group consisting of 5-azacytidine, 5-aza-2′-deoxycytidine, fazarabine, DHAC, Ara-C, zebularine, (−)-epigallocatechin-3-gallate, MG98, RG108, and procainamid combinations

15. The method of claim 13, wherein the at least one histone deacetylase (HDAC) inhibitor is selected from the consisting of small molecular weight carboxylates; hydroxamic acids; benzamides; and cyclic peptides

16. The method of claim 13, wherein the statistical level is a percentage of the mean value of a population not exposed in utero to an elevated estrogenic environment.

17. The method of claim 16, wherein the percentage is selected from the group consisting of 150%, 175% and 200%.

18. The method of claim 13, wherein the statistical level is a confidence interval around the mean of a population not exposed in utero to an elevated estrogenic environment.

19. The method of claim 18, wherein the confidence interval is selected from the group consisting of 50%, 60%, 70%, 75%, 80%, 85%, 90% and 95%.

20. A method of reducing breast cancer risk in an individual exposed in utero to an elevated estrogenic environment by administering at least one DNA methyltransferase (DNMT) and/or histone deacetylase (HDAC) inhibitor to an individual as part of a treatment regime which may include the administration of at least one anti-cancer agent.

21. The method of claim 6, wherein the at least one DNA methyltransferase (DNMT) and/or histone deacetylase (HDAC) inhibitor is selected from the group consisting of valproic acid, vorinostat, and hydralazine.
